SEQUENCE LISTING (36 sequences)

SEQ ID NO: 1 - protein, 7 aa, Artificial Sequence
  CDR_L2 of an anti-Ryk antibody or antibody derivative
RANRLVE

SEQ ID NO: 2 - protein, 8 aa, Artificial Sequence
  CDR_H2 of an anti-Ryk antibody or antibody derivative
STGGGGTY

SEQ ID NO: 3 - protein, 7 aa, Artificial Sequence
  CDR_H3_1 of an anti-Ryk antibody or antibody derivative
HGDSGDY

SEQ ID NO: 4 - protein, 7 aa, Artificial Sequence
  CDR_H3_2 of an anti-Ryk antibody or antibody derivative
HGDQGDY

SEQ ID NO: 5 - protein, 11 aa, Artificial Sequence
  CDR_L1 of an anti-Ryk antibody or antibody derivative
KASQDINSYLS

SEQ ID NO: 6 - protein, 9 aa, Artificial Sequence
  CDR_L3 of an anti-Ryk antibody or antibody derivative
LQYDEFPLT

SEQ ID NO: 7 - protein, 7 aa, Artificial Sequence
  CDR_H1 of an anti-Ryk antibody or antibody derivative
GFTFSSY

SEQ ID NO: 8 - protein, 7 aa, Artificial Sequence
  CDR_H3_3 of an anti-Ryk antibody or antibody derivative
HGDNGDY

SEQ ID NO: 9 - protein, 107 aa, Artificial Sequence
  Ab5.5_VL, a light chain variable region of an anti-Ryk antibody or antibody derivative
DIKMTQSPSSMYASLGERVTITCKASQDINSYLSWIQQKPGKSPKTLIYRANRLVDGVPSRFSGSGSGQDYSLTISSLEYEDMGIYYCLQYDEFPLTFGAGTKLELK

SEQ ID NO: 10 - protein, 116 aa, Artificial Sequence
  Ab5.5_VH, a heavy chain variable region of an anti-Ryk antibody or antibody derivative
EVKLVESGGDLVQPGGSLKLSCAASGFTFSSYTMSWIRQTPEKRLEWVAYISNGGGGTYYPDTVKGRFTISRDNAKNTLYLQMNSLKSEDTAMYYCTRHGDNGDYWGHGSTLTVSS

SEQ ID NO: 11 - protein, 107 aa, Artificial Sequence
  Ab5.5_VL_1, a light chain variable region of an anti-Ryk antibody or antibody derivative
DIQMTQSPSSLSASVGDRVTITCKASQDINSYLSWIQQKPGKAPKTLIYRANRLVDGVPSRFSGSGSGTDYTLTISSLQPEDFATYYCLQYDEFPLTFGAGTKVEIK

SEQ ID NO: 12 - protein, 107 aa, Artificial Sequence
  Ab5.5_VL_2, a light chain variable region of an anti-Ryk antibody or antibody derivative
DIQMTQSPSSLSASVGDRATITCKASQDINSYLSWIQQKPGKAPKTLIYRANRLVDGVPSRFSGSGSGTDYTLTISSLQPEDFATYYCLQYDEFPLTFGGGTKVEIK

SEQ ID NO: 13 - protein, 107 aa, Artificial Sequence
  Ab5.5_VL_3, a light chain variable region of an anti-Ryk antibody or antibody derivative
DIQMTQSPSSLSASVGDRATITCKASQDINSYLSWIQQKPGKAPKTLIYRANRLVEGVPSRFSGSGSGTDYTLTISSLQPEDFATYYCLQYDEFPLTFGGGTKVEIK

SEQ ID NO: 14 - protein, 116 aa, Artificial Sequence
  Ab5.5_VH_1, a heavy chain variable region of an anti-Ryk antibody or antibody derivative
EVQLVESGGGLVQPGGSLRLSCAASGFTFSSYTMSWIRQAPGKGLEWVAYISNGGGGTYYPDSVKGRFTISRDNAKNTLYLQMNSLRAEDTAVYYCTRHGDNGDYWGHGSLVTVSS

SEQ ID NO: 15 - protein, 116 aa, Artificial Sequence
  Ab5.5_VH_2, a heavy chain variable region of an anti-Ryk antibody or antibody derivative
EVQLVESGGGLVQPGGSLRLSCAASGFTFSSYTMSWIRQAPGKGLEWVAYISNGGGGTYYPDSVKGRFTISRDNAKNSLYLQMNSLRAEDTAVYYCTRHGDNGDYWGQGSLVTVSS

SEQ ID NO: 16 - protein, 116 aa, Artificial Sequence
  Ab5.5_VH_3, a heavy chain variable region of an anti-Ryk antibody or antibody derivative
EVQLVESGGGLVQPGGSLRLSCAASGFTFSSYTMSWIRQAPGKGLEWVAYISTGGGGTYYPDSVKGRFTISRDNAKNSLYLQMNSLRAEDTAVYYCTRHGDSGDYWGHGSLVTVSS

SEQ ID NO: 17 - protein, 116 aa, Artificial Sequence
  Ab5.5_VH_4, a heavy chain variable region of an anti-Ryk antibody or antibody derivative
EVQLVESGGGLVQPGGSLRLSCAASGFTFSSYTMSWIRQAPGKGLEWVAYISTGGGGTYYPDSVKGRFTISRDNAKNSLYLQMNSLRAEDTAVYYCTRHGDQGDYWGHGSLVTVSS

SEQ ID NO: 18 - protein, 116 aa, Artificial Sequence
  Ab5.5_VH_5, a heavy chain variable region of an anti-Ryk antibody or antibody derivative
EVQLVESGGGLVQPGGSLRLSCAASGFTFSSYTMSWIRQAPGKGLEWAAYISTGGGGTYYVDSVKGRFTISRDNAKNSLYLQMNSLRAEDTAVYYCTRHGDNGDYWGHGSLVTVSS

SEQ ID NO: 19 - protein, 8 aa, Homo sapiens
  PEPTIDE (1)..(8): Homo sapiens Ryk(211-218)
SRTIYDPV

SEQ ID NO: 20 - protein, 8 aa, Artificial Sequence
  Ryk artificial peptide 1
ARTIYDPV

SEQ ID NO: 21 - protein, 8 aa, Artificial Sequence
  Ryk artificial peptide 2
PRTIYDPV

SEQ ID NO: 22 - protein, 8 aa, Artificial Sequence
  Ryk artificial peptide 3
SRTLYDPV

SEQ ID NO: 23 - protein, 8 aa, Artificial Sequence
  Ryk artificial peptide 4
  MISC_FEATURE (3)..(3): X is a natural amino acid that is not T
SRXIYDPV

SEQ ID NO: 24 - protein, 594 aa, Mus musculus
  PEPTIDE (1)..(594): Mus musculus Ryk
MRAGRGGVPGSGGLRAPPPPLLLLLLAMLPAAAPRSPALAAAPAGPSVSLYLSEDEVRRLLGLDAELYYVRNDLISHYALSFNLLVPSETNFLHFTWHAKSKVEYKLGFQVDNFVAMGMPQVNISAQGEVPRTLSVFRVELSCTGKVDSEVMILMQLNLTVNSSKNFTVLNFKRRKMCYKKLEEVKTSALDKNTSRTIYDPVHAAPTTSTRVFYISVGVCCAVIFLVAIILAVLHLHSMKRIELDDSISASSSSQGLSQPSTQTTQYLRADTPNNATPITSSSGYPTLRIEKNDLRSVTLLEAKAKVKDIAISRERITLKDVLQEGTFGRIFHGILVDEKDPNKEKQTFVKTVKDQASEVQVTMMLTESCKLRGLHHRNLLPITHVCIEEGEKPMVVLPYMNWGNLKLFLRQCKLVEANNPQAISQQDLVHMAIQIACGMSYLARREVIHRDLAARNCVIDDTLQVKITDNALSRDLFPMDYHCLGDNENRPVRWMALESLVNNEFSSASDVWAFGVTLWELMTLGQTPYVDIDPFEMAAYLKDGYRIAQPINCPDELFAVMACCWALDPEERPKFQQLVQCLTEFHAALGAYV

SEQ ID NO: 25 - protein, 607 aa, Homo sapiens
  PEPTIDE (1)..(607): Homo sapiens Ryk
MRGAARLGRPGRSCLPGARGLRAPPPPPLLLLLALLPLLPAPGAAAAPAPRPPELQSASAGPSVSLYLSEDEVRRLIGLDAELYYVRNDLISHYALSFSLLVPSETNFLHFTWHAKSKVEYKLGFQVDNVLAMDMPQVNISVQGEVPRTLSVFRVELSCTGKVDSEVMILMQLNLTVNSSKNFTVLNFKRRKMCYKKLEEVKTSALDKNTSRTIYDPVHAAPTTSTRVFYISVGVCCAVIFLVAIILAVLHLHSMKRIELDDSISASSSSQGLSQPSTQTTQYLRADTPNNATPITSYPTLRIEKNDLRSVTLLEAKGKVKDIAISRERITLKDVLQEGTFGRIFHGILIDEKDPNKEKQAFVKTVKDQASEIQVTMMLTESCKLRGLHHRNLLPITHVCIEEGEKPMVILPYMNWGNLKLFLRQCKLVEANNPQAISQQDLVHMAIQIACGMSYLARREVIHKDLAARNCVIDDTLQVKITDNALSRDLFPMDYHCLGDNENRPVRWMALESLVNNEFSSASDVWAFGVTLWELMTLGQTPYVDIDPFEMAAYLKDGYRIAQPINCPDELFAVMACCWALDPEERPKFQQLVQCLTEFHAALGAYV

SEQ ID NO: 26 - protein, 94 aa, Homo sapiens
  PEPTIDE (1)..(94): Homo sapiens Ryk(134-227)
DMPQVNISVQGEVPRTLSVFRVELSCTGKVDSEVMILMQLNLTVNSSKNFTVLNFKRRKMCYKKLEEVKTSALDKNTSRTIYDPVHAAPTTSTR

SEQ ID NO: 27 - protein, 7 aa, Artificial Sequence
  Neutral peptide linker
GSGSGSG

SEQ ID NO: 28 - protein, 9 aa, Homo sapiens
  PEPTIDE (1)..(9): Homo sapiens Ryk(210-218)
TSRTIYDPV

SEQ ID NO: 29 - protein, 14 aa, Homo sapiens
  PEPTIDE (1)..(14): Homo sapiens Ryk(179-192)
SSKNFTVLNFKRRK

SEQ ID NO: 30 - protein, 14 aa, Homo sapiens
  PEPTIDE (1)..(14): Homo sapiens Ryk(184-197)
TVLNFKRRKMCYKK

SEQ ID NO: 31 - protein, 13 aa, Homo sapiens
  PEPTIDE (1)..(13): Homo sapiens Ryk(190-202)
RRKMCYKKLEEVK

SEQ ID NO: 32 - protein, 15 aa, Homo sapiens
  PEPTIDE (1)..(15): Homo sapiens Ryk(206-220)
LDKNTSRTIYDPVHA

SEQ ID NO: 33 - protein, 10 aa, Artificial Sequence
  Neutral peptide linker
YPYDVPDYAG

SEQ ID NO: 34 - protein, 5 aa, Homo sapiens
  PEPTIDE (1)..(5): Homo sapiens Ryk(206-210)
LDKNT

SEQ ID NO: 35 - protein, 15 aa, Homo sapiens
  PEPTIDE (1)..(15): Homo sapiens Ryk(178-192)
NSSKNFTVLNFKRRK

SEQ ID NO: 36 - protein, 13 aa, Homo sapiens
  PEPTIDE (1)..(13): Homo sapiens Ryk(185-197)
VLNFKRRKMCYKK

(19) United States
(12) Patent Application Publication
(10) Pub. No.: US 2023/0416375 A1
(43) Pub. Date: Dec. 28, 2023

(54) ANTIBODY VARIANTS AGAINST WNT RECEPTOR RYK

(71) Applicant: VERSAPEUTICS, INC., San Diego, CA (US)

(72) Inventors: David N. HAUSER, San Diego, CA (US); Taylor L. KAMPERT, San Diego, CA (US); Miao SUN, San Diego, CA (US)

(73) Assignee: VERSAPEUTICS, INC., San Diego, CA (US)

(21) Appl. No.: 18/035,407

(22) PCT Filed: Nov. 8, 2021
(86) PCT No.: PCT/US2021/058491

§ 371 Date: May 4, 2023

Related U.S. Application Data
(60) Provisional application No. 63/112,616, filed on Nov. 11, 2020.

(51) Int. Cl. C07K 16/28

(52) U.S. Cl. CPC C07K 16/2863; C07K 2317/24; C07K 2317/76; C07K 2317/34; C07K 2317/92; C07K 2317/732

(57) ABSTRACT

The present disclosure relates to isolated anti-Ryk antibodies or antibody derivatives. In some aspects, the present disclosure relates to the use of the isolated anti-Ryk antibodies or antibody derivatives.

### RELATED APPLICATIONS

The present application claims priority to U.S. provisional patent application No. 63/112,616, filed on Nov. 11, 2020, the disclosure and content of which is incorporated herein by reference in its entirety for all purposes.

### SEQUENCING LISTING ON ASCII TEXT

This patent or application file contains a Sequence Listing submitted in computer readable ASCII text format (file name: 4894-3000200 SeqList ST25.txt, date recorded: Oct. 27, 2020, size: 28,469 bytes). The content of the Sequence Listing file is incorporated herein by reference in its entirety.

### FIELD OF THE INVENTION

The present disclosure relates to isolated anti-Ryk antibodies or antibody derivatives. In some aspects, the present disclosure relates to the use of the isolated anti-Ryk antibodies or antibody derivatives.

### BACKGROUND

The Wnts are a family of secreted glycoproteins that bind to receptors on the cell surface and control a variety of cellular functions. The different pathways activated by the Wnts are divided into the canonical and non-canonical Wnt signaling pathways (Niehrs 2012). In the canonical pathway, Wnts bind to a complex consisting of a member of family of Frizzled receptor proteins and a co-receptor such as LRP5 or LRP6. The major downstream event in the canonical Wnt signaling is the stabilization of β-catenin, which results in changes in gene transcription that are critical for embryonic development and adult tissue homeostasis. Non-canonical Wnt signaling can be divided into the Wnt planar cell polarity (Wnt/PCP) and the Wnt/Ca 2+pathways that involve Wnts binding to a Frizzled family member and a co-receptor such as Ryk, PTK7, or ROR1/2. Signaling through the PCP pathway remodels the actin cytoskeleton and regulates cell migration and tissue organization.

Ryk is a single-pass transmembrane protein with a Wnt-inhibitory-factor-1 (WIF1) domain in its extracellular region that binds to Wnts (Patthy 2000). The intracellular region of Ryk contains a pseudokinase domain that has an inaccessible ATP-binding pocket and an inactive conformation (Sheetz et al. 2020). The intracellular C-terminus of Ryk contains a PDZ domain that is important for interactions with other proteins such as the Src family of kinases (Petrova et al. 2013). The downstream events that occur after Wnts bind to Ryk are not thoroughly described, but are thought to involve protein-protein interactions, signal transduction pathways, and proteolytic processing of Ryk (Roy, Halford, and Stacker 2018).

During embryonic development, Ryk is an important mediator of Wnt signaling in the central nervous system (Clark, Liu, and Cooper 2014). Axons expressing Ryk are repelled away from areas containing high concentrations of Wnt proteins, and this mechanism is critical in correctly establishing the corticospinal tract, corpus collosum, and retinotectal system (Schmitt et al. 2006; Y. Liu et al. 2005; Keeble et al. 2006). The functions of Ryk in normal adult tissues are less well understood, but Ryk is known to be involved in mammary stem/progenitor cell regulation as well as hematopoietic stem cell proliferation (Kessenbrock et al. 2017; Famili et al. 2016).

In addition to its normal biological functions, Ryk has a detrimental role in several pathologies. Following a spinal cord injury, several Wnts and Ryk are induced at the site of the injury and limit axon regeneration (Y. Liu et al. 2008; Hollis et al. 2016; Miyashita et al. 2009). Likewise, Wnts and Ryk are induced in injured spinal nerves and mediate the persistent hypersensitivity to painful stimuli following injury termed neuropathic pain (Zhang et al. 2013; S. Liu et al. 2015; Yang et al. 2017; Simonetti and Kuner 2020). High expression of Ryk occurs in several types of cancer, and Wnt/Ryk signaling plays a role in oncogenic processes such as tumor migration, invasiveness, and metastasis (VanderVorst et al. 2019; Roy, Halford, and Stacker 2018; Daulat and Borg 2017).

Anti-Ryk antibodies have been shown to promote axon regeneration after spinal cord injury and also to reduce neuropathic pain in rodent models (Hollis et al. 2016; Miyashita et al. 2009; S. Liu et al. 2015; Yang et al. 2017). However, murine and other non-human antibodies frequently elicit a strong immune response in humans (Khazaeli, Corny, and LoBuglio 1994), which limits their potential to be used as therapeutics. Therefore, there is a need for improved anti-Ryk antibodies with low immunogenic potential that can be developed to be used for the treatment of human pathologies including spinal cord injuries, neuropathic pain, and cancer. The present disclosure addresses this and the related needs. The references cited in the Background Section are listed below.
Clark, Charlotte E. J., Yaobo Liu, and Helen M. Cooper. 2014. “The Yin and Yang of Wnt/Ryk Axon Guidance in Development and Regeneration.” Science China. Life Sciences 57 (4): 366-71. https://doi.org/10.1007/s11427-014-4640-3.
Daulat, Avais M., and Jean-Paul Borg. 2017. “Wnt/Planar Cell Polarity Signaling: New Opportunities for Cancer Treatment.” Trends in Cancer 3 (2): 113-25. https://doi.org/10.1016/j.trecan.2017.01.001.
Famili, Farbod, Laura Garcia Perez, Brigitta Ae Naber, Jasprina N. Noordermeer, Lee G. Fradkin, and Frank Jt Staal. 2016. “The Non-Canonical Wnt Receptor Ryk Regulates Hematopoietic Stem Cell Repopulation in Part by Controlling Proliferation and Apoptosis.” Cell Death & Disease 7 (11): e2479. https://doi.org/10.1038/cddis.2016.380.
Hollis, Edmund R., Nao Ishiko, Ting Yu, Chin-Chun Lu, Ariela Haimovich, Kristine Tolentino, Alisha Richman, et al. 2016. “Ryk Controls Remapping of Motor Cortex during Functional Recovery after Spinal Cord Injury.” Nature Neuroscience 19 (5): 697-705. https://doi.org/10.1038/nn.4282.
Keeble, Thomas R., Michael M. Halford, Clare Seaman, Nigel Kee, Maria Macheda, Richard B. Anderson, Steven A. Stacker, and Helen M. Cooper. 2006. “The Wnt Receptor Ryk Is Required for Wnt5a-Mediated Axon Guidance on the Contralateral Side of the Corpus Callosum.” The Journal of Neuroscience: The Official Journal of the Society for Neuroscience 26 (21): 5840-48. https://doi.org/10.1523/JNEUROSCI.1175-06.2006.
Kessenbrock, Kai, Prestina Smith, Sander Christiaan Steenbeek, Nicholas Pervolarakis, Raj Kumar, Yasuhiro Minami, Andrei Goga, Lindsay Hinck, and Zena Werb. 2017. “Diverse Regulation of Mammary Epithelial Growth and Branching Morphogenesis through Noncanonical Wnt Signaling.” Proceedings of the National Academy of Sciences of the United States of America 114 (12): 3121-26. https://doi.org/10.1073/pnas.1701464114.
Khazaeli, M. B., R. M. Conry, and A. F. LoBuglio. 1994. “Human Immune Response to Monoclonal Antibodies.” Journal of Immunotherapy with Emphasis on Tumor Immunology: Official Journal of the Society for Biological Therapy 15 (1): 42-52. https://doi.org/10.1097/00002371-199401000-00006.
Liu, Su, Yue-Peng Liu, Zhi-Jiang Huang, Yan-Kai Zhang, Angela A. Song, Ping-Chuan Ma, and Xue-Jun Song. 2015. “Wnt/Ryk Signaling Contributes to Neuropathic Pain by Regulating Sensory Neuron Excitability and Spinal Synaptic Plasticity in Rats.” Pain 156 (12): 2572-84. https://doi.org/10.1097/j.pain. 0000000000000366.
Liu, Yaobo, Jun Shi, Chin-Chun Lu, Zheng-Bei Wang, Anna I. Lyuksyutova, Xue-Jun Song, Xuejun Song, and Yimin Zou. 2005. “Ryk-Mediated Wnt Repulsion Regulates Posterior-Directed Growth of Corticospinal Tract.” Nature Neuroscience 8 (9): 1151-59. https://doi.org/10.1038/nn1520.
Liu, Yaobo, Xiaofei Wang, Chin-Chun Lu, Rachel Kerman, Oswald Steward, Xiao-Ming Xu, and Yimin Zou. 2008. “Repulsive Wnt Signaling Inhibits Axon Regeneration after CNS Injury.” The Journal of Neuroscience: The Official Journal of the Society for Neuroscience 28 (33): 8376-82. https://doi.org/10.1523/JNEUROSCI.1939-08.2008.
Miyashita, Tomohiro, Masao Koda, Keiko Kitajo, Masashi Yamazaki, Kazuhisa Takahashi, Akira Kikuchi, and Toshihide Yamashita. 2009. “Wnt-Ryk Signaling Mediates Axon Growth Inhibition and Limits Functional Recovery after Spinal Cord Injury.” Journal of Neurotrauma 26 (7): 955-64. https://doi.org/10.1089/neu.2008.0776.
Niehrs, Christof. 2012. “The Complex World of WNT Receptor Signalling.” Nature Reviews Molecular Cell Biology 13 (12): 767-79. https://doi.org/10.1038/nrm3470.
Patthy, L. 2000. “The WIF Module.” Trends in Biochemical Sciences 25 (1): 12-13. https://doi.org/10.1016/s0968-0004(99)01504-2.
Petrova, Iveta M., Liza L. Lahaye, Tania Martiáñez, Anja W. M. de Jong, Martijn J. Malessy, Joost Verhaagen, Jasprina N. Noordermeer, and Lee G. Fradkin. 2013. “Homodimerization of the Wnt Receptor DERAILED Recruits the Src Family Kinase SRC64B.” Molecular and Cellular Biology 33 (20): 4116-27. https://doi.org/10.1128/MCB.00169-13.
Roy, James P., Michael M. Halford, and Steven A. Stacker. 2018. “The Biochemistry, Signalling and Disease Relevance of RYK and Other WNT-Binding Receptor Tyrosine Kinases.” Growth Factors 36 (1-2): 15-40. https://doi.org/10.1080/08977194.2018.1472089.
Schmitt, Adam M., Jun Shi, Alex M. Wolf, Chin-Chun Lu, Leslie A. King, and Yimin Zou. 2006. “Wnt-Ryk Signalling Mediates Medial-Lateral Retinotectal Topographic Mapping.” Nature 439 (7072): 31-37. https://doi.org/10.1038/nature04334.
Sheetz, Joshua B., Sebastian Mathea, Hanna Karvonen, Ketan Malhotra, Deep Chatterjee, Wilhelmiina Niininen, Robert Perttilä, et al. 2020. “Structural Insights into Pseudokinase Domains of Receptor Tyrosine Kinases.” Molecular Cell 79 (3): 390-405.e7. https://doi.org/10.1016/j.molcel.2020.06.018.
Simonetti, Manuela, and Rohini Kuner. 2020. “Spinal Wnt5a Plays a Key Role in Spinal Dendritic Spine Remodeling in Neuropathic and Inflammatory Pain Models and in the Pro-Algesic Effects of Peripheral Wnt3a.” The Journal of Neuroscience: The Official Journal of the Society for Neuroscience, July. https://doi.org/10.1523/JNEUROSCI.2942-19.2020.
VanderVorst, Kacey, Courtney A. Dreyer, Sara E. Konopelski, Hyun Lee, Hsin-Yi Henry Ho, and Kermit L. Carraway. 2019. “Wnt/PCP Signaling Contribution to Carcinoma Collective Cell Migration and Metastasis.” Cancer Research 79 (8): 1719-29. https://doi.org/10.1158/0008-5472. CAN-18-2757.
Yang, Qing Ou, Wen-Jing Yang, Jian Li, Fang-Ting Liu, Hongbin Yuan, and Yue-Ping Ou Yang. 2017. “Ryk Receptors on Unmyelinated Nerve Fibers Mediate Excitatory Synaptic Transmission and CCL2 Release during Neuropathic Pain Induced by Peripheral Nerve Injury.” Molecular Pain 13 (May). https://doi.org/10.1177/1744806917709372.
Zhang, Yan-Kai, Zhi-Jiang Huang, Su Liu, Yue-Peng Liu, Angela A. Song, and Xue-Jun Song. 2013. “WNT Signaling Underlies the Pathogenesis of Neuropathic Pain in Rodents.” The Journal of Clinical Investigation 123 (5): 2268-86. https://doi.org/10.1172/JCI65364.

### SUMMARY OF THE INVENTION

In one aspect or embodiment, the present disclosure provides for an isolated anti-Ryk antibody or antibody derivative that: a) specifically binds to a Wnt-binding domain on Ryk or specifically binds to an epitope within a region of the ectodomain of Ryk, e.g., amino-acids 35-211 of mouse Ryk (SEQ ID NO:24) or amino-acids 26-227 of human Ryk (SEQ ID NO:25), the antibody or antibody derivative comprising a light chain variable region comprising the CDR sequence set forth in SEQ ID NO:1 [RANRLVE]; b) specifically binds to the same epitope on a Wnt-binding domain on Ryk or the same epitope within a region of the ectodomain of Ryk, e.g., amino-acids 35-211 of mouse Ryk (SEQ ID NO:24) or amino-acids 26-227 of human Ryk (SEQ ID NO:25), as does a reference antibody or antibody derivative, or cross-competes for specific binding to the Wnt-binding domain on Ryk or the same epitope within a region of the ectodomain of Ryk with a reference antibody or antibody derivative, the reference antibody or antibody derivative comprising a light chain variable region comprising the CDR sequence set forth in SEQ ID NO:1 [RANRLVE]; c) specifically binds to a Wnt-binding domain on Ryk or specifically binds to an epitope within a region of the ectodomain of Ryk, e.g., amino-acids 35-211 of mouse Ryk (SEQ ID NO:24) or amino-acids 26-227 of human Ryk (SEQ ID NO:25), said antibody or antibody derivative comprising a heavy chain variable region comprising the CDR sequence set forth in SEQ ID NO:2 [STGGGGTY], SEQ ID NO:3 [HGDSGDY] or SEQ ID NO:4 [HGDQGDY]; and/or d) specifically binds to the same epitope on a Wnt-binding domain on Ryk or the same epitope within a region of the ectodomain of Ryk, e.g., amino-acids of mouse Ryk (SEQ ID NO:24) or amino-acids 26-227 of human Ryk (SEQ ID NO:25), as does a reference antibody or antibody derivative, or cross-competes for specific binding to the Wnt-binding domain on Ryk or the same epitope within a region of the ectodomain of Ryk with a reference antibody or antibody derivative, the reference antibody or antibody derivative comprising a heavy chain variable region comprising the CDR sequence set forth in SEQ ID NO:2 [STGGGGTY], SEQ ID NO:3 [HGDSGDY] or SEQ ID NO:4 [HGDQGDY], provided that the antibody or antibody derivative is not an isolated anti-Ryk antibody or antibody derivative disclosed and/or claimed in WO 2017/172733 A1.

In another aspect or embodiment, the present disclosure provides for an isolated anti-Ryk antibody or antibody derivative that: a) specifically binds to a Wnn-binding domain on Ryk or specifically binds to an epitope within a region of the ectodomain of Ryk, e.g., amino-acids of mouse Ryk (SEQ ID NO:24) or amino-acids 26-227 of human Ryk (SEQ ID NO:25), the antibody or antibody derivative comprises a light chain variable region comprising an amino acid sequence comprising at least about 85% sequence identity to SEQ ID NO:11 [VL1], SEQ ID NO:12 [VL2], or SEQ ID NO:13 [VL3]; b) specifically binds to the same epitope on a Wnt-binding domain on Ryk or the same epitope within a region of the ectodomain of Ryk, e.g., amino-acids 35-211 of mouse Ryk (SEQ ID NO:24) or amino-acids 26-227 of human Ryk (SEQ ID NO:25), as does a reference antibody or antibody derivative, or cross-competes for specific binding to the Wnt-binding domain on Ryk or the same epitope within a region of the ectodomain of Ryk with a reference antibody or antibody derivative, the reference antibody or antibody derivative comprising a light chain variable region comprising an amino acid sequence comprising at least about 85% sequence identity to SEQ ID NO:11 [VL1], SEQ ID NO:12 [VL2], or SEQ ID NO:13 [VL3]; c) specifically binds to a Wnt-binding domain on Ryk or specifically binds to an epitope within a region of the ectodomain of Ryk, e.g., amino-acids 35-211 of mouse Ryk (SEQ ID NO:24) or amino-acids 26-227 of human Ryk (SEQ ID NO:25), the antibody or antibody derivative comprises a heavy chain variable region that comprises an amino acid sequence comprising at least about 85% sequence identity to SEQ ID NO:14 [VH1], SEQ ID NO:15 [VH2], SEQ ID NO:16 [VH3], SEQ ID NO:17 [VH4], or SEQ ID NO:18 [VH5]; and/or d) specifically binds to the same epitope on a Wnt-binding domain on Ryk or the same epitope within a region of the ectodomain of Ryk, e.g., amino-acids 35-211 of mouse Ryk (SEQ ID NO:24) or amino-acids 26-227 of human Ryk (SEQ ID NO:25), as does a reference antibody or antibody derivative, or cross-competes for specific binding to the Wnt-binding domain on Ryk or the same epitope within a region of the ectodomain of Ryk with a reference antibody or antibody derivative, the reference antibody or antibody derivative comprising a heavy chain variable region that comprises an amino acid sequence comprising at least about 85% sequence identity to SEQ ID NO:14 [VH1], SEQ ID NO:15 [VH2], SEQ ID NO:16 [VH3], SEQ ID NO:17 [VH4], or SEQ ID NO:18 [VH5], provided that the antibody or antibody derivative is not an isolated anti-Ryk antibody or antibody derivative disclosed and/or claimed in WO 2017/172733 A1.

In still another aspect or embodiment, the present disclosure provides for an immunoconjugate comprising the above isolated antibody or antibody derivative, linked to a detecting and/or therapeutic agent.

In yet another aspect or embodiment, the present disclosure provides for a bispecific molecule comprising the above isolated antibody or antibody derivative, linked to a second functional moiety having a different binding specificity than the above isolated antibody or antibody derivative.

In yet another aspect or embodiment, the present disclosure provides for a pharmaceutical composition comprising an effective amount of the above antibody or antibody derivative, the above immunoconjugate, or the above bispecific molecule, and a pharmaceutically acceptable carrier or excipient.

In yet another aspect or embodiment, the present disclosure provides for a nucleic acid sequence encoding the above isolated antibody or antibody derivative. A vector comprising the above nucleic acid sequence is also provided. A host cell comprising the above vector is further provided. A transgenic non-human animal, e.g., a transgenic mouse, comprising the above host cell, wherein the non-human animal or mouse expresses a polypeptide encoded by the above nucleic acid is further provided.

In yet another aspect or embodiment, the present disclosure provides for a method of interfering with interaction of Wnt and Ryk comprising contacting a sample comprising Wnt and Ryk with the above isolated antibody or antibody derivative, the above immunoconjugate, the above bispecific molecule, thereby interfering with the interaction of Wnt and Ryk.

In yet another aspect or embodiment, the present disclosure provides for a method for inhibiting degeneration of a neuron, the method comprising contacting the neuron with the above isolated antibody or antibody derivative, the above immunoconjugate, the above bispecific molecule, the above pharmaceutical composition, the above nucleic acid sequence, the above vector, or the above host cell, thereby inhibiting degeneration of the neuron.

In yet another aspect or embodiment, the present disclosure provides for a method of preventing or treating a neurological disease, disorder or injury in a subject having or being at risk of developing the neurological disease, disorder or injury comprising administering to the subject an effective amount of the above isolated antibody or antibody derivative, the above immunoconjugate, the above bispecific molecule, the above pharmaceutical composition, the above nucleic acid sequence, the above vector, or the above host cell, thereby treating the neurological disease, disorder or injury in the subject.

In yet another aspect or embodiment, the present disclosure provides for a method for modulating the directional growth of a neuron comprising contacting the neuron with the above isolated antibody or antibody, the above immunoconjugate, the above bispecific molecule, the above pharmaceutical composition, the above nucleic acid sequence, the above vector, or the above host cell, thereby modulating the directional growth of the neuron.

In yet another aspect or embodiment, the present disclosure provides for an use of an effective amount of the above isolated antibody or antibody derivative, the above immunoconjugate, the above bispecific molecule, the above nucleic acid sequence, the above vector, or the above host cell for manufacturing a medicament for treating or preventing a neurological disease, disorder or injury in a subject having or being at risk of developing the neurological disease, disorder or injury.

In yet another aspect or embodiment, the present disclosure provides for a method of preventing or treating a cancer or tumor in a subject having or being at risk of developing the cancer or tumor comprising administering to the subject an effective amount of the above isolated antibody or antibody derivative, the above immunoconjugate, the above bispecific molecule, the above pharmaceutical composition, the above nucleic acid sequence, the above vector, or the above host cell, thereby treating or treating the cancer or tumor in the subject.

In yet another aspect or embodiment, the present disclosure provides for an use of an effective amount of the above isolated antibody or antibody derivative, the above immunoconjugate, the above bispecific molecule, the above nucleic acid sequence, the above vector of, or the above host cell for manufacturing a medicament for preventing or treating a cancer or tumor in a subject having or being at risk of developing the cancer or tumor.

In yet another aspect or embodiment, the present disclosure provides for a method for assessing a Ryk polypeptide in a sample, which method comprises: a) contacting a sample containing or suspected of containing a Ryk polypeptide with the above isolated antibody or antibody derivative, the above immunoconjugate, or the above bispecific molecule; and b) assessing binding between the Ryk polypeptide, if present in the sample, and the above isolated antibody or antibody derivative, the above immunoconjugate or the above bispecific molecule to assess the presence, absence, level or amount of the Ryk polypeptide in the sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an exemplary Ab5.5 VL domain with Chothia CDR definitions and numbering.

FIG. 2 illustrates an exemplary Ab5.5 VH domain with Chothia CDR definitions and numbering.

FIG. 3 illustrates an exemplary alignment of Ab5.5 VL domain to the Acceptor framework.

FIG. 4 illustrates an exemplary alignment of Ab5.5 VH domain to the Acceptor framework.

FIG. 5 illustrates an exemplary global DRB 1 risk scores for Ab5.5 and the lowest DRB 1 scoring variant, Ab5.5 var15, against a histogram of the DRB 1 scores of 44 marketed therapeutic antibodies. Human antibodies are shown by blue bars, humanised by light blue bars and chimeric by dark blue bars. DRB 1 scores in the reference set have been predicted for antibody variable domains or complete antibodies.

FIG. 6 illustrates an exemplary epitope mapping for four antibody variants, Ab5.5, Ab5.5 earl, Ab5.5 var2, and Ab5.5 var10.

FIG. 7 illustrates an exemplary sequence alignment of the recombinant fusion proteins containing the human Ryk antigen sequence with (A, Antigen) and without (DE, Deleted Epitope) the putative epitope that was discovered using peptide mapping. The Maltose-binding protein (MBP) sequence is shown in orange , thrombin cleavage site is in blue, and human Ryk sequences are shown in green.

FIG. 8 illustrates an exemplary Western blot screen of Ab5.5 variants with recombinant human Ryk antigen. The Ab5.5 variants were used in an immunoblot to detect the human Ryk protein sequence that either did (A, Antigen) or did not (DE, Delta Epitope) have the putative epitope, which is amino acid sequence TSRTIYDPV. Both recombinant proteins were tagged with maltose-binding protein (MBP). Top panel shows immunoreactivity of the Ab5.5 variant and the bottom panel is the identical blot probed with an anti-MBP antibody. The graph shows the analysis of the band intensity of the Ab5.5 variant binding normalized to MBP (N=3 experiments).

FIG. 9 illustrates exemplary Ab5.5 Substitution Scan Heatmap.

FIG. 10 illustrates exemplary Ab5.5 Substitution Scan Amino Acid Plot.

FIG. 11 illustrates exemplary Ab5.5_var1 Substitution Scan Heatmap.

FIG. 12 illustrates exemplary Ab5.5_var1 Substitution Scan Amino Acid Plot.

FIG. 13 illustrates exemplary canonical Wnt signaling in HEK 293 STF cell is inhibited by Ab5.5_var1.

FIG. 14 illustrates exemplary RYK mRNA expression in cholangio carcinoma.

FIG. 15 illustrates exemplary Ryk mRNA expression in lymphoid neoplasm diffuse large B-cell lymphoma.

FIG. 16 illustrates exemplary Ryk mRNA expression in glioblastoma multiforme.

FIG. 17 illustrates exemplary Ryk mRNA expression in head and neck squamous cell carcinoma.

FIG. 18 illustrates exemplary Ryk mRNA expression in acute myeloid leukemia.

FIG. 19 illustrates exemplary Ryk mRNA expression in lower grade glioma.

FIG. 20 illustrates exemplary Ryk mRNA expression lung squamous cell carcinoma.

FIG. 21 illustrates exemplary Ryk mRNA expression in pancreatic adenocarcinoma.

FIG. 22 illustrates exemplary Ryk mRNA expression in thymoma.

FIG. 23 illustrates exemplary High Ryk mRNA levels are associated with poor survival in lower grade glioma.

FIG. 24 illustrates exemplary High Ryk mRNA levels are associated with poor survival in pancreatic adenocarcinoma.

FIG. 25 illustrates an exemplary Western blot binding confirmation of Ab5.5_Var1 with both full length human and mouse RYK that expressed in human HEK293 cell line. The vector that encodes human and mouse RYK construct was used as empty control.

FIG. 26 illustrates an exemplary function blocking of Ab5.5_Var1 to Wnt5a conducted SK-N-SH human neuroblastoma cell migration. The migrated cells were labeled by Hoechst (Blue) and the number of which was automatically quantified by Cytation 5 Cell Imaging Multi-Mode Reader. The graph shows the analysis of the number of migrated cells under indicated treatments normalized to non-treatment group (N=3 experiments).

FIG. 27 illustrates an exemplary cytotoxicity study conducted by combination treatment of Ab5.5_Var1 and αHFc-CL-PNU antibody, but not Ab5.5_Var1 alone nor IgG with αHFc-CL-PNU antibody. The graph shows the analysis of cell viability that is normalized to non-treatment group (N=3 experiments).

FIG. 28 illustrates an exemplary function blocking of Ab5.5_Var1 to Wnt5a induced RhoA activation in human HEK293 cell line. The graph shows the analysis of the percentage of induction that is normalized to the level of total RhoA (N=3 experiments).

### DETAILED DESCRIPTION

A detailed description of one or more embodiments of the claimed subject matter is provided below along with accompanying figures that illustrate the principles of the claimed subject matter. The claimed subject matter is described in connection with such embodiments, but is not limited to any particular embodiment. It is to be understood that the claimed subject matter may be embodied in various forms, and encompasses numerous alternatives, modifications and equivalents. Therefore, specific details disclosed herein are not to be interpreted as limiting, but rather as a basis for the claims and as a representative basis for teaching one skilled in the art to employ the claimed subject matter in virtually any appropriately detailed system, structure, or manner. Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

All publications, including patent documents, scientific articles and databases, referred to in this application are incorporated by reference in their entireties for all purposes to the same extent as if each individual publication were individually incorporated by reference. If a definition set forth herein is contrary to or otherwise inconsistent with a definition set forth in the patents, patent applications, published applications or other publications that are herein incorporated by reference, the definition set forth herein prevails over the definition that is incorporated herein by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

The practice of the provided embodiments will employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polypeptide and protein synthesis and modification, polynucleotide synthesis and modification, polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press; A. Bothwell et al. eds., Methods for Cloning and Analysis of Eukaryotic Genes (1990), Bartlett Publ.; M. Kriegler, Gene Transfer and Expression (1990), Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W.H. Freeman Pub., New York, N.Y.; Berg, et al., Biochemistry (2002) 5th Ed., W. H. Freeman Pub., New York, N.Y.; D. Weir & C. Blackwell, eds., Handbook of Experimental Immunology (1996), Wiley-Blackwell; Cellular and Molecular Immunology (A. Abbas et al., W.B. Saunders Co. 1991, 1994); Current Protocols in Immunology (J. Coligan et al. eds. 1991), all of which are herein incorporated in their entireties by reference for all purposes.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

In some embodiments, The present invention is based on the finding that an anti-Ryk antibody or antibody fragment that specifically binds to a binding domain of Wnt on Ryk inhibits Wnt-Ryk signaling. In some embodiments, the present invention provides methods for modulating neuron degeneration and neuron guidance using the anti-Ryk antibody or antibody fragment. Thus, the anti-Ryk antibody or antibody fragment can be used to treat a neurological disease or disorder, e.g., a neurodegenerative disease or disorder, in a subject having or being at risk of developing the neurological disease or disorder, e.g., a neurodegenerative disease or disorder, and/or to treat spinal cord injury (SCI) in a subject.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

A. Definitions

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a composition refers to any mixture of two or more products, substances, or compounds, including cells. It may be a solution, a suspension, liquid, powder, a paste, aqueous, non-aqueous or any combination thereof

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ϵ, γ, and μ, respectively.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745.” (“Contact” numbering scheme), Lefranc MP et al., “IMGT unique numbering for immunoglobulin and T cell receptor variable domains and Ig superfamily V-like domains,” Dev Comp Immunol, 2003 January;27(1):55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8;309(3):657-70, (“Aho” numbering scheme).

The boundaries of a given CDR or FR may vary depending on the scheme used for identification. For example, the Kabat scheme is based structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Thus, unless otherwise specified, a “CDR” or “complementary determining region,” or individual specified CDRs (e.g., “CDR-H1, CDR-H2), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given V_{H} or V_{L} amino acid sequence, it is understood that such a CDR has a sequence of the corresponding CDR (e.g., CDR-H3) within the variable region, as defined by any of the aforementioned schemes.

Likewise, unless otherwise specified, a FR or individual specified FR(s) (e.g., FR-H1, FR-H2), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, or Contact method.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. See, e.g., Kindt et al., Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. In particular embodiments, the antibodies are single-chain antibody fragments comprising a variable heavy chain region and/or a variable light chain region, such as scFvs.

Single-domain antibodies are antibody fragments comprising all or a portion of the heavy chain variable domain or all or a portion of the light chain variable domain of an antibody. In certain embodiments, a single-domain antibody is a camelid single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody.

A “humanized” antibody is an antibody in which all or substantially all CDR amino acid residues are derived from non-human CDRs and all or substantially all FR amino acid residues are derived from human FRs. The term “chimeric” antibody refers to an antibody in which a portion of the heavy and/or light chain is derived from a particular source or species, while the remainder of the heavy and/or light chain is derived from a different source or species.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

The terms “polypeptide” and “protein” are used interchangeably to refer to a polymer of amino acid residues, and are not limited to a minimum length. Polypeptides, including the provided antibodies and antibody chains and other peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

As used herein, the term “specific binding” refers to the specificity of a binder, e.g., an antibody, such that it preferentially binds to a target, such as a polypeptide antigen. When referring to a binding partner, e.g., protein, nucleic acid, antibody or other affinity capture agent, etc., “specific binding” can include a binding reaction of two or more binding partners with high affinity and/or complementarity to ensure selective hybridization under designated assay conditions. Typically, specific binding will be at least three times the standard deviation of the background signal. Thus, under designated conditions the binding partner binds to its particular target molecule and does not bind in a significant amount to other molecules present in the sample. Recognition by a binder or an antibody of a particular target in the presence of other potential interfering substances is one characteristic of such binding. Preferably, binders, antibodies or antibody fragments that are specific for or bind specifically to a target bind to the target with higher affinity than binding to other non-target substances. Also preferably, binders, antibodies or antibody fragments that are specific for or bind specifically to a target avoid binding to a significant percentage of non-target substances, e.g., non-target substances present in a testing sample. In some embodiments, binders, antibodies or antibody fragments of the present disclosure avoid binding greater than about 90% of non-target substances, although higher percentages are clearly contemplated and preferred. For example, binders, antibodies or antibody fragments of the present disclosure avoid binding about 91%, about 92%, about 93%, about 94%, about 95%, about 96%, about 97%, about 98%, about 99%, and about 99% or more of non-target substances. In other embodiments, binders, antibodies or antibody fragments of the present disclosure avoid binding greater than about 10%, 20%, 30%, 40%, 50%, 60%, or 70%, or greater than about 75%, or greater than about 80%, or greater than about 85% of non-target substances.

An “individual” or “subject” includes a mammal. Mammals include, but are not limited to, domesticated animals (e.g., cows, sheep, cats, dogs, and horses), primates (e.g., humans and non-human primates such as monkeys), rabbits, and rodents (e.g., mice and rats). An “individual” or “subject” may include birds such as chickens, vertebrates such as fish and mammals such as mice, rats, rabbits, cats, dogs, pigs, cows, ox, sheep, goats, horses, monkeys and other non-human primates. In certain embodiments, the individual or subject is a human.

As used herein, the term “sample” refers to anything which may contain an analyte for which an analyte assay is desired. As used herein, a “sample” can be a solution, a suspension, liquid, powder, a paste, aqueous, non-aqueous or any combination thereof. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

An “isolated” antibody is one which has been separated from a component of its natural environment. In some embodiments, an antibody is purified to greater than 90%, 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, “treatment’ or ‘treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The term “effective amount” or “therapeutically effective amount” refers to the amount of an active agent sufficient to induce a desired biological result. That result may be alleviation of the signs, symptoms, or causes of a disease, or any other desired alteration of a biological system. The term “therapeutically effective amount” is used herein to denote any amount of the formulation which causes a substantial improvement in a disease condition when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

A “subject,” “individual,” or “patient,” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vitro or cultured in vitro are also encompassed.

As used herein, “promote” or “increase,” or “promoting” or “increasing” are used interchangeably herein. These terms refer to the increase in a measured parameter (e.g., activity, expression, signal transduction, neuron degeneration) in a treated cell (tissue or subject) in comparison to an untreated cell (tissue or subject). A comparison can also be made of the same cell or tissue or subject between before and after treatment. The increase is sufficient to be detectable. In some embodiments, the increase in the treated cell is at least about 10%, 20%, 30%, 40%, 50%, 60%, 70%, 80%, 90%, 1-fold, 2-fold, 3-fold, 4-fold or more in comparison to an untreated cell.

As used herein, “inhibit,” “prevent” or “reduce,” or “inhibiting,” “preventing’ or “reducing’ are used interchangeably herein. These terms refer to the decrease in a measured parameter (e.g., activity, expression, signal transduction, neuron degeneration) in a treated cell (tissue or subject) in comparison to an untreated cell (tissue or subject). A comparison can also be made of the same cell or tissue or subject between before and after treatment. The decrease is sufficient to be detectable. In some embodiments, the decrease in the treated cell is at least about 10%, 20%, 30%, 40%, 50%, 60%, 70%, 80%, 90%, or completely inhibited in comparison to an untreated cell. In some embodiments the measured parameter is undetectable (i.e., completely inhibited) in the treated cell in comparison to the untreated cell.

The term “selective inhibition” or “selectively inhibit” as referred to a biologically active agent refers to the agent's ability to preferentially reduce the target signaling activity as compared to off-target signaling activity, via direct or indirect interaction with the target.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are exemplary conservative substitutions for one another: [to be added]

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. The invention provides polypeptides that are substantially identical to the polypeptides, respectively, exemplified herein, as well as uses thereof including, but not limited to, use for treating or preventing neurological diseases or disorders, e.g., neurodegenerative diseases or disorders, and/or treating SCI. Optionally, the identity exists over a region that is at least about 50 nucleotides in length, or more preferably over a region that is 100 to 500 or 1000 or more nucleotides in length, or the entire length of the reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-0-methyl ribonucleotides, peptide-nucleic acids (PNAs).

As used herein, the term “dominant negative mutant” of a protein refers to a mutant polypeptide or nucleic acid, which lacks wild-type activity and which, once expressed in a cell wherein a wild-type of the same protein is also expressed, dominates the wild-type protein and effectively competes with wild type proteins for substrates, ligands, etc., and thereby inhibits the activity of the wild type molecule. The dominant negative mutant can be a polypeptide having an amino acid sequence substantially similar (i.e., at least about 75%, about 80%, about 85%, about 90%, about 95% similar) to the wild type protein. The dominant negative mutant can also be a polypeptide comprising a fragment of the wild type protein, e.g., the C-domain of the wild-type protein. The dominant negative mutant can be a truncated form of the wild type protein.

Mouse Model

As used herein, “transgenic organism” refers to an animal in which exogenous DNA has been introduced while the animal is still in its embryonic stage. In most cases, the transgenic approach aims at specific modifications of the genome, e.g., by introducing whole transcriptional units into the genome, or by up- or down-regulating or mutating pre-existing cellular genes. The targeted character of certain of these procedures sets transgenic technologies apart from experimental methods in which random mutations are conferred to the germline, such as administration of chemical mutagens or treatment with ionizing solution. A transgenic organism can include an organism which has a gene knockout or may result for inducing a genetic mutation.

A “genetic knock out” refers to partial or complete suppression of the expression of a protein encoded by an endogenous DNA sequence in a cell. The “knockout” can be affected by targeted deletion of the whole or part of a gene encoding a protein. Alternatively, the transgenic organism can be obtained by the targeted mutation of a functional protein in an embryonic stem cell. As a result, the deletion or mutation may prevent or reduce the expression of the protein in any cell in the whole animal in which it is normally expressed, or results in the expression of a mutant protein having a biological function different than the normal/wild-type protein.

The term “knockout animal” and “transgenic animal”, refer to a transgenic animal wherein a given gene has been suppressed or mutated by recombination with a targeting vector. It is to be emphasized that the term is intended to include all progeny generations. Thus, the founder animal and all F1, F2, F3, and so on, progeny thereof are included.

As used herein, the phrase “conditional knockout,” or “cKO,” when used to describe a non-human transgenic mammal such as a mouse, refers to mice containing a knock-out of a specific gene in a certain tissue. The creation of a genetically engineered cKO mouse involves inserting specific DNA sequences, such as a knock-out construct/vector, into the mouse DNA. The inserted sequences are recognized by two DNA specific enzymes, frt recombinase (also known as flippase) and Cre recombinase, not normally present in mice. Cre recombinase recognition sites are termed loxP sites and flippase recognition sites are termed frt sites. Each of these enzymes can cut and remove a DNA sequence that is flanked by its recognitions sites. This can lead to disruption of gene function if a functional DNA sequence of the gene of interest is removed. In addition, a selectable marker gene is inserted into the mouse, the introduction of which allows selection of embryonic mouse cells (stem cells) that contain the Cre recombination or flippase recognition sites. The resultant mouse is a conditional knockout mouse.

A knock-out construct is a nucleic acid sequence, such as a DNA construct, which, when introduced into a cell, results in suppression (partial or complete) of expression of a polypeptide or protein encoded by endogenous DNA in the cell. An exemplary knock-out construct is provided herein. This construct contains a loxP site 5′ to exon 3 and 3′ to exon 6 of the Ryk gene, a selectable marker cassette and a loxP site 3′ to the selectable marker cassette. The selectable marker cassette comprises frt sites 5′ and 3′ to the selectable marker and is between the 3′ frt site and the selectable marker gene. Suitable selectable markers include, but are not limited to, neomycin, puromycin and hygromycin.

Animals containing more than one transgenic construct and/or more than one transgene expression construct may be prepared in any of several ways. An exemplary manner of preparation is to generate a series of animals, each containing one of the desired transgenic phenotypes. Such animals are bred together through a series of crosses, backcrosses and selections, to ultimately generate a single animal containing all desired transgenic traits and/or expression constructs, where the animal is otherwise congenic (genetically identical) to the wild type except for the presence of the construct(s) and/or transgene(s).

Embryonic stem (ES) cells are typically selected for their ability to integrate into and become part of the germ line of a developing embryo so as to create germ line transmission of the transgene. Thus, any ES cell line that can do so is suitable for use herein. ES cells are generated and maintained using methods well known to the skilled artisan, such as those described by Doetschman et al. (1985) J. Embryol. Exp. Mol. Biol. 87:27-45). Any line of ES cells can be used, however, the line chosen is typically selected for the ability of the cells to integrate into and become part of the germ line of a developing embryo so as to create germ line transmission of the transgenic/knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934). Still another ES cell line is the WW6 cell line (Ioffe et al. (1995) PNAS 92:7357-7361). The cells are cultured and prepared for knockout construct insertion using methods well known to the skilled artisan, such as those set forth by Robertson in: Teratocarcinomas and Embryonic Stem Cells: A Practical Approach, E. J. Robertson, ed. IRL Press, Washington, D.C. (1987)); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. (1986)).

Introduction of the knock-out construct into ES cells may be accomplished using a variety of methods well-known in the art, including, for example, electroporation, microinjection, and calcium phosphate treatment. For introduction of the DNA sequence, the knock-out construct DNA is added to the ES cells under appropriate conditions for the insertion method chosen. If the cells are to be electroporated, the ES cells and construct DNA are exposed to an electric pulse using an electroporation machine (electroporator) and following the manufacturer's guidelines for use. After electroporation, the cells are allowed to recover under suitable incubation conditions. The cells are then screened for the presence of the knockout construct. Screening for cells which contain the transgene (homologous recombinants) may be done using a variety of methods. For example, as described herein, cells can be processed as needed to render DNA in them available for screening with specific probes by polymerase chain reaction (PCR).

Once appropriate ES cells are identified, they are introduced into an embryo using standard methods. They can be introduced using microinjection, for example. Embryos at the proper stage of development for integration of the ES cell to occur are obtained, such as by perfusion of the uterus of pregnant females. For example, mouse embryos at 3-4 days development can be obtained and injected with ES cells using a micropipet. After introduction of the ES cell into the embryo, the embryo is introduced into the uterus of a pseudopregnant female mouse. The stage of the pseudopregnancy is selected to enhance the chance of successful implantation. In mice, 2-3 days pseudopregnant females are appropriate.

Successful incorporation of ES cells into implanted embryos results in offspring termed chimeras. Chimeras capable of germline transmission of the mutant allele are identified by standard methods. Chimeras are bred and the resulting progeny are screened for the presence of the desired alteration (e.g., the modified recombinant Ryk allele). This may be done, for example, on the basis of coat color or by obtaining DNA from offspring (e.g., tail DNA) to assess for the transgene, using known methods (e.g., Southern analysis, dot blot analysis, PCR analysis). Transgene expression may also be assessed (e.g., to determine if a replacement construct is expressed) by known methods, such as northern analysis or PCR analysis. Southern hybridization or PCR analysis of progeny DNA (e.g., tail DNA) may be conducted to identify desired genotypes. A suitable technique for obtaining completely ES cell derived transgenic non-human organisms is described in WO 98/06834, incorporated herein by reference.

In various embodiments, the cKO mice disclosed herein include at least three elements: (1) at least two enzyme-specific recognition sites flanking a critical portion of the target gene; (2) a gene encoding a selection marker such as, but not limited to neomycin; and (3) at least two enzyme-specific recognition sites flanking a selection marker gene for easy removal upon breeding with specific mouse strains. In a non-limiting example, exons 3-6 of the target gene has been designated as the critical portion. In one embodiment the enzyme-specific recognition sites flanking the critical portion of the target gene are loxP sites. In another embodiment, the enzyme-specific recognition sites flanking the selection marker gene are frt sites.

As mentioned above, the homologous recombination of the above described “knock-out” and/or “knock in” constructs is sometimes rare and such a construct can insert non-homologously into a random region of the genome where it has no effect on the gene which has been targeted for deletion, and where it can potentially recombine so as to disrupt another gene which was otherwise not intended to be altered. Such non-homologous recombination events can be selected against by modifying the above-mentioned targeting vectors so that they are flanked by negative selectable markers at either end (particularly through the use of the diphtheria toxin gene, thymidine kinase gene, the polypeptide product of which can be selected against in expressing cell lines in an appropriate tissue culture medium well known in the art—e.g., one containing a drug such as ganciclovir. Non-homologous recombination between the resulting targeting vector comprising the negative selectable marker and the genome will usually result in the stable integration of one or both of these negative selectable marker genes and hence cells which have undergone non-homologous recombination can be selected against by growth in the appropriate selective media (e.g., media containing a drug such as ganciclovir). Simultaneous selection for the positive selectable marker and against the negative selectable marker will result in a vast enrichment for clones in which the construct has recombined homologously at the locus of the gene intended to be mutated. The presence of the predicted chromosomal alteration at the targeted gene locus in the resulting stem cell line can be confirmed by means of Southern blot analytical techniques which are well known to those familiar in the art. Alternatively, PCR can be used.

Other methods of making transgenic animals are also generally known. See, for example, Manipulating the Mouse Embryo, (Cold Spring Harbor Laboratory Press, Cold Spring Harbor, N.Y., 1986). Recombinase dependent transgenic organisms can also be generated, e.g., by homologous recombination to insert target sequences, such that tissue specific and/or temporal control of inactivation of a Ryk gene can be controlled by recombinase sequences.

Accordingly, in one aspect, the invention provides a transgenic non-human mammal such as a mouse whose genome comprises a heterozygous or homozygous deletion, inactivation or knock-out of the Ryk gene and methods of making the same. In various embodiments, the mouse has the phenotype Frizzled3.sup.−/−Ryk.sup.+/−. In various embodiments, the mouse contains a corticospinal tract (CST)-specific disruption of the Ryk gene. In various embodiments, the disrupted Ryk gene includes a recombinant Ryk allele, a selectable marker, frt sites flanking the selectable marker, and loxP sites flanking a portion of the allele. The marker may be PGK Neo and the loxP sites may flank exons 3-6 of the allele. Also provided is an isolated cell derived from the transgenic non-human mammal.

B. Isolated Anti-Ryk Antibodies and Related Compositions

In one aspect or embodiment, the present disclosure provides for an isolated anti-Ryk antibody or antibody derivative that: a) specifically binds to a Wnt-binding domain on Ryk or specifically binds to an epitope within a region of the ectodomain of Ryk, e.g., amino-acids 35-211 of mouse Ryk (SEQ ID NO:24) or amino-acids 26-227 of human Ryk (SEQ ID NO:25), the antibody or antibody derivative comprising a light chain variable region comprising the CDR sequence set forth in SEQ ID NO:1 [RANRLVE]; b) specifically binds to the same epitope on a Wnt-binding domain on Ryk or the same epitope within a region of the ectodomain of Ryk, e.g., amino-acids 35-211 of mouse Ryk (SEQ ID NO:24) or amino-acids 26-227 of human Ryk (SEQ ID NO:25), as does a reference antibody or antibody derivative, or cross-competes for specific binding to the Wnt-binding domain on Ryk or the same epitope within a region of the ectodomain of Ryk with a reference antibody or antibody derivative, the reference antibody or antibody derivative comprising a light chain variable region comprising the CDR sequence set forth in SEQ ID NO:1 [RANRLVE]; c) specifically binds to a Wnt-binding domain on Ryk or specifically binds to an epitope within a region of the ectodomain of Ryk, e.g., amino-acids 35-211 of mouse Ryk (SEQ ID NO:24) or amino-acids 26-227 of human Ryk (SEQ ID NO:25), said antibody or antibody derivative comprising a heavy chain variable region comprising the CDR sequence set forth in SEQ ID NO:2 [STGGGGTY], SEQ ID NO:3 [HGDSGDY] or SEQ ID NO:4 [HGDQGDY]; and/or d) specifically binds to the same epitope on a Wnt-binding domain on Ryk or the same epitope within a region of the ectodomain of Ryk, e.g., amino-acids 35-211 of mouse Ryk (SEQ ID NO:24) or amino-acids 26-227 of human Ryk (SEQ ID NO:25), as does a reference antibody or antibody derivative, or cross-competes for specific binding to the Wnt-binding domain on Ryk or the same epitope within a region of the ectodomain of Ryk with a reference antibody or antibody derivative, the reference antibody or antibody derivative comprising a heavy chain variable region comprising the CDR sequence set forth in SEQ ID NO:2 [STGGGGTY], SEQ ID NO:3 [HGDSGDY] or SEQ ID NO:4 [HGDQGDY], provided that the antibody or antibody derivative is not an isolated anti-Ryk antibody or antibody derivative disclosed and/or claimed in WO 2017/172733 A1.

In some embodiments, the present isolated anti-Ryk antibody or antibody derivative binds to an epitope within amino-acids 118-211 or amino-acids 195-202 of mouse Ryk (SEQ ID NO:24). In some embodiments, the present isolated anti-Ryk antibody or antibody derivative binds to an epitope within amino-acids 134-227 or 211-218 of human Ryk (SEQ ID NO:25).

The present isolated anti-Ryk antibody or antibody derivative can comprise any suitable light chain variable region or CDR sequence(s) within the light chain variable region. For example, the present isolated anti-Ryk antibody or antibody derivative can comprise a light chain variable region comprising the CDR sequence set forth in SEQ ID NO:1. In some embodiments, the light chain variable region of the present isolated anti-Ryk antibody or antibody derivative further comprises the CDR sequences set forth in SEQ ID NO:5 [KASQDINSYLS] and/or SEQ ID NO:6 [LQYDEFPLT].

The present isolated anti-Ryk antibody or antibody derivative can comprise any suitable heavy chain variable region or CDR sequence(s) within the heavy chain variable region. For example, the present isolated anti-Ryk antibody or antibody derivative can comprise a heavy chain variable region comprising the CDR sequence set forth in SEQ ID NO:2 [STGGGGTY], SEQ ID NO:3 [HGDSGDY] or SEQ ID NO:4 [HGDQGDY]. In some embodiments, the heavy chain variable region of the present isolated anti-Ryk antibody or antibody derivative comprises the CDR sequences set forth in SEQ ID NO:2 [STGGGGTY], SEQ ID NO:7 [GFTFSSY], and one of SEQ ID NO:3 [HGDSGDY], SEQ ID NO:4 [HGDQGDY], or SEQ ID NO:8 [HGDNGDY].

In some embodiments, the light chain variable region of the present isolated anti-Ryk antibody or antibody derivative comprises the CDR sequences set forth in SEQ ID NO:1 [RANRLVE], SEQ ID NO:5 [KASQDINSYLS] and SEQ ID NO:6 [LQYDEFPLT], and the heavy chain variable region of the present isolated anti-Ryk antibody or antibody derivative comprises the CDR sequences set forth in SEQ ID NO:2 [STGGGGTY], SEQ ID NO:7 [GFTFSSY], and one of SEQ ID NO:3 [HGDSGDY], SEQ ID NO:4 [HGDQGD], or SEQ ID NO:8 [HGDNGDY].

The light chain variable region of the present isolated anti-Ryk antibody or antibody derivative can comprise an amino acid sequence comprising at least about 85% sequence identity to SEQ ID NO:11 [VL1], SEQ ID NO:12 [VL2], or SEQ ID NO:13 [VL3]. For example, the light chain variable region of the present isolated anti-Ryk antibody or antibody derivative can comprise an amino acid sequence comprising at least about 85%, 90%, 91%, 92%, 93%,94%, 95%,96%, 97%,98%, 99%, or 100% sequence identity to SEQ ID NO:11 [VL1], SEQ ID NO:12 [VL2], or SEQ ID NO:13 [VL3]. In some embodiments, the light chain variable region comprises the amino acid sequence set forth in SEQ ID NO:11 [VL1], SEQ ID NO:12 [VL2], or SEQ ID NO:13 [VL3].

The heavy chain variable region of the present isolated anti-Ryk antibody or antibody derivative can comprise an amino acid sequence comprising at least about 85% sequence identity to SEQ ID NO:14 [VH1], SEQ ID NO:15 [VH2], SEQ ID NO:16 [VH3], SEQ ID NO:17 [VH4], or SEQ ID NO:18 [VH5]. For example, the heavy chain variable region of the present isolated anti-Ryk antibody or antibody derivative can comprise an amino acid sequence comprising at least about 85%, 90%, 91%, 92%, 93%,94%, 95%,96%, 97%,98%, 99%, or 100% sequence identity to SEQ ID NO:14 [VH1], SEQ ID NO:15 [VH2], SEQ ID NO:16 [VH3], SEQ ID NO:17 [VH4], or SEQ ID NO:18 [VH5]. In some embodiments, the heavy chain variable region comprises the amino acid sequence set forth in SEQ ID NO:14 [VH1], SEQ ID NO:15 [VH2], SEQ ID NO:16 [VH3], SEQ ID NO:17 [VH4], or SEQ ID NO:18 [VH5].

In some embodiments, the light chain variable region of the present isolated anti-Ryk antibody or antibody derivative comprises the amino acid sequence set forth in SEQ ID NO:11 [VL1], SEQ ID NO:12 [VL2], or SEQ ID NO:13 [VL3], and the heavy chain variable region of the present isolated anti-Ryk antibody or antibody derivative comprises the amino acid sequence set forth in SEQ ID NO:14 [VH1], SEQ ID NO:15 [VH2], SEQ ID NO:16 [VH3], SEQ ID NO:17 [VH4], or SEQ ID NO:18 [VH5].

In another aspect or embodiment, the present disclosure provides for an isolated anti-Ryk antibody or antibody derivative that: a) specifically binds to a Wnt-binding domain on Ryk or specifically binds to an epitope within a region of the ectoclornain of Ryk, e.g., amino-acids 35-211 of mouse Ryk (SEQ ID NO:24) or amino-acids 26-227 of human Ryk (SEQ ID NO:25), the antibody or antibody derivative comprises a light chain variable region comprising an amino acid sequence comprising at least about 85% sequence identity to SEQ ID NO:11 [VL1], SEQ ID NO:12 [VL2], or SEQ ID NO:13 [VL3]; b) specifically binds to the same epitope on a Win-binding domain on Ryk or the same epitope within a region of the ectodomain of Ryk, e.g., amino-acids 35-211 of mouse Ryk (SEQ ID NO:24) or amino-acids 26-227 of human Ryk (SEQ ID NO:25), as does a reference antibody or antibody derivative, or cross-competes for specific binding to the Writ-binding domain on Ryk or the same epitope within a region of the ectodomain of Ryk with a reference antibody or antibody derivative, the reference antibody or antibody derivative comprising a light chain variable region comprising an amino acid sequence comprising at least about 85% sequence identity to SEQ ID NO:11 [VL1], SEQ ID NO:12 [VL2], or SEQ ID NO:13 [VL3]; c) specifically binds to a Wnt-binding domain on Ryk or specifically binds to an epitope within a region of the ectodomain of Ryk, e.g., amino-acids 35-211 of mouse Ryk (SEQ ID NO:24) or amino-acids 26-227 of human Ryk (SEQ ID NO:25), the antibody or antibody derivative comprises a heavy chain variable region that comprises an amino acid sequence comprising at least about 85% sequence identity to SEQ ID NO:14 [VH1], SEQ ID NO:15 [VH2], SEQ ID NO:16 [VH3], SEQ ID NO:17 [VH4], or SEQ ID NO:18 [VH5]; and/or d) specifically binds to the same epitope on a Wnt-binding domain on Ryk or the same epitope within a region of the ectodomain of Ryk, e.g., amino-acids 35-211 of mouse Ryk (SEQ ID NO:24) or amino-acids 26-227 of human Ryk (SEQ ID NO:25), as does a reference antibody or antibody derivative, or cross-competes for specific binding to the Wnt-binding domain on Ryk or the same epitope within a region of the ectodomain of Ryk with a reference antibody or antibody derivative, the reference antibody or antibody derivative comprising a heavy chain variable region that comprises an amino acid sequence comprising at least about 85% sequence identity to SEQ ID NO:14 [VH1], SEQ ID NO:15 [VH2], SEQ ID NO:16 [VH3], SEQ ID NO:17 [VH4], or SEQ ID NO:18 [VH5], provided that the antibody or antibody derivative is not an isolated anti-Ryk antibody or antibody derivative disclosed and/or claimed in WO 2017/172733 A1.

The present isolated anti-Ryk antibody or antibody derivative can comprise any suitable light chain variable region or CDR sequence(s) within the light chain variable region. For example, the light chain variable region of the present isolated anti-Ryk antibody or antibody derivative can comprise the amino acid sequence set forth in SEQ ID NO:11 [VL1], SEQ ID NO:12 [VL2], or SEQ ID NO:13 [VL3].

The present isolated anti-Ryk antibody or antibody derivative can comprise any suitable heavy chain variable region or CDR sequence(s) within the heavy chain variable region. For example, the heavy chain variable region of the present isolated anti-Ryk antibody or antibody derivative can comprise the amino acid sequence set forth in SEQ ID NO:14 [VH1], SEQ ID NO:15 [VH2], SEQ ID NO:16 [VH3], SEQ ID NO:17 [VH4], or SEQ ID NO:18 [VH5].

The light chain variable region of the present isolated anti-Ryk antibody or antibody derivative can comprise the amino acid sequence set forth in SEQ ID NO:11 [VL1], SEQ ID NO:12 [VL2], or SEQ ID NO:13 [VL3], and the heavy chain variable region of the present isolated anti-Ryk antibody or antibody derivative can comprise the amino acid sequence set forth in SEQ ID NO:14 [VH1], SEQ ID NO:15 [VH2], SEQ ID NO:16 [VH3], SEQ ID NO:17 [VH4], or SEQ ID NO:18 [VH5].

In some embodiments, the light chain variable region of the present isolated anti-Ryk antibody or antibody derivative comprises the amino acid sequence set forth in SEQ ID NO: 11 [VL1] and the heavy chain variable region of the present isolated anti-Ryk antibody or antibody derivative comprises the amino acid sequence set forth in SEQ ID NO: 14 [VH1].

In some embodiments, the light chain variable region of the present isolated anti-Ryk antibody or antibody derivative comprises the amino acid sequence set forth in SEQ ID NO: 11 [VL1] and the heavy chain variable region of the present isolated anti-Ryk antibody or antibody derivative comprises the amino acid sequence set forth in SEQ ID NO: 15 [VH2].

In some embodiments, the light chain variable region of the present isolated anti-Ryk antibody or antibody derivative comprises the amino acid sequence set forth in SEQ ID NO: 11 [VL1] and the heavy chain variable region of the present isolated anti-Ryk antibody or antibody derivative comprises the amino acid sequence set forth in SEQ ID NO: 16 [VH3].

In some embodiments, the light chain variable region of the present isolated anti-Ryk antibody or antibody derivative comprises the amino acid sequence set forth in SEQ ID NO: 11 [VL1] and the heavy chain variable region of the present isolated anti-Ryk antibody or antibody derivative comprises the amino acid sequence set forth in SEQ ID NO: 17 [VH4].

In some embodiments, the light chain variable region of the present isolated anti-Ryk antibody or antibody derivative comprises the amino acid sequence set forth in SEQ ID NO: 11 [VL1] and the heavy chain variable region of the present isolated anti-Ryk antibody or antibody derivative comprises the amino acid sequence set forth in SEQ ID NO: 18 [VH5].

In some e embodiments, the light chain variable region of the present isolated anti-Ryk antibody or antibody derivative comprises the amino acid sequence set forth in SEQ ID NO: 12 [VL2] and the heavy chain variable region of the present isolated anti-Ryk antibody or antibody derivative comprises the amino acid sequence set forth in SEQ ID NO: 14 [VH1].

In some embodiments, the light chain variable region of the present isolated anti-Ryk antibody or antibody derivative comprises the amino acid sequence set forth in SEQ ID NO: 12 [VL2] and the heavy chain variable region of the present isolated anti-Ryk antibody or antibody derivative comprises the amino acid sequence set forth in SEQ ID NO:15 [VH2].

In some embodiments, the light chain variable region of the present isolated anti-Ryk antibody or antibody derivative comprises the amino acid sequence set forth in SEQ ID NO: 12 [VL2] and the heavy chain variable region of the present isolated anti-Ryk antibody or antibody derivative comprises the amino acid sequence set forth in SEQ ID NO:16 [VH3].

In some embodiments, the light chain variable region of the present isolated anti-Ryk antibody or antibody derivative comprises the amino acid sequence set forth in SEQ ID NO: 12 [VL2] and the heavy chain variable region of the present isolated anti-Ryk antibody or antibody derivative comprises the amino acid sequence set forth in SEQ ID NO:17 [VH4].

In some embodiments, the light chain variable region of the present isolated anti-Ryk antibody or antibody derivative comprises the amino acid sequence set forth in SEQ ID NO: 12 [VL2] and the heavy chain variable region of the present isolated anti-Ryk antibody or antibody derivative comprises the amino acid sequence set forth in SEQ ID NO:18 [VH5].

In some embodiments, the light chain variable region of the present isolated anti-Ryk antibody or antibody derivative comprises the amino acid sequence set forth in SEQ ID NO:13 [VL3] and the heavy chain variable region of the present isolated anti-Ryk antibody or antibody derivative comprises the amino acid sequence set forth in SEQ ID NO:14 [VH1].

In some embodiments, the light chain variable region of the present isolated anti-Ryk antibody or antibody derivative comprises the amino acid sequence set forth in SEQ ID NO: 13 [VL3] and the heavy chain variable region of the present isolated anti-Ryk antibody or antibody derivative comprises the amino acid sequence set forth in SEQ ID NO:15 [VH2].

In some embodiments, the light chain variable region of the present isolated anti-Ryk antibody or antibody derivative comprises the amino acid sequence set forth in SEQ ID NO: 13 [VL3] and the heavy chain variable region of the present isolated anti-Ryk antibody or antibody derivative comprises the amino acid sequence set forth in SEQ ID NO:16 [VH3].

In some embodiments, the light chain variable region of the present isolated anti-Ryk antibody or antibody derivative comprises the amino acid sequence set forth in SEQ ID NO: 13 [VL3] and the heavy chain variable region of the present isolated anti-Ryk antibody or antibody derivative comprises the amino acid sequence set forth in SEQ ID NO:17 [VH4].

In some embodiments, the light chain variable region of the present isolated anti-Ryk antibody or antibody derivative comprises the amino acid sequence set forth in SEQ ID NO: 13 [VL3] and the heavy chain variable region of the present isolated anti-Ryk antibody or antibody derivative comprises the amino acid sequence set forth in SEQ ID NO:18 [VH5].

The present isolated anti-Ryk antibody or antibody derivative can be in any suitable form. For example, the present isolated anti-Ryk antibody or antibody derivative can be a humanized antibody, e.g., a humanized monoclonal antibody. In another example, the present isolated anti-Ryk antibody or antibody derivative can be a polyclonal antibody, a monoclonal antibody, an antibody fragment, a single chain antibody, a single domain antibody, e.g., sdAb, sdFv, or nanobody, an intrabody, a peptibody, a chimeric antibody, a fully human antibody, a humanized antibody, a heteroconjugate antibody, a multispecific antibody, e.g., a bispecific antibody, a diabody, a triabody, a tetrabody, a tandem di-scFv, or a tandem tri-scFv. The antibody fragment can be in any suitable form. For example, the antibody fragment can be an antigen binding (Fab) fragment, a F(ab′)₂ fragment, a Fab′ fragment, a Fv fragment, a recombinant IgG (rIgG) fragment, a single chain antibody fragment, e.g., a single chain variable fragment (scFv), or a single domain antibody fragment.

The present isolated anti-Ryk antibody or antibody derivative can inhibit or reduce Ryk binding to Wnt, and/or inhibit or reduce the planar cell polarity signaling pathway, to or by any suitable degree. For example, the present isolated anti-Ryk antibody or antibody derivative can inhibit or reduce Ryk binding to Wnt by at least 10%, 20%, 30%, 40%, 50%, 60%, 70%, 80%, 90%, 91%, 92%, 93%,94%, 95%,96%, 97%,98%, 99%, or 100%.

The present isolated anti-Ryk antibody or antibody derivative can specifically bind to an epitope within amino acid residues 90-183 of Ryk. For example, the present isolated anti-Ryk antibody or antibody derivative can bind to an epitope within or comprising the amino acid sequence set forth in SEQ ID NO:19 [SRTIYDPV] or an epitope within or comprising an amino acid sequence having at least about 80% sequence identity to the amino acid sequence set forth in SEQ ID NO:19 [SRTIYDPV]. In some embodiments, the present isolated anti-Ryk antibody or antibody derivative can bind to an epitope within or comprising an amino acid sequence having at least about 80%, 85%, 90%, 91%, 92%, 93%,94%, 95%,96%, 97%,98%, or 99% sequence identity to the amino acid sequence set forth in SEQ ID NO:19 [SRTIYDPV]. In some embodiments, the present isolated anti-Ryk antibody or antibody derivative binds to an epitope within or comprising the amino acid sequence set forth in SEQ ID NO:20 [ARTIYDPV], SEQ ID NO:21 [PRTIYDPV] or SEQ ID NO:22 [SRTLYDPV]. In some embodiments, the present isolated anti-Ryk antibody or antibody derivative binds to an epitope within or comprising the amino acid sequence set forth in SEQ ID NO:23 [SRXIYDPV], X being a natural amino acid that is not T.

The present isolated anti-Ryk antibody or antibody derivative can have lower immunogenicity than Ab5.5 disclosed and/or claimed in WO 2017/172733 A1 in a human. The present isolated anti-Ryk antibody or antibody derivative can have lower immunogenicity than Ab5.5 disclosed and/or claimed in WO 2017/172733 A1 in a human by any suitable degree. For example, the present isolated anti-Ryk antibody or antibody derivative can have a DRB1 risk score that is at least about 30% or 40% lower than the DRB1 risk score of Ab5.5 disclosed and/or claimed in WO 2017/172733 A1. In some embodiments, the present isolated anti-Ryk antibody or antibody derivative has a DRB1 risk score that is at least about 40%, 50%, 60%, 70%, 80%, 90%, or 95% lower than the DRB1 risk score of Ab5.5 disclosed and/or claimed in WO 2017/172733 A1.

The present isolated anti-Ryk antibody or antibody derivative can have any suitable DRB1 risk score. For example, the present isolated anti-Ryk antibody or antibody derivative can have a DRB1 risk score ranging from about 500 to about 700. In some embodiments, the present isolated anti-Ryk antibody or antibody derivative has a DRB1 risk score of about 500, 550, 600, 650, 700, or any subrange thereof.

The present isolated anti-Ryk antibody or antibody derivative can have any suitable binding affinity or strength to a Ryk polypeptide. For example, the present isolated anti-Ryk antibody or antibody derivative can have a KD value for binding to a Ryk polypeptide ranging from about 0.01 pM to about 500 pM, e.g., a KD value at about 0.01 pM, 0.1 pM, 1 pM, 10 pM, pM, 30 pM, 40 pM, 50 pM, 60 pM, 70 pM, 80 pM, 90 pM, 100 pM, 200 pM, 300 pM, 400 pM, 500 pM, or any subrange thereof.

In still another aspect or embodiment, the present disclosure provides for an immunoconjugate comprising the above isolated antibody or antibody derivative, linked to a detecting and/or therapeutic agent. The present immunoconjugate can comprise any suitable detecting or therapeutic agent. For example, the detecting or therapeutic agent can be a cytotoxin or a radioactive isotope.

In yet another aspect or embodiment, the present disclosure provides for a bispecific molecule comprising the above isolated antibody or antibody derivative, linked to a second functional moiety having a different binding specificity than the present isolated antibody or antibody derivative.

In yet another aspect or embodiment, the present disclosure provides for a pharmaceutical composition comprising an effective amount of the above antibody or antibody derivative, the above immunoconjugate, or the above bispecific molecule, and a pharmaceutically acceptable carrier or excipient.

In yet another aspect or embodiment, the present disclosure provides for a nucleic acid encoding the above isolated antibody or antibody derivative of or the above bispecific molecule.

In yet another aspect or embodiment, the present disclosure provides for a vector comprising the above nucleic acid. The vector can be in any suitable form. For example, the vector can be an expression vector.

In some embodiments, recombinant nucleic acids encoding anti-Ryk antibodies are particularly useful for expression in a host cell that in effect serves as a factory for the anti-Ryk antibodies. In various embodiments, nucleic acids are isolated when purified away from other cellular components or other contaminants (e.g., other nucleic acids or proteins present in the cell) by standard techniques including, including alkaline/SDS treatment, CsCl banding, column chromatography, agarose gel electrophoresis and others well-known in the art. See e.g., F. Ausubel, et al., ed. (1987) Current Protocols in Molecular Biology, Greene Publishing and Wiley Interscience, New York. In various embodiments, a nucleic acid is, for example, DNA or RNA and may or may not contain intronic sequences. In a preferred embodiment, the nucleic acid is a cDNA molecule. In various embodiments, a recombinant nucleic acid provides a recombinant gene encoding the anti-Ryk antibody that exists autonomously from a host cell genome or as part of the host cell genome.

In some embodiments, a recombinant gene contains nucleic acids encoding a protein along with regulatory elements for protein expression. Generally, the regulatory elements that are present in a recombinant gene include a transcriptional promoter, a ribosome binding site, a terminator, and an optionally present operator. A promoter is defined as a DNA sequence that directs RNA polymerase to bind to DNA and initiate RNA synthesis. Antibody associated introns may also be present. The degeneracy of the genetic code is such that, for all but two amino acids, more than a single codon encodes a particular amino acid. This allows for the construction of synthetic DNA that encodes a protein where the nucleotide sequence of the synthetic DNA differs significantly from the nucleotide sequences disclosed herein, but still encodes such a protein. Such synthetic DNAs are intended to be within the scope of the present invention.

In yet another aspect or embodiment, the present disclosure provides for a host cell comprising the above vector. The host cell can be in any suitable form. For example, the host cell can be a mammalian host cell, e.g., a human host cell.

In yet another aspect or embodiment, the present disclosure provides for a transgenic non-human animal, e.g., a transgenic mouse, comprising the above host cell, wherein the non-human animal or mouse expresses a polypeptide encoded by the nucleic acid.

Antibodies of the invention can be administered by any suitable means, including parenteral, subcutaneous, intraperitoneal, intrapulmonary, intranasal, and, if desired for local treatment, intralesional administration. Parenteral infusions include intramuscular, intravenous, intraarterial, intraperitoneal, and subcutaneous administration. In addition, antibodies can be administered by pulse infusion, particularly with declining doses of the antibody. Dosing can be by any suitable route, e.g., by injections, such as intravenous or subcutaneous injections, depending in part on whether the administration is brief or chronic.

In some embodiments, the term “antibody” is used in its broadest sense to include polyclonal and monoclonal antibodies, as well as antigen binding fragments of such antibodies. Antibodies are characterized, in part, in that they specifically bind to an antigen, particularly to one or more epitopes of an antigen. In some embodiments, the term “binds specifically” or “specific binding activity” or the like, when used in reference to an antibody, means that an interaction of the antibody and a particular epitope has a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or less. As such, Fab, F(ab′).sub.2, Fd and Fv fragments of an antibody that retain specific binding activity are included within the definition of an antibody.

In some embodiments, the term “antibody” as used herein includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246:1275-1281, 1989, which is incorporated herein by reference). These and other methods of making, for example, chimeric, humanized, CDR-grafted, single chain, and bifunctional antibodies are well known (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1999); Hilyard et al., Protein Engineering: A practical approach (IRL Press 1992); Borrabeck, Antibody Engineering, 2d ed. (Oxford University Press 1995); each of which is incorporated herein by reference). In addition, modified or derivatized antibodies, or antigen binding fragments of antibodies, such as pegylated (polyethylene glycol modified) antibodies, can be useful for the present methods.

Antibodies can be tested for anti-target polypeptide activity using a variety of methods well-known in the art. Various techniques may be used for screening to identify antibodies having the desired specificity, including various immunoassays, such as enzyme-linked immunosorbent assays (ELISAs), including direct and ligand-capture ELISAs, radioimmunoassays (RIAs), immunoblotting, and fluorescent activated cell sorting (FACS). Numerous protocols for competitive binding or immunoradiometric assays, using either polyclonal or monoclonal antibodies with established specificities, are well known in the art. Such immunoassays typically involve the measurement of complex formation between the target polypeptide and a specific antibody. A two-site, monoclonal-based immunoassay utilizing monoclonal antibodies reactive to two non-interfering epitopes on the target polypeptide is preferred, but other assays, such as a competitive binding assay, may also be employed. See, e.g., Maddox et al, 1983, J. Exp. Med. 158:1211.

The location of the binding target of an antibody used in the invention can be taken into consideration in preparation and administration of the antibody. When the binding target is an intracellular molecule, certain embodiments of the invention provide for the antibody or antigen-binding fragment thereof to be introduced into the cell where the binding target is located. In one embodiment, an antibody of the invention can be expressed intracellularly as an intrabody. The term “intrabody,” as used herein, refers to an antibody or antigen-binding portion thereof that is expressed intracellularly and that is capable of selectively binding to a target molecule, as described in Marasco, Gene Therapy 4:11-15, 1997; Kontermann, Methods 34:163-170, 2004; U.S. Pat. Nos. 6,004,940 and 6,329,173; U.S. Patent Application Publication No. 2003/0104402, and PCT Publication No. WO 03/077945. Intracellular expression of an intrabody is effected by introducing a nucleic acid encoding the desired antibody or antigen-binding portion thereof (lacking the wild-type leader sequence and secretory signals normally associated with the gene encoding that antibody or antigen-binding fragment) into a target cell. Any standard method of introducing nucleic acids into a cell may be used, including, but not limited to, microinjection, ballistic injection, electroporation, calcium phosphate precipitation, liposomes, and transfection with retroviral, adenoviral, adeno-associated viral and vaccinia vectors carrying the nucleic acid of interest.

In another embodiment, internalizing antibodies are provided. Antibodies can possess certain characteristics that enhance delivery of antibodies into cells, or can be modified to possess such characteristics. Techniques for achieving this are known in the art. For example, cationization of an antibody is known to facilitate its uptake into cells (see, e.g., U.S. Pat. No. 6,703,019). Lipofections or liposomes can also be used to deliver the antibody into cells. Where antibody fragments are used, the smallest inhibitory fragment that specifically binds to the binding domain of the target protein is generally advantageous. For example, based upon the variable-region sequences of an antibody, peptide molecules can be designed that retain the ability to bind the target protein sequence. Such peptides can be synthesized chemically and/or produced by recombinant DNA technology (see, e.g., Marasco et al., Proc. Natl. Acad. Sci. U.S.A. 90:7889-7893, 1993).

Entry of modulator polypeptides into target cells can be enhanced by methods known in the art. For example, certain sequences, such as those derived from HIV Tat or the Antennapedia homeodomain protein are able to direct efficient uptake of heterologous proteins across cell membranes (see, e.g., Chen et al., Proc. Natl. Acad. Sci. U.S.A. 96:4325-4329, 1999).

When the binding target is located in the brain, certain embodiments of the invention provide for the antibody or antigen-binding fragment thereof to traverse the blood-brain barrier. Certain neurological/neurodegenerative diseases are associated with an increase in permeability of the blood-brain barrier, such that the antibody or antigen-binding fragment can be readily introduced to the brain. When the blood-brain barrier remains intact, several art-known approaches exist for transporting molecules across it, including, but not limited to, physical methods, lipid-based methods, and receptor and channel-based methods.

Physical methods of transporting the antibody or antigen-binding fragment across the blood-brain barrier include, but are not limited to, circumventing the blood-brain barrier entirely, or by creating openings in the blood-brain barrier. Circumvention methods include, but are not limited to, direct injection into the brain (see, e.g., Papanastassiou et al., Gene Therapy 9:398-406, 2002), interstitial infusion/convection-enhanced delivery (see, e.g., Bobo et al., Proc. Natl. Acad. Sci. U.S.A. 91:2076-2080, 1994), and implanting a delivery device in the brain (see, e.g., Gill et al., Nature Med. 9:589-595, 2003; and Gliadel Wafers™, Guildford Pharmaceutical). Methods of creating openings in the barrier include, but are not limited to, ultrasound (see, e.g., U.S. Pub. No. 2002/0038086), osmotic pressure (e.g., by administration of hypertonic mannitol (Neuwelt, E. A., Implication of the Blood-Brain Barrier and its Manipulation, Volumes 1 and 2, Plenum Press, N.Y., 1989)), permeabilization by, e.g., bradykinin or permeabilizer A-7 (see, e.g., U.S. Pat. Nos. 5,112,596, 5,268,164, 5,506,206, and 5,686,416), and transfection of neurons that straddle the blood-brain barrier with vectors containing genes encoding the antibody or antigen-binding fragment (see, e.g., U.S. Pub. No. 2003/0083299).

Lipid-based methods of transporting the antibody or antigen-binding fragment across the blood-brain barrier include, but are not limited to, encapsulating the antibody or antigen-binding fragment in liposomes that are coupled to antibody binding fragments that bind to receptors on the vascular endothelium of the blood-brain barrier (see, e.g., U.S. Pub. No. 2002/0025313), and coating the antibody or antigen-binding fragment in low-density lipoprotein particles (see, e.g., U.S. Pub. No. 2004/0204354) or apolipoprotein E (see, e.g., U.S. Pub. No. 2004/0131692).

Receptor and channel-based methods of transporting the antibody or antigen-binding fragment across the blood-brain barrier include, but are not limited to, using glucocorticoid blockers to increase permeability of the blood-brain barrier (see, e.g., U.S. Pub. Nos. 2002/0065259, 2003/0162695, and 2005/0124533); activating potassium channels (see, e.g., U.S. Pub. No. 2005/0089473), inhibiting ABC drug transporters (see, e.g., U.S. Pub. No. 2003/0073713); coating antibodies with a transferrin and modulating activity of the one or more transferrin receptors (see, e.g., U.S. Pub. No. 2003/0129186), and cationizing the antibodies (see, e.g., U.S. Pat. No. 5,004,697).

Antibody compositions used in the methods of the invention are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibodies of the invention present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody (when used alone or in combination with other agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g., 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg, or 10 mg/kg (or any combination thereof) may be administered to the patient. Such doses may be administered intermittently, e.g., every week or every three weeks (e.g., such that the patient receives from about two to about twenty, or, e.g., about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

In some embodiments, different antibody regions are illustrated by reference to IgG, which contains four amino acid chains—two longer length heavy chains and two shorter light chains that are inter-connected by disulfide bonds. The heavy and light chains each contain a constant region and a variable region. A heavy chain is comprised of a heavy chain variable region and a heavy chain constant region. A light chain is comprised of a light chain variable region and a light chain constant region. In various embodiments, there are three hypervariable regions within the variable regions that are responsible for antigen specificity. In various embodiments, the hypervariable regions are referred to as complementarity determining regions (CDR) and are interposed between more conserved flanking regions referred to as framework regions (FW). In various embodiments, the variable regions of the heavy and light chains contain a binding domain that interacts with an antigen.

C. Uses of the Anti-Ryk Antibodies and Related Compositions

In yet another aspect or embodiment, the present disclosure provides for a method of interfering with interaction of Wnt and Ryk comprising contacting a sample comprising Wnt and Ryk with the above isolated antibody or antibody derivative, the above immunoconjugate, or the above bispecific molecule, thereby interfering with the interaction of Wnt and Ryk.

In yet another aspect or embodiment, the present disclosure provides for a method for inhibiting degeneration of a neuron, the method comprising contacting the neuron with the above isolated antibody or antibody derivative, the above immunoconjugate, the above bispecific molecule, the above pharmaceutical composition, the above nucleic acid, the above vector, or the above host cell, thereby inhibiting degeneration of the neuron.

The present methods can be used for inhibiting degeneration of a neuron in any suitable manner. For example, degeneration of an axon of the neuron can be inhibited. In another example, degeneration of a cell body of the neuron can be inhibited. The present methods can be used for inhibiting degeneration of any suitable types of axon. For example, the present methods can be used for inhibiting degeneration of a spinal cord commissural axon, an upper motor neuron axon or a central nervous system axon.

The present methods can be used for inhibiting degeneration of any suitable types of neurons. For example, the present methods can be used for inhibiting degeneration of a damaged spinal cord neuron, a sensory neuron, a motor neuron, a cerebellar granule neuron, a dorsal root ganglion neuron, a cortical neuron, a sympathetic neuron, or a hippocampal neuron. In another example, the present methods can be used for inhibiting degeneration of a neuron that forms part of a nerve graft or a nerve transplant. The nerve graft or the nerve transplant can be or form part of an organism.

The present methods can be used for inhibiting degeneration of a neuron in any suitable manner. For example, the neuron can be contacted with the above isolated antibody or antibody derivative, the above immunoconjugate, the above bispecific molecule, the above pharmaceutical composition, the above nucleic acid, the above vector or the above host cell ex vivo or in vitro.

The present methods can be used for inhibiting degeneration of a neuron in any suitable organism. For example, the present methods can be used for inhibiting degeneration of a neuron in a mammal. In another example, the present methods can be used for inhibiting degeneration of a neuron in a human.

In yet another aspect or embodiment, the present disclosure provides for a method of preventing or treating a neurological disease, disorder or injury in a subject having or being at risk of developing the neurological disease, disorder or injury comprising administering to the subject an effective amount of the above isolated antibody or antibody derivative, the above immunoconjugate, the above bispecific molecule, the above pharmaceutical composition, the above nucleic acid, the above vector, or the above host cell, thereby treating the neurological disease, disorder or injury in the subject.

The present methods can be used for preventing or treating any suitable neurological disease, disorder or injury in a subject. For example, the present methods can be used for preventing or treating a neurodegenerative disease or disorder, e.g., amyotrophic lateral sclerosis, Alzheimer's disease or Parkinson's disease. In another example, the present methods can be used for preventing or treating a spinal cord injury, a traumatic brain injury, or a peripheral nerve injury.

In yet another aspect or embodiment, the present disclosure provides for a method for modulating the directional growth of a neuron comprising contacting the neuron with the above isolated antibody or antibody derivative, the above immunoconjugate, the above bispecific molecule, the above pharmaceutical composition, the above nucleic acid, the above vector, or the above host cell, thereby modulating the directional growth of the neuron.

The present methods can be used for modulating the directional growth of any suitable neuron. For example, the present methods can be used for modulating the directional growth of a spinal cord commissural axon, an upper motor neuron axon, a central nervous system axon, a peripheral nervous system axon, a damaged spinal cord neuron, a sensory neuron, or a motor neuron. In some embodiments, the directional growth facilitates regeneration of the neuron.

In yet another aspect or embodiment, the present disclosure provides for an use of an effective amount of the above isolated antibody or antibody derivative, the above immunoconjugate, the above bispecific molecule, the above nucleic acid, the above vector, or the above host cell for manufacturing a medicament for treating or preventing a neurological disease, disorder or injury in a subject having or being at risk of developing the neurological disease, disorder or injury. The above isolated antibody or antibody derivative, the above immunoconjugate, the above bispecific molecule, the above nucleic acid, the above vector, or the above host cell can be used for manufacturing a medicament for treating or preventing any suitable neurological disease, disorder or injury. For example, the neurological disease or disorder can be a neurodegenerative disease or disorder.

As used herein, the term “neuron” include a neuron and a portion or portions thereof (e.g., the neuron cell body, an axon, or a dendrite). The term “neuron” as used herein denotes nervous system cells that include a central cell body or soma, and two types of extensions or projections: dendrites, by which, in general, the majority of neuronal signals are conveyed to the cell body, and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal column). Certain specific examples of neuron types that may be subject to treatment or methods according to the invention include cerebellar granule neurons, dorsal root ganglion neurons, and cortical neurons.

The term “neuronal degeneration” is used broadly and refers to any pathological changes in neuronal cells, including, without limitation, death or loss of neuronal cells, any changes that precede cell death, and any reduction or loss of an activity or a function of the neuronal cells. The pathological changes may be spontaneous or may be induced by any event and include, for example, pathological changes associated with apoptosis. The neurons may be any neurons, including without limitation sensory, sympathetic, parasympathetic, or enteric, e.g., dorsal root ganglia neurons, motor neurons, and central neurons, e.g., neurons from the spinal cord. Neuronal degeneration or cell loss is a characteristic of a variety of neurological diseases or disorders, e.g., neurodegenerative diseases or disorders. In some embodiments, the neuron is a sensory neuron. In some embodiments, the neuron is a motor neuron. In some embodiments, the neuron is a damaged spinal cord neuron.

In some embodiments, degeneration occurs in a portion of the neuron such as the neuron cell body, an axon, or a dendrite. Accordingly, the degeneration can be inhibited in the degenerated portion or portions of the neuron. In some embodiments, the degeneration of an axon of the neuron is inhibited. In some embodiments, the degeneration of a cell body of the neuron is inhibited. The axon can be an axon of any neuron. For example, in some embodiments, the axon is a spinal cord commissural axon, or an upper motor neuron axon, or a central nervous system axon.

In some embodiments, axon degeneration is a common feature in many neurological and neurodegenerative diseases/disorders and in traumatic injuries. Studies indicate that it can occur independent of and before the death of neuronal cell bodies. However, the molecular and cellular mechanisms underlying axonal degeneration and protection are still unclear. Elucidating the degeneration pathways that are activated or the protection pathways that are inactivated during axon pathology will help develop specific therapeutic agents that preserve axon integrity and enhance regeneration.

During the development of the nervous system, axons respond to extracellular signals that promote the growth as well as those that inhibit their growth. Some extracellular cues attract axons to grow towards higher concentration and others repel axon away from higher concentration. The signaling pathways that regulate these opposite axon responses have profound effect on the extension and removal of axons, although their functions in mature axons have not been well characterized. Studies suggest that axon guidance molecules may play a role in neurological/neurodegenerative disorders, such as amyotrophic lateral sclerosis (ALS).

In some embodiments, the present invention provides methods and compositions for modulating growth of a nerve cell by contacting the neuron with an agent, thereby inhibiting degeneration of a neuron. In various embodiments, the agent may be an anti-Ryk monoclonal antibody or antibody fragment that specifically binds to a binding domain of Wnt affecting a Wnt signaling pathway. These methods and compositions can be used in a wide variety of therapeutic contexts where nerve growth and regeneration would be beneficial. For example, an anti-Ryk antibody or antibody fragment affecting a Wnt signaling pathway can be used to stimulate axonal growth of a damaged neuron along the A-P axis of a patient with SCI. Because it has also been observed that the Wnts are expressed in the several regions in the brain and the components of the Wnt signaling pathways are also present in axons of other central nervous system neurons, it is possible that the anti-Ryk antibody or antibody fragments described herein can be used to modulate growth and directional guidance of axons in the central nervous system.

In some embodiments, the methods as described herein result in at least a 10% decrease (e.g., at least 15%, 20%, 25%, 30%, 35%, 40%, 45%, 50%, 55%, 60%, 65%, 70%, 75%, 80%, 85%, 90%, or even 100% decrease) in the degeneration of a population of neurons or in the degeneration of axons or cell bodies or dendrites of a neuron in a population of neurons as compared to a control population of neurons. In some embodiments, the methods as described herein result at least a 10% decrease (e.g., at least 15%, 20%, 25%, 30%, 35%, 40%, 45%, 50%, 55%, 60%, 65%, 70%, 75%, 80%, 85%, 90%, 95%, or even 100% decrease) in the number of neurons (or neuron bodies, axons, or dendrites thereof) that degenerate in a subject compared to the number of neurons (or neuron bodies, axons, or dendrites thereof) that degenerate in a subject that is not administered the one or more of the agents described herein. In some embodiments, the methods as described herein result in at least a 10% decrease (e.g., at least 15%, 20%, 25%, 30%, 35%, 40%, 45%, 50%, 55%, 60%, 65%, 70%, 75%, 80%, 85%, 90%, or even 100% decrease) in one or more (e.g., 1, 2, 3, 4, 5, 6, 7, 8, or 9) symptoms of a neurological/neurodegenerative disease or disorder and/or condition. In some embodiments, the methods as described herein result in at least a 10% decrease (e.g., at least 15%, 20%, 25%, 30%, 35%, 40%, 45%, 50%, 55%, 60%, 65%, 70%, 75%, 80%, 85%, 90%, 95%, or even 100% decrease) in the likelihood of developing a neurological/neurodegenerative disease or disorder and/or condition.

The methods of inhibiting neuron degeneration include in vitro, in vivo, and/or ex vivo methods. In some embodiments, the methods are practiced in vivo, i.e., the agent inhibiting neuron degeneration is administered to a subject. In some embodiments, the methods are practiced ex vivo, i.e., neurons to be treated form part of a nerve graft or a nerve transplant in a subject. In some embodiments, the methods are practiced in vitro.

In some embodiments, the methods of inhibiting neuron degeneration can be used to inhibit or prevent neuron degeneration in patients newly diagnosed as having a neurological/neurodegenerative disease or disorder or at risk of developing a new neurological/neurodegenerative disease or disorder. On the other hand, the methods of inhibiting neuron degeneration can also be used to inhibit or prevent further neuron degeneration in patients who are already suffering from, or have symptoms of, a neurological/neurodegenerative disease or disorder. Preventing neuron degeneration includes decreasing or inhibiting neuron degeneration, which may be characterized by complete or partial inhibition of neuron degeneration. This can be assessed, for example, by analysis of neurological function.

In some embodiments, the anti-Ryk antibodies or antibody fragments described herein can be used in methods for inhibiting neuron (e.g., axon) degeneration. These antibodies or antibody fragments are, therefore, useful in the therapy of, for example, (i) disorders of the nervous system (e.g., neurological/neurodegenerative diseases or disorders), (ii) conditions of the nervous system that are secondary to a disease, condition, or therapy having a primary effect outside of the nervous system, (iii) injuries to the nervous system caused by physical, mechanical, or chemical trauma, (iv) pain, (v) ocular-related neurodegeneration, (vi) memory loss, and (vii) psychiatric disorders. Non-limiting examples of some of these diseases, conditions, and injuries are provided below.

Examples of neurological/neurodegenerative diseases and conditions that can be prevented or treated according to the invention include amyotrophic lateral sclerosis (ALS), trigeminal neuralgia, glossopharyngeal neuralgia, Bell's Palsy, myasthenia gravis, muscular dystrophy, progressive muscular atrophy, primary lateral sclerosis (PLS), pseudobulbar palsy, progressive bulbar palsy, spinal muscular atrophy, progressive bulbar palsy, inherited muscular atrophy, invertebrate disk syndromes (e.g., herniated, ruptured, and prolapsed disk syndromes), cervical spondylosis, plexus disorders, thoracic outlet destruction syndromes, peripheral neuropathies, prophyria, mild cognitive impairment, Alzheimer's disease, Huntington's disease, Parkinson's disease, Parkinson's-plus diseases (e.g., multiple system atrophy, progressive supranuclear palsy, and corticobasal degeneration), dementia with Lewy bodies, frontotemporal dementia, demyelinating diseases (e.g., Guillain-Barre syndrome and multiple sclerosis), Charcot-Marie-Tooth disease (CMT; also known as Hereditary Motor and Sensory Neuropathy (HMSN), Hereditary Sensorimotor Neuropathy (HSMN), and Peroneal Muscular Atrophy), prion disease (e.g., Creutzfeldt-Jakob disease, Gerstmann-Straussler-Scheinker syndrome (GSS), fatal familial insomnia (FFI), and bovine spongiform encephalopathy (BSE, commonly known as mad cow disease)), Pick's disease, epilepsy, and AIDS demential complex (also known as HIV dementia, HIV encephalopathy, and HIV-associated dementia).

In some embodiments, the methods of the invention can also be used in the prevention and treatment of ocular-related neurodegeneration and related diseases and conditions, such as glaucoma, lattice dystrophy, retinitis pigmentosa, age-related macular degeneration (AMD), photoreceptor degeneration associated with wet or dry AMD, other retinal degeneration, optic nerve drusen, optic neuropathy, and optic neuritis. Non-limiting examples of different types of glaucoma that can be prevented or treated according to the invention include primary glaucoma (also known as primary open-angle glaucoma, chronic open-angle glaucoma, chronic simple glaucoma, and glaucoma simplex), low-tension glaucoma, primary angle-closure glaucoma (also known as primary closed-angle glaucoma, narrow-angle glaucoma, pupil-block glaucoma, and acute congestive glaucoma), acute angle-closure glaucoma, chronic angle-closure glaucoma, intermittent angle-closure glaucoma, chronic open-angle closure glaucoma, pigmentary glaucoma, exfoliation glaucoma (also known as pseudoexfoliative glaucoma or glaucoma capsulare), developmental glaucoma (e.g., primary congenital glaucoma and infantile glaucoma), secondary glaucoma (e.g., inflammatory glaucoma (e.g., uveitis and Fuchs heterochromic iridocyclitis)), phacogenic glaucoma (e.g., angle-closure glaucoma with mature cataract, phacoanaphylactic glaucoma secondary to rupture of lens capsule, phacolytic glaucoma due to phacotoxic meshwork blockage, and subluxation of lens), glaucoma secondary to intraocular hemorrhage (e.g., hyphema and hemolytic glaucoma, also known as erythroclastic glaucoma), traumatic glaucoma (e.g., angle recession glaucoma, traumatic recession on anterior chamber angle, postsurgical glaucoma, aphakic pupillary block, and ciliary block glaucoma), neovascular glaucoma, drug-induced glaucoma (e.g., corticosteroid induced glaucoma and alpha-chymotrypsin glaucoma), toxic glaucoma, and glaucoma associated with intraocular tumors, retinal detachments, severe chemical burns of the eye, and iris atrophy.

Certain diseases and conditions having primary effects outside of the nervous system can lead to damage to the nervous system, which can be treated according to the methods of the present invention. Examples of such conditions include peripheral neuropathy and neuralgia caused by, for example, diabetes, cancer, AIDS, hepatitis, kidney dysfunction, Colorado tick fever, diphtheria, HIV infection, leprosy, lyme disease, polyarteritis nodosa, rheumatoid arthritis, sarcoidosis, Sjogren syndrome, syphilis, systemic lupus erythematosus, and amyloidosis.

In addition, the methods of the invention can be used in the treatment of nerve damage, such as peripheral neuropathy, which is caused by exposure to toxic compounds, including heavy metals (e.g., lead, arsenic, and mercury) and industrial solvents, as well as drugs including chemotherapeutic agents (e.g., vincristine and cisplatin), dapsone, HIV medications (e.g., Zidovudine, Didanosine, Stavudine, Zalcitabine, Ritonavir, and Amprenavir), cholesterol lowering drugs (e.g., Lovastatin, Indapamid, and Gemfibrozil), heart or blood pressure medications (e.g., Amiodarone, Hydralazine, Perhexiline), and Metronidazole.

The methods of the invention can also be used to treat injury to the nervous system caused by physical, mechanical, or chemical trauma. Thus, the methods can be used in the treatment of peripheral nerve damage caused by physical injury (associated with, e.g., burns, wounds, surgery, and accidents), ischemia, prolonged exposure to cold temperature (e.g., frost-bite), as well as damage to the central nervous system due to, e.g., stroke or intracranial hemorrhage (such as cerebral hemorrhage).

Further, the methods of the invention can be used in the prevention or treatment of memory loss such as, for example, age-related memory loss. Types of memory that can be affected by loss, and thus treated according to the invention, include episodic memory, semantic memory, short-term memory, and long-term memory. Examples of diseases and conditions associated with memory loss, which can be treated according to the present invention, include mild cognitive impairment, Alzheimer's disease, Parkinson's disease, Huntington's disease, chemotherapy, stress, stroke, and traumatic brain injury (e.g., concussion).

Further, the methods of the invention can be used in the prevention or treatment of neuropathic pain. The present methods can be used to prevent or treat any suitable types of neuropathic pain. For example, the present methods can be used to prevent or treat neuropathic pain that is caused by a lesion or disease of the somatosensory system. In another example, the present methods can be used to prevent or treat peripheral neuropathic pain, central neuropathic pain, or mixed (peripheral and central) neuropathic pain. The isolated antibody or antibody derivative, the immunoconjugate, the bispecific molecule, the pharmaceutical composition, the nucleic acid sequence, the vector, or the host cell can be administered to the subject via any suitable route. For example, the isolated antibody or antibody derivative, the immunoconjugate, the bispecific molecule, the pharmaceutical composition, the nucleic acid sequence, the vector, or the host cell can be administered to the subject via intrathecal administration or infusion.

In some embodiments, neuropathic pain is pain caused by a lesion or disease of the somatosensory system and affects an estimated 7-10% of the general population worldwide^{1,2}. Wnt signaling can be increased in rodent models of neuropathic pain³, and blocking Wnt signaling is currently considered to be a potential therapeutic strategy for neuropathic pain⁴. One strategy for treating neuropathic pain that is supported by multiple in vivo studies is to target the Wnt co-receptor Ryk.

In one example, in rats, chronic constriction injury of the sciatic nerve induces a rapid increase of Ryk, Wnt3a, and Wnt5a in the injured sensory neurons⁵. Intrathecal infusion of an anti-Ryk function blocking antibody after sciatic nerve injury significantly reduced neuropathic pain determined by assessing mechanical allodynia and thermal hyperalgesia⁵.

In another example, in rats, spinal nerve ligation of the L5 spinal nerve in rats resulted in increases in Ryk and Wnt1 mRNA and protein levels in the dorsal root ganglion neurons after the injury⁶. Intrathecal infusion of an anti-Ryk antibody after spinal nerve ligation significantly reduced neuropathic pain assessed by mechanical allodynia but did not impact thermal hyperalgesia in this model⁶.

In still another example, in mice, Wnt5a levels are increased in the spinal cord in models of neuropathic, inflammatory, and cancer pain (spared nerve injury, Complete Freud's Adjuvant injection, and LL2 cell injection, respectively)⁷. Intrathecal injection of Wnt5a resulted in a rapid mechanical hypersensitivity that returned to control levels by 24 hours after the injection⁷. Intrathecal injection of siRNA against Ryk decreased Ryk mRNA levels in the spinal cord and significantly reduced mechanical hypersensitivity caused by Wnt5a injection, spared nerve injury, and Complete Freud's Adjuvant injection⁷.

The methods of the invention can also be used in the treatment of psychiatric disorders including, for example, schizophrenia, delusional disorder, schizoaffective disorder, schizopheniform, shared psychotic disorder, psychosis, paranoid personality disorder, schizoid personality disorder, borderline personality disorder, anti-social personality disorder, narcissistic personality disorder, obsessive-compulsive disorder, delirium, dementia, mood disorders, bipolar disorder, depression, stress disorder, panic disorder, agoraphobia, social phobia, post-traumatic stress disorder, anxiety disorder, and impulse control disorders (e.g., kleptomania, pathological gambling, pyromania, and trichotillomania).

In addition to the in vivo methods described above, the methods of the invention can be used to treat nerves ex vivo, which may be helpful in the context of nerve grafts or nerve transplants. Thus, the compounds provided herein can be useful as components of culture media for use in culturing nerve cells in vitro.

The antibodies or antibody fragments described herein can be optionally combined with or administered in concert with each other or other agents known to be useful in the treatment of the relevant disease or condition. Thus, in the treatment of ALS, for example, the compounds can be administered in combination with Riluzole (Rilutek), minocycline, insulin-like growth factor 1 (IGF-I), and/or methylcobalamin. In another example, in the treatment of Parkinson's disease, inhibitors can be administered with L-dopa, dopamine agonists (e.g., bromocriptine, pergolide, pramipexole, ropinirole, cabergoline, apomorphine, and lisuride), dopa decarboxylase inhibitors (e.g., levodopa, benserazide, and carbidopa), and/or MAO-B inhibitors (e.g., selegiline and rasagiline). In a further example, in the treatment of Alzheimer's disease, inhibitors can be administered with acetylcholinesterase inhibitors (e.g., donepezil, galantamine, and rivastigmine) and/or NMDA receptor antagonists (e.g., memantine). The combination therapies can involve concurrent or sequential administration, by the same or different routes, as determined to be appropriate by those of skill in the art. The invention also includes pharmaceutical compositions and kits including combinations as described herein.

In some embodiments, in the context of the invention, the terms “contact” or “contacting” are defined to mean any manner in which a compound is brought into a position where it can mediate, modulate, or inhibit the growth of a neuron. “Contacting” can comprise injecting a diffusable or non-diffusable substance into the neuron or an area adjacent a neuron. “Contacting” can comprise placing a nucleic acid encoding a compound into or close to a neuron or non-neuronal cell in a manner such that the nucleic acid is expressed to make the compound in a manner in which it can act upon the neuron. Those of skill in the art, following the teachings of this specification, will be able to contact neurons with substances in any manner.

The methods for modulating growth of a neuron may, in certain embodiments, be methods for stimulating growth of a neuron, methods for regenerating a damaged neuron, or methods for guiding growth of a neuron along the anterior-posterior axis. In other embodiments, the methods for modulating growth of a neuron are further defined as methods for directionally orienting axon growth of a neuron between the spinal cord and the brain.

In certain embodiments, the neuron is contacted with an anti-Ryk monoclonal antibody or antibody fragment that specifically binds to a binding domain of Wnt affecting a Wnt signaling pathway, and may further involve exposing the neuron to a gradient of the anti-Ryk monoclonal antibody or antibody fragment that specifically binds to a binding domain of Wnt affecting a Wnt signaling pathway. The gradient may be in the spinal cord, such as a decreasing anterior-posterior gradient within the spinal cord. In other embodiments, exposing the neuron to the gradient involves stimulating directionally-oriented axon growth of the neuron along the anterior-posterior axis. Any direction of axon growth is contemplated by the present invention. In certain embodiments, the axon growth is directed from the spinal cord to the brain, such as in the growth of neurons in ascending somatosensory pathways. In other embodiments, the axon growth is directed from the brain to the spinal cord, such as in the growth of neurons in descending motor pathways or other regulatory pathways. In further embodiments, the axon growth is directed along the spinothalamic pathway.

The present invention also includes methods of modulating growth of a neuron in a subject, including: (a) providing a composition that includes an anti-Ryk antibody or antibody fragment that specifically binds to a binding domain of Wnt affecting a Wnt signaling pathway; and a pharmaceutical preparation suitable for delivery to the subject; and (b) administering the composition to the subject. The methods for modulating neuron growth of the present invention contemplate measurement of neuronal growth by any known means, as discussed above. For example, the method of modulating neuron growth may be defined as a method of promoting growth and regeneration of a neuron in a subject, a method of promoting axon growth and regeneration in a subject, or a method of promoting directionally-oriented axon growth in a subject. Directionally-oriented axon growth may be along the anterior-posterior axis such as from the spinal cord to the brain, or from the brain to the spinal cord.

In yet another aspect or embodiment, the present disclosure provides for a method of preventing or treating a cancer or tumor in a subject having or being at risk of developing the cancer or tumor comprising administering to the subject an effective amount of the above isolated antibody or antibody derivative, the above immunoconjugate, the above bispecific molecule, the above pharmaceutical composition, the above nucleic acid, the above vector, or the above host cell, thereby preventing or treating the cancer or tumor in the subject.

The present methods can be used for preventing or treating any suitable cancer or tumor. For example, the present methods can be used for preventing or treating a cancer or tumor that is caused by or associated with overexpression of Ryk and/or Wnt5a in a subject.

In another example, the present methods can be used for preventing or treating glioma, glioblastoma multiforme (GBM), a lymphoma, a leukemia, a brain cancer, a multiple myeloma, a pancreatic cancer, cholangiocarcinoma (a bile duct cancer), a liver cancer, a stomach cancer, a breast cancer, a kidney cancer, a lung cancer, a colorectal cancer, a colon cancer, a prostate cancer, an ovarian cancer, a cervical cancer, a skin cancer, melanoma, an esophagus cancer, a head and neck cancer, a thymic cancer, gastric cancer, melanoma, prostate cancer, ovarian cancer, small cell lung cancer, or an atypical teratoid rhabdoid tumor. In some embodiments, the present methods can be used for preventing or treating low grade glioma. In some embodiments, the present methods can be used for preventing or treating T- and B-cell acute lymphoblastic leukemia or acute myeloid leukemia. In some embodiments, the present methods can be used for preventing or treating diffuse large B-cell lymphoma (DLBC). In some embodiments, the present methods can be used for preventing or treating thymoma (THYM).

In some embodiments, the present methods can be used for treating a cancer or tumor in a subject. In some embodiments, the present methods can be used for preventing a cancer or tumor in a subject.

The present methods can be used for preventing or treating a cancer or tumor in any suitable subject. For example, the present methods can be used for preventing or treating a cancer or tumor in a mammal or a human.

In yet another aspect or embodiment, the present disclosure provides for an use of an effective amount of the above isolated antibody or antibody derivative, the above immunoconjugate, the above bispecific molecule, the above nucleic acid, the above vector, or the above host cell for manufacturing a medicament for preventing or treating a cancer or tumor in a subject having or being at risk of developing the cancer or tumor.

In some embodiments and not wishing to be bound any particular mechanism or theory, dysregulation of important developmental signaling pathways frequently leads to the formation and progression of cancer. Wnt signaling which is critical for embryonic development and adult tissue homeostasis is a prime example of this. Deregulated Wnt signaling is highly associated with numerous tumors and may contribute to drug resistance and recurrence of cancers. Receptor-like tyrosine kinase, or related-to-receptor tyrosine kinase (Ryk) is one of the Wnt-binding receptor tyrosine kinases (RTKs) and appears to signal predominantly through non-canonical Wnt pathways. Ryk controls fundamental cellular processes, such as cell polarity and movement via regulation of the cytoskeleton. With the notion that cancer development shares many similarities with embryonic development, it is rationally suspected that dysregulated Wnt/Ryk signaling plays a potential role in the pathogenesis of cancer, particularly in tissues in which Ryk is developmentally important.

Indeed, overexpression of Ryk and Wnt5a was found in glioma¹, a type of tumor that occurs in the brain and spinal cord, and their expression levels correlated with the histological grades of glioma tissues¹. In vitro knockdown and overexpression experiments demonstrated that Ryk is critical for the migration, invasion and anchorage-independent growth of glioma cells^{1,2}. In addition, Wnt5a/Ryk signaling was reported to promote the resistance of melanoma cells to targeted BRAF inhibition³. High expression of Ryk was also described in T- and B-cell acute lymphoblastic leukemia and acute myeloid leukemia⁴. See References below:

### References:

(1) Habu, M.; Koyama, H.; Kishida, M.; Kamino, M.; Iijima, M.; Fuchigami, T.; Tokimura, H.; Ueda, M.; Tokudome, M.; Koriyama, C.; et al. Ryk Is Essential for Wnt-5a-Dependent Invasiveness in Human Glioma. J. Biochem. (Tokyo) 2014, 156 (1), 29-38. https://doi.org/10.1093/jb/mvu015.
(2) Adamo, A.; Fiore, D.; De Martino, F.; Roscigno, G.; Affinito, A.; Donnarumma, E.; Puoti, I.; Vitiani, L. R.; Pallini, R.; Quintavalle, C.; et al. RYK Promotes the Stemness of Glioblastoma Cells via the WNT/β-Catenin Pathway. Oncotarget 2017, 8 (8). https://doi.org/10.18632/oncotarget.14564.
(3) Anastas, J. N.; Kulikauskas, R. M.; Tamir, T.; Rizos, H.; Long, G. V.; von Euw, E. M.; Yang, P.-T.; Chen, H.-W.; Haydu, L.; Toroni, R. A.; et al. WNT5A Enhances Resistance of Melanoma Cells to Targeted BRAF Inhibitors. J. Clin. Invest. 2014, 124 (7), 2877-2890. https://doi.org/10.1172/JCI70156.
(4) Alvarez-Zavala, M.; Riveros-Magaña, A. R.; García-Castro, B.; Barrera-Chairez, E.; Rubio-Jurado, B.; Garcés-Ruiz, O. M.; Ramos-Solano, M.; Aguilar-Lemarroy, A.; Jave-Suarez, L. F. WNT Receptors Profile Expression in Mature Blood Cells and Immature Leukemic Cells: RYK Emerges as a Hallmark Receptor of Acute Leukemia. Eur. J. Haematol. 2016, 97 (2), 155-165. https://doi.org/10.1111/ejh.12698.

As described herein, the disclosed methods can be carried out in vivo, such as in the treatment of neurodegenerative diseases, neurological disorders or injuries to the nervous system. The methods can also be carried out in vitro or ex vivo, such as in laboratory studies of neuron function and in the treatment of nerve grafts or transplants. Accordingly, in some embodiments, the neuron forms part of a nerve graft or a nerve transplant. In some embodiments, the neuron is ex vivo or in vitro. In some embodiments, the nerve graft or the nerve transplant forms part of an organism, human or non-human (e.g., mammal, primate, rat, mouse, rabbit, bovine, dog, cat, pig, etc.).

In another aspect or embodiment, the invention provides a composition comprising the antibody or antibody fragment of the invention, which can be prepared for administration to a subject by mixing the antibody or immunogenic peptide fragment with physiologically acceptable carriers or excipients. Such carriers will be nontoxic to recipients at the dosages and concentrations employed. Ordinarily, the preparation of such compositions entails combining the particular antibody with saline, buffers, antioxidants such as ascorbic acid, low molecular weight (less than about 10 residues) polypeptides, proteins, amino acids, carbohydrates including glucose or dextrans, or chelating agents such as EDTA, glutathione and other stabilizers and excipients. Such compositions can be in suspension, emulsion or lyophilized form and are formulated under conditions such that they are suitably prepared and approved for use in the desired application.

A physiologically acceptable carrier or excipient can be any material that, when combined with an immunogenic peptide or a polynucleotide of the invention, allows the ingredient to retain biological activity and does not undesirably disrupt a reaction with the subject's immune system. Examples include, but are not limited to, any of the standard physiologically acceptable carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well-known conventional methods (see, for example, Remington's Pharmaceutical Sciences, Chapter 43, 14th Ed., Mack Publishing Co., Easton Pa. 18042, USA).

For administration to a subject, a peptide, or an encoding polynucleotide, generally is formulated as a composition. Accordingly, the present invention provides a composition, which generally contains, in addition to the peptide or polynucleotide of the invention, a carrier into which the peptide or polynucleotide can be conveniently formulated for administration. For example, the carrier can be an aqueous solution such as physiologically buffered saline or other solvent or vehicle such as a glycol, glycerol, an oil such as olive oil or an injectable organic esters. A carrier also can include a physiologically acceptable compound that acts, for example, to stabilize the peptide or encoding polynucleotide or to increase its absorption. Physiologically acceptable compounds include, for example, carbohydrates, such as glucose, sucrose or dextrans, antioxidants, such as ascorbic acid or glutathione, chelating agents, low molecular weight proteins or other stabilizers or excipients. Similarly, a cell that has been treated in culture for purposes of the practicing the methods of the invention, for example, synovial fluid mononuclear cells, dendritic cells, or the like, also can be formulated in a composition when the cells are to be administered to a subject.

It will be recognized to the skilled clinician that choice of a carrier or excipient, including a physiologically acceptable compound, depends, for example, on the manner in which the peptide or encoding polynucleotide is to be administered, as well as on the route of administration of the composition. Where the composition is administered under immunizing conditions, i.e., as a vaccine, it generally is administered intramuscularly, intradermally, or subcutaneously, but also can be administered parenterally such as intravenously, and can be administered by injection, intubation, or other such method known in the art. Where the desired modulation of the immune system is tolerization, the composition preferably is administered orally, or can be administered as above.

Pharmaceutically acceptable carriers useful for formulating an agent for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally, intranasally or any other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

The total amount of a compound or composition, e.g., an anti-Ryk antibody, to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the plasma expander used to treat blood loss in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

D. Methods for Assessing a Ryk Polypeptide in a Sample

In yet another aspect or embodiment, the present disclosure provides for a method for assessing a Ryk polypeptide in a sample, which method comprises: a) contacting a sample containing or suspected of containing a Ryk polypeptide with the above isolated antibody or antibody derivative, the above immunoconjugate, or the above bispecific molecule; and b) assessing binding between the Ryk polypeptide, if present in the sample, and the isolated antibody or antibody derivative, the immunoconjugate or the bispecific molecule to assess the presence, absence, level or amount of the Ryk polypeptide in the sample.

The present methods can be used for assessing a Ryk polypeptide in any suitable sample. For example, the present methods can be used for assessing a Ryk polypeptide in a liquid, a semi-liquid or a solid sample. In another example, the present methods can be used for assessing a Ryk polypeptide in a biological sample. In some embodiments, the biological sample is a blood or a urine sample. In some embodiments, the blood sample is a serum, a plasma or a whole blood sample. In some embodiments, the sample is a clinical sample, e.g., a tissue biopsy sample.

The present methods can be used for assessing any suitable Ryk polypeptide. For example, the present methods can be used for assessing a natural Ryk polypeptide, protein or a fragment thereof in a sample.

The present methods can be conducted in any suitable manner or format. In some embodiments, the Ryk polypeptide is contacted with the above isolated antibody or antibody derivative. In some embodiments, the Ryk polypeptide is contacted with the above immunoconjugate. In some embodiments, the Ryk polypeptide is contacted with the above bispecific molecule. In some embodiments, the binding between the Ryk polypeptide, if present in the sample, and the isolated antibody or antibody derivative, the immunoconjugate or the bispecific molecule is assessed by a format selected from the group consisting of an enzyme-linked immunosorbent assay (ELISA), immunoblotting, immunoprecipitation, radioimmunoassay (RIA), immune-staining, latex agglutination, indirect hemagglutination assay (IHA), complement fixation, indirect immunofluorescent assay (IFA), nephelometry, flow cytometry assay, surface plasmon resonance (SPR), chemiluminescence assay, lateral flow immunoassay, u-capture assay, inhibition assay and avidity assay.

In some embodiments, the present methods are used to assess the presence or absence of the Ryk polypeptide in the sample, In some embodiments, the present methods are used to assess the level or amount of the Ryk polypeptide in the sample.

The present methods can be used for assessing a Ryk polypeptide in any suitable sample. For example, the sample can be isolated or derived from a subject. The subject can be a mammal or a human.

The present methods can be used for any suitable purposes. For example, the present methods can be used for diagnosis, prognosis, stratification, risk assessment, or treatment monitoring of a disease, disorder or injury associated with abnormal level or amount of the Ryk polypeptide in a subject. The assessed level or amount of the Ryk polypeptide can be compared with a threshold value or range to assess whether a level or amount of the Ryk polypeptide in a subject is normal or abnormal. Any suitable threshold value or range can be used in comparison. For example, the threshold value or range can be obtained or derived from a subject or a population of subjects that have the disease, disorder or injury, a subject or a population of subjects that do not have the disease, disorder or injury, or a subject or a population of subjects that are treated, cured or recovered from the disease, disorder or injury.

In some embodiments, the present methods are used for diagnosis, prognosis, stratification, risk assessment, or treatment monitoring of a disease, disorder or injury associated with abnormally low level or amount of the Ryk polypeptide in a subject. In some embodiments, the present methods are used for diagnosis, prognosis, stratification, risk assessment, or treatment monitoring of a disease, disorder or injury associated with abnormally high level or amount of the Ryk polypeptide in a subject. In some embodiments, the present methods are used for diagnosis, prognosis, stratification, risk assessment, or treatment monitoring of degeneration of a neuron, a neurological disease, disorder or injury, a tumor or a cancer.

The present methods can further comprise treating a subject for the disease, disorder or injury. In some embodiments, the treatment comprises modulating or adjusting level or amount of the Ryk polypeptide in the subject.

### E. EXAMPLES

### Example 1

### Humanisation and Deimmunisation of Ab5.5

### Summary

The antibody Ab5.5 disclosed in WO 2017/172733 A1 was humanized and deimmunized using Epibase™ and in silico tools to. The preferred Acceptor framework for the grafting of the complementarity determining regions (CDRs) was selected from the set of human germlines and a structural model for the Fv-region of the antibody was constructed using Lonza Biologics molecular modelling platform. The CDR-grafting was accomplished by substituting any mismatched residues between the Parental and Acceptor frameworks. Substitutions at potentially critical positions such as those in the Vernier zone, the VH/VL inter-chain interface or at positions determining the CDR canonical class were analysed for prospective back mutations. Epibase™ v.4.0 immunoprofiling of Ab5.5 against 85 HLA class II allotypes in the Global set was performed on the sequences. Predicted epitopes were evaluated for deimmunising substitutions that would be considered effective in reducing the potential immunogenicity.

A total of 15 humanised/deimmunised variant sequences were recommended to VersaPeutics for further characterization.

### Introduction to Humanisation and Deimmunisation

Humanisation by CDR grafting is a proven, successful technique to take antibodies originating from murine, other xenogenic species or hybridomas and reduce the potential immunogenicity whilst retaining the binding and functional activity of the Parental antibody. Commonly starting from a chimeric antibody, the aim is to remove the foreign framework regions (FR) in the variable domains that can evoke an immune response (Bruggemann et al. 1987). The solution to the problem is to “graft” the CDRs of the murine antibody onto a human Acceptor framework (Jones et al. 1986).

However, CDR-grafting alone can lead to a significant reduction or complete loss of binding affinity, as a set of supporting framework residues in the Vernier zone are important for maintaining the conformation of the CDRs (Foote and Winters 1992). This problem can be solved by reintroducing murine residues into the human framework (Queen et al. 1989); such substitutions are commonly called back-mutations.

Most therapeutic proteins are, to a varying extent, immunogenic (Van Walle et al. 2007, Stas et al. 2009) and even so called fully-human antibody therapeutics may contain immunogenic regions (Harding et al. 2010). Immunogenicity is the ability to induce a Th (T-helper) response, which is triggered when a unique T-cell receptor recognises a peptide bound to the HLA class II molecules displayed on antigen presenting cells. The peptides are generated from proteins internalised by the antigen presenting cell which are then processed through the endosomal cleavage pathway. Only peptides with sufficient affinity for the HLA class II molecules will be presented on the cell surface, and could possibly trigger a Th response.

Consequently, it is possible to lower the immunogenicity potential by removing Th epitopes, a process known as deimmunisation (Chamberlain 2002, Baker and Jones 2007). This is achieved by predicting which peptides in the therapeutic protein can bind to HLA class II molecules, and subsequently introduce substitutions that eliminate or reduce the peptide binding affinity for HLA class II molecules.

There are several HLA class II genes and almost all are highly polymorphic. Additionally, HLA class II molecules consist of an alpha and beta chain, each derived from a different gene which, with the inherent polymorphism, further increases variation. Specifically, every individual expresses the genes: DRA/DRB, DQA/DQB and DPA/DPB. Of these only DRA is non-polymorphic. In addition, a ‘second’ DRB gene (DRB3, DRB4 or DRB5) may also be present, the product of which also associates with the DRA chain.

The focus during a deimmunisation is on the DR allotypes, which are known to express at a higher level than DQ and DP (Laupeze et al. 1999, Gansbacher and Zier 1988, Berdoz et al. 1987, Stunz et al. 1989). DR allotypes are usually referred to by the DRB gene as the DRA gene remains constant, for example DRB1*01:01, where the digits are allele-specific.

The assessment of severity for individual epitopes is based on the criteria of promiscuity, e.g., the number of HLA allotypes a specific epitope binds to, as well as the importance (frequency) of the allotypes in the population and a qualitative assessment of the HLA:peptide complex binding strength. As the T-cell population of an individual has been selected to not recognise ‘self-peptides’ it is possible to screen the protein that is being deimmunised for peptides that correspond to (known) self-peptides which should not normally induce a Th response. Though it is not known in detail which endogenous proteins are internalised during T cell maturation and as such give rise to self-peptides, antibodies are among them (Kirschmann et al. 1995, Verreck et al. 1996, Harding et al. 2010).

As the most significant property of a therapeutic antibody is the activity, it is important that substitutions proposed during the humanisation and deimmunisation do not affect the affinity or stability of the antibody. A large amount of information has been collected in the last 20 years on humanisation and grafting of the CDRs (Jones et al. 1986, Foote and Winters 1992), the biophysical properties of antibodies (Ewert et al. 2003), the conformation of the CDR-loops (Chothia and Lesk 1987, Al-Lazikani et al. 1997, North et al. 2011) and for the frameworks (Vargas-Madrazo and Paz-Garcia 2003, Honegger et al. 2009), which along with advances in protein modelling (Desmet et al. 2002, Almagro et al. 2011) makes it possible to accurately humanise and deimmunise antibodies with retained binding affinity and stability.

### Sequence Analysis

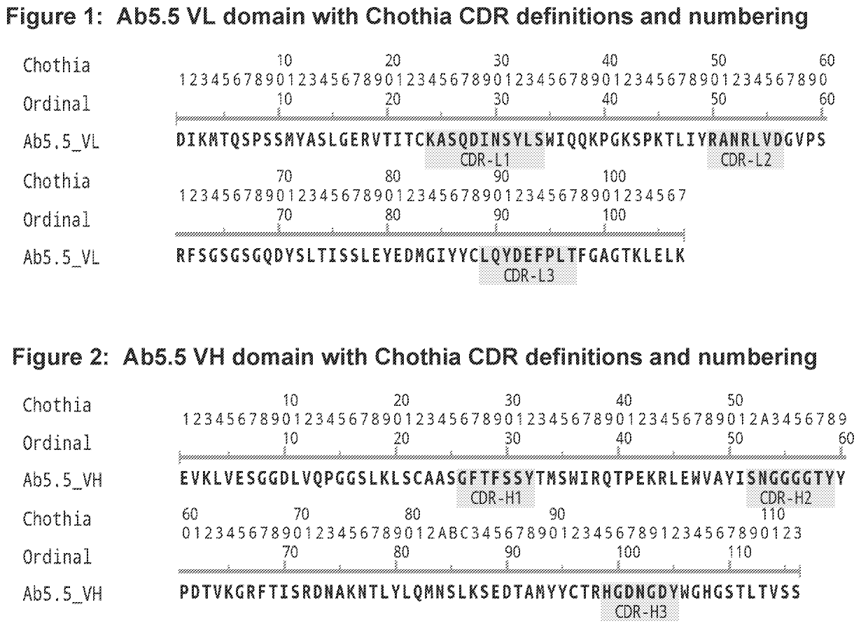

Analysis of the domain content of Ab5.5 showed it to be a murine IgG antibody. Variable domain boundaries were determined along with the boundaries of the complementarity-determining regions (CDRs) according to several commonly used definitions (Kabat and Wu 1991, Chothia and Lesk 1987 updated in Al-Lazikani et al. 1997, Honegger and Plückthun 2001). The updated Chothia CDR definition (Al-Lazikani et al. 1997) will be used as reference throughout the report. This definition differs from the original Chothia and Lesk 1987 publication by the inclusion of the heavy chain Chothia positions H:57 and H:58 in the CDR H2 definition. Positional numbering is ordinal unless otherwise specified, in which case Chothia 1987 numbering will be used. The variable domains of Ab5.5 were isolated and annotated with Chothia CDR definitions and are shown in FIG. 1 and FIG. 2.

### Optimal Acceptor Framework Selection

Sequence alignments comparing Ab5.5 variable domains to the human germlines were generated. Based on overall sequence identity, matching interface positions and similarly classed CDR canonical positions, a germline family was identified for each of the light and heavy chains as containing the most promising Acceptor frameworks, VK1 for the light chain and VH3 for the heavy chain. Ab5.5 was found to be most compatible with the light chain germline VK1-L1 and heavy VH3-3-07. An alignment of the variable domains to the 10 most similar genes in each family and the most similar J-segment can be found in Appendix 10.1.

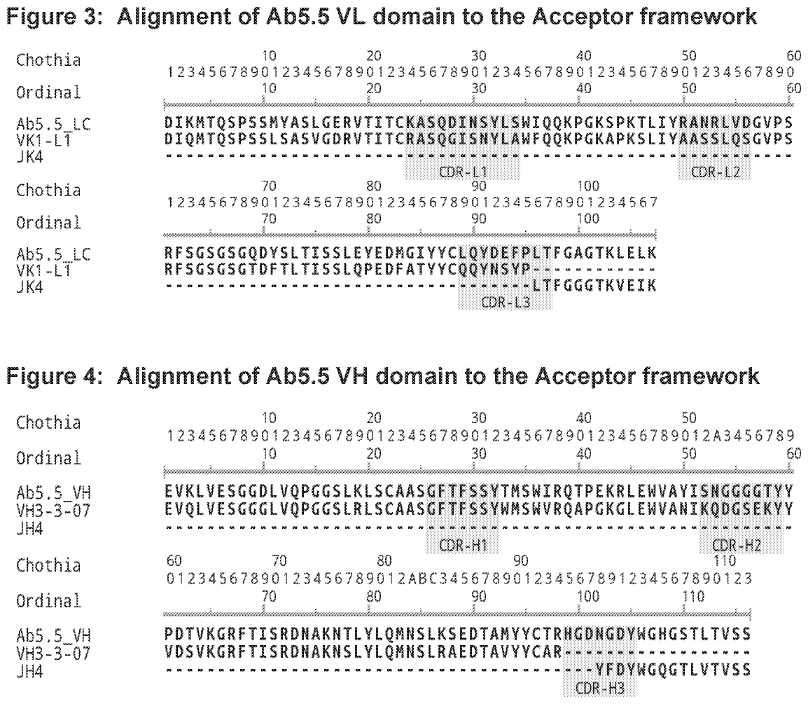

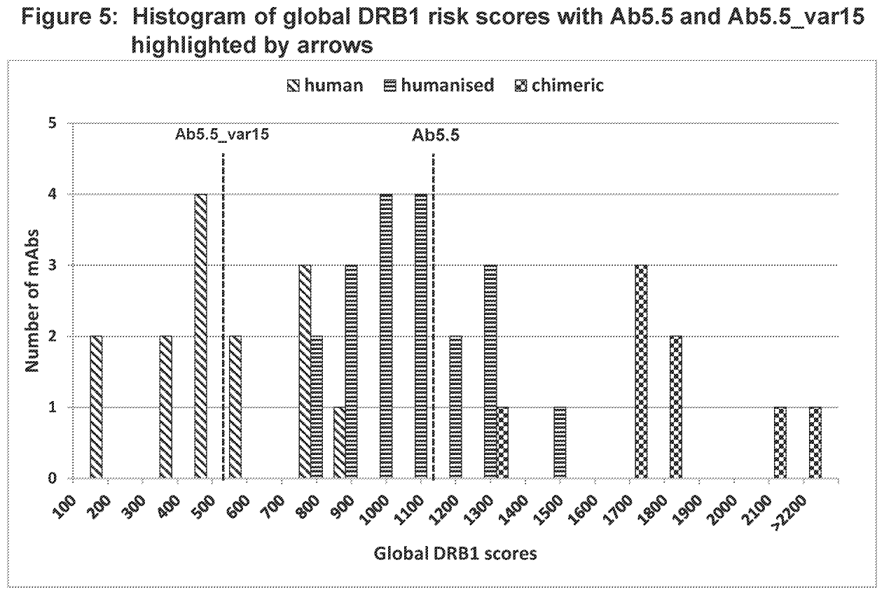

The J-segment genes were compared to the Parental sequence over FR4 and J-segments JK4 and JH4 were selected for the light and heavy chains, respectively. An alignment of the Parental sequences to the Acceptor framework is given in FIG. 3 and FIG. 4 below.

### Engineered Chains

In silico humanization, deimmunization, and protein engineering of the parental heavy and light chain sequences was performed. A total of three (3) humanised/deimmunised light chains and five (5) humanised/deimmunised heavy chains were designed (See Table 1 below).

TABLE 1

| Chain | Name | Description |
|---|---|---|
| L | Ab5.5_VL_1 | Humanised chain |
| L | Ab5.5_VL_2 | Deimmunised chain with L:V19A and L:A100G |
| | | substitutions. |
| L | Ab5.5_VL_3 | Engineered chain with L:D56E substitution |
| | | to remove isomerisation/fragmentation risk. |
| H | Ab5.5_VH_1 | Conservatively humanised chain retaining |
| | | parental H:Thr78 and H:His108. |
| H | Ab5.5_VH_2 | Humanised chain with H:T78S and H:H108Q |
| | | substitutions. |
| H | Ab5.5_VH_3 | Engineered chain with H:N53T and H:N102S |
| | | substitutions to remove deamidation risks. |
| H | Ab5.5_VH_4 | Engineered chain with H:N53T and H:N102Q |
| | | substitutions to remove deamidation risks. |
| H | Ab5.5_VH_5 | Deimmunised chain with H:V48A substitution. |

Table of engineered chains comprising humanised and deimmunised chains

### Engineered Chain Sequences

The sequences of the engineered chains are displayed below with CDR positions highlighted in grey.

Humanised and Deimmunised Light Chain Sequences
Ab5.5_VL (SEQ ID NO: 9)

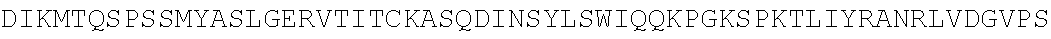

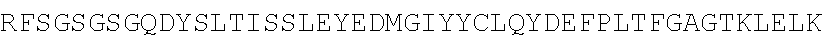

Ab5.5_VL_1 (SEQ ID NO: 11)

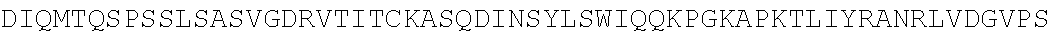

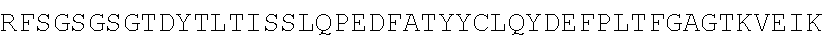

Ab5.5_VL_2 (SEQ ID NO: 12)

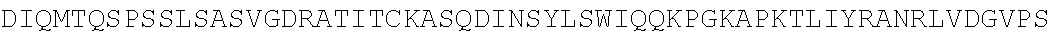

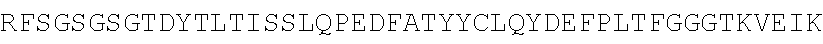

Ab5.5_VL_3 (SEQ ID NO: 13)

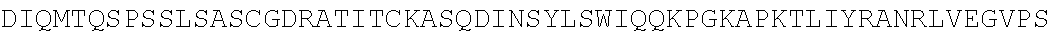

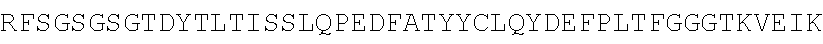

Humanised and Deimmunised Heavy Chain Sequences
Ab5.5_VH (SEQ ID NO: 10)

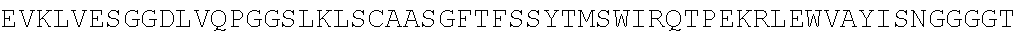

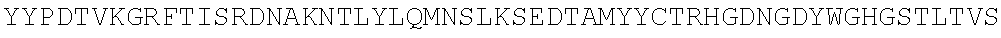

S
Ab5.5_VH_1 (SEQ ID NO: 14)

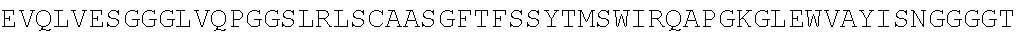

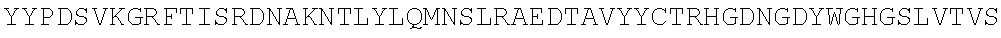

S
Ab5.5_VH_2 (SEQ ID NO: 15)

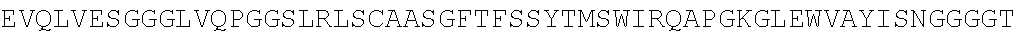

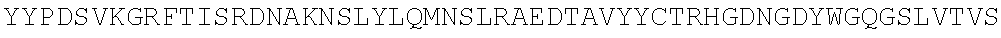

S
Ab5.5_VH_3 (SEQ ID NO: 16)

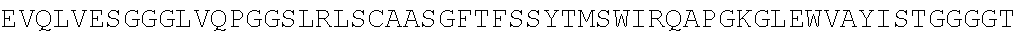

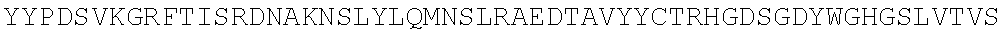

S
Ab5.5_VH_4 (SEQ ID NO: 17)

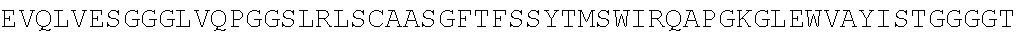

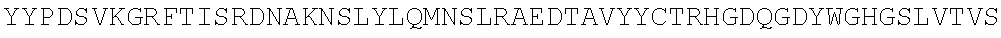

S
Ab5.5_VH_5 (SEQ ID NO: 18)

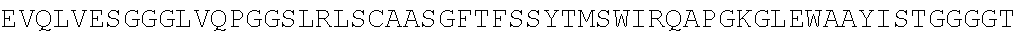

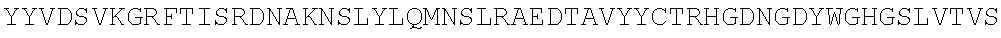

S

### Sequence Alignments

Sequence alignment of the variable regions of the heavy and light chains were performed using Clustal Omega (version 1.2.4). CDR positions are highlighted in grey and amino acids differing from the parental chain are colored.

Alignment of Parental VL sequence to Humanised/Deimmunised chains

| | | |
|---|---|---|
| Ab5.5_VL | 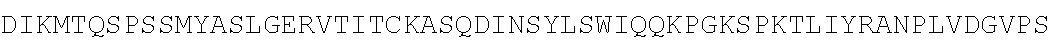 | 60 |
| Ab5.5_VL_1 | 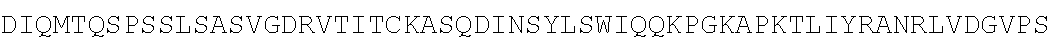 | 60 |
| Ab5.5_VL_2 | 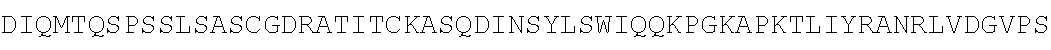 | 60 |
| Ab5.5_VL_3 | 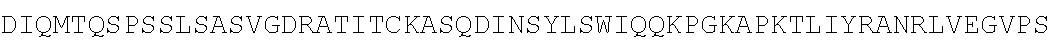 | 60 |
| | **:*******:**:*:*:***********************:************:**** | |
| Ab5.5_VL | 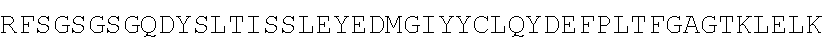 | 107 |
| Ab5.5_VL_1 | 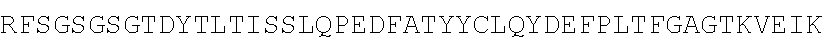 | 107 |
| Ab5.5_VL_2 | 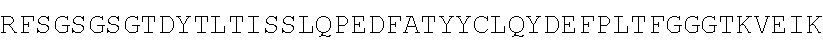 | 107 |
| Ab5.5_VL_3 | 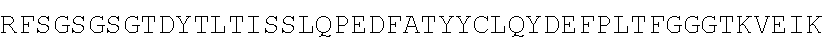 | 107 |
| | ******** **:******: **: **************: ***:*:* | |

Alignment of Parental VH sequence to Humanised/Deimmunised chains

| | | |
|---|---|---|
| Ab5.5_VH | 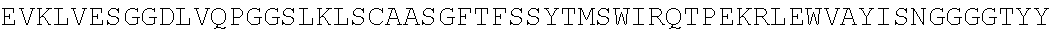 | 60 |
| Ab5.5_VH_1 | 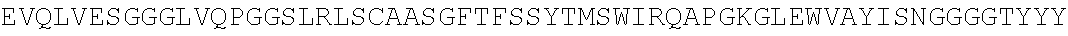 | 60 |
| Ab5.5_VH_2 | 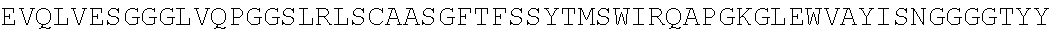 | 60 |
| Ab5.5_VH_3 | 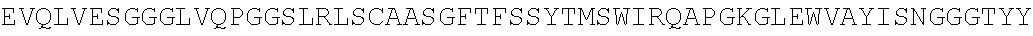 | 60 |
| Ab5.5_VH_4 | 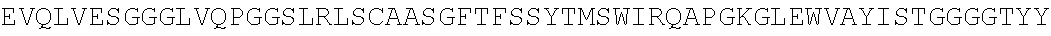 | 60 |
| Ab5.5_VH_5 | 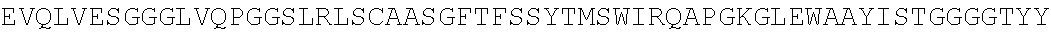 | 60 |
| | **:******:********:********************:* * ***:****:******* | |
| Ab5.5_VH | 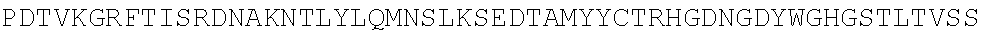 | 116 |
| Ab5.5_VH_1 | 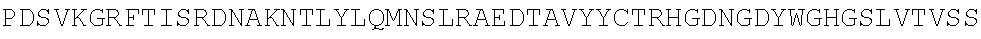 | 116 |
| Ab5.5_VH_2 | 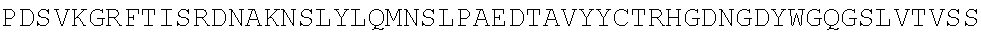 | 116 |
| Ab5.5_VH_3 | 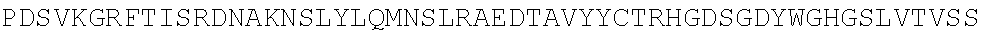 | 116 |
| Ab5.5_VH_4 | 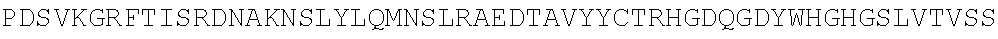 | 116 |
| Ab5.5_VH_5 | 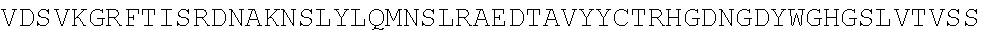 | 116 |
| | *:**************:********::****:********:*****:** :**** | |

### Sequence Percent Identity

The engineered sequences were individually aligned to the parental sequence using the Needleman and Wunsch algorithm (Needleman and Wunsch 1970). The alignment and calculations were performed using the EMBOSS Needle web tool (Madeira et al. 2019)(matrix=BLOSUM62, gap open=10, gap extend=0.5, end gap penalty=false, end gap open=10, end gap extend=0.5). The Table 2 below shows the percent identity for the indicated comparisons.

TABLE 2

| | Ab5.5_VH_1 | Ab5.5_VH_2 | Ab5.5_VH_3 | Ab5.5_VH_4 | Ab5.5_VH_5 |
|---|---|---|---|---|---|
| Ab5.5_VH | 89.7 | 87.9 | 87.1 | 87.1 | 86.2 |

| | Ab5.5_VL_1 | Ab5.5_VL_2 | Ab5.5_VL_3 |
|---|---|---|---|
| Ab5.5_VL | 86 | 84.1 | 83.2 |

### Variant Combinations

Fifteen variants were designed based on the possible combinations of the humanized/deimmunized heavy and light chains (See Table 3 below).

TABLE 3

| Variant Name | Light Chain Name | Heavy Chain Name | Comment |
|---|---|---|---|
| Ab5.5 | Ab5.5_VL | Ab5.5_VH | Parental |
| Ab5.5_var1 | Ab5.5_VL_1 | Ab5.5_VH_1 | Humanised Light chain, conservatively humanised |
| | | | Heavy chain retaining parental H:Thr78 and |
| | | | H:His108. |
| Ab5.5_var2 | Ab5.5_VL_1 | Ab5.5_VH_2 | Humanised Light chain, humanised Heavy chain |
| | | | with H:T78S and H:H100Q substitutions. |
| Ab5.5_var3 | Ab5.5_VL_1 | Ab5.5_VH_3 | Humanised Light chain, engineered Heavy chain |
| | | | with H:N53T and H:N102S substitutions. |
| Ab5.5_var4 | Ab5.5_VL_1 | Ab5.5_VH_4 | Humanised Light chain, engineered Heavy chain |
| | | | with H:N53T and H:N102Q substitutions. |
| Ab5.5_var5 | Ab5.5_VL_1 | Ab5.5_VH_5 | Humanised Light chain, deimmunised Heavy chain |
| | | | with H:V48A substitution. |
| Ab5.5_var6 | Ab5.5_VL_2 | Ab5.5_VH_1 | Deimmunised Light chain, conservatively |
| | | | humanised Heavy chain retaining parental H:Thr78 |
| | | | and H:His108. |
| Ab5.5_var7 | Ab5.5_VL_2 | Ab5.5_VH_2 | Deimmunised Light chain, humanised Heavy chain |
| | | | with H:T78S and H:H100Q substitutions. |
| Ab5.5_var8 | Ab5.5_VL_2 | Ab5.5_VH_3 | Deimmunised Light chain, engineered Heavy chain |
| | | | with H:N53T and H:N102S substitutions. |
| Ab5.5_var9 | Ab5.5_VL_2 | Ab5.5_VH_4 | Deimmunised Light chain, engineered Heavy chain |
| | | | with H:N53T and H:N102Q substitutions. |
| Ab5.5_var10 | Ab5.5_VL_2 | Ab5.5_VH_5 | Deimmunised Light chain, deimmunised Heavy |
| | | | chain with H:V48A substitution. |
| Ab5.5_var11 | Ab5.5_VL_3 | Ab5.5_VH_1 | Engineered Light chain, conservatively humanised |
| | | | Heavy chain retaining parental H:Thr78 and |
| | | | H:His108. |
| Ab5.5_var12 | Ab5.5_VL_3 | Ab5.5_VH_2 | Engineered Light chain, humanised Heavy chain |
| | | | with H:T78S and H:H100Q substitutions. |
| Ab5.5_var13 | Ab5.5_VL_3 | Ab5.5_VH_3 | Engineered Light chain, engineered Heavy chain |
| | | | with H:N53T and H:N102S substitutions. |
| Ab5.5_var14 | Ab5.5_VL_3 | Ab5.5_VH_4 | Engineered Light chain, engineered Heavy chain |
| | | | with H:N53T and H:N102Q substitutions. |
| Ab5.5_var15 | Ab5.5_VL_3 | Ab5.5_VH_5 | Engineered Light chain, deimmunised Heavy chain |
| | | | with H:V48A substitution. |

### Epibase™ Immunoprofiling Comparison

The most deimmunised/engineered variant combination of Ab5.5 (Ab5.5_var15) was taken through Epibase™ v.4.0 immunoprofiling. As the level of detail in the Epibase™ profiles is too granular to compare in detail, a comparison based on three types of immunoprofile statistics was performed between the Parental antibody and the humanised/deimmunised variants (5.12).

The predicted critical epitopes of DRB1, DRB3/4/5, DQ and DP epitopes for the humanised, deimmunised and parental sequences are shown in 4 below. Peptides binding to multiple allotypes of the same group were counted as one.

TABLE 4
Critical epitope counts per gene family

| Variant Name | DRB1 | DRB3/4/5 | DQ | DP |
|---|---|---|---|---|
| Ab5.5 | 47 (17) | 20 (10) | 2 (1) | 3 (0) |
| Ab5.5_var1 | 24 (36) | 10 (22) | 2 (4) | 3 (1) |
| Ab5.5_var2 | 24 (38) | 10 (22) | 2 (4) | 3 (1) |
| Ab5.5_var3 | 24 (38) | 10 (23) | 3 (4) | 3 (1) |
| Ab5.5_var4 | 24 (38) | 10 (23) | 3 (4) | 3 (1) |
| Ab5.5_var5 | 21 (41) | 10 (24) | 4 (3) | 3 (1) |
| Ab5.5_var6 | 23 (36) | 9 (22) | 2 (4) | 3 (1) |
| Ab5.5_var7 | 23 (38) | 9 (22) | 2 (4) | 3 (1) |
| Ab5.5_var8 | 23 (38) | 9 (23) | 3 (4) | 3 (1) |
| Ab5.5_var9 | 23 (38) | 9 (23) | 3 (4) | 3 (1) |
| Ab5.5_var10 | 20 (41) | 9 (24) | 4 (3) | 3 (1) |
| Ab5.5_var11 | 22 (36) | 10 (22) | 2 (4) | 3 (1) |
| Ab5.5_var12 | 22 (38) | 10 (22) | 2 (4) | 3 (1) |
| Ab5.5_var13 | 22 (38) | 10 (23) | 3 (4) | 3 (1) |
| Ab5.5_var14 | 22 (38) | 10 (23) | 3 (4) | 3 (1) |
| Ab5.5_var15 | 19 (41) | 10 (24) | 4 (3) | 3 (1) |

Critical epitope counts per gene family, peptides binding to multiple allotypes of the same group were counted as one. Numbers between brackets refer to additional self-epitopes.

The difference between the Parental and variant antibodies in Table 4 accounts for the complete removal of potential epitopes. Many epitopes, especially promiscuous epitopes binding multiple allotypes, are difficult to completely remove.

An approximate score expressing a worst-case immunogenic risk based on the critical Th epitopes of DRB1 is given for the Parental sequence and the humanised/deimmunised variants in Table 5 below. Note that the proposed substitutions have been evaluated not only on DRB1 but also on DRB3/4/5, DQ and DP as well as the position, substitution, risk category and suitability of combinations of substitutions.

TABLE 5

| Variant Name | DRB1 score |
|---|---|
| Ab5.5 | 1130.2 |
| Ab5.5_var1 | 619.0 |
| Ab5.5_var2 | 577.8 |
| Ab5.5_var3 | 581.5 |
| Ab5.5_var4 | 580.9 |
| Ab5.5_var5 | 529.7 |
| Ab5.5_var6 | 609.3 |
| Ab5.5_var7 | 568.1 |
| Ab5.5_var8 | 571.8 |
| Ab5.5_var9 | 571.2 |
| Ab5.5_var10 | 520.0 |
| Ab5.5_var11 | 607.5 |
| Ab5.5_var12 | 566.3 |
| Ab5.5_var13 | 570.0 |
| Ab5.5_var14 | 569.4 |
| Ab5.5_var15 | 518.2 |

### Example 2

### Peptide Array Epitope Mapping

### Methods

### Epitope Mapping

The following sequence from the human Ryk protein fragment (the human ECTO domain, amino acids 134-227 of the human Ryk protein) was used for epitope mapping:

(SEQ ID NO: 26)
DMPQVNISVQGEVPRTLSVFRVELSCTGKVDSEVMILMQLNLTVNSSKN
FTVLNFKRRKMCYKKLEEVKTSALDKNTSRTIYDPVHAAPTTSTR

The protein sequence was elongated by neutral GSGSGSG (SEQ ID NO:27) linkers at the C- and N-terminus to avoid truncated peptides. The elongated sequence was translated into linear 15 amino acid peptides with a peptide-peptide overlap of 14 amino acids and peptides were printed onto a custom microarray. Antibodies were diluted to 1 μg/ml, 10 μg/ml and 100 μg/ml and then incubated with the microarrays for 16 hours at 4° C. The microarrays were washed and then incubated with Goat anti-human IgG (Fc) DyLight680 (0.2 μg/ml) for 45 minutes at room temperature. A LI-COR Odyssey Imaging System was used to scan the microarrays and quantify the antibody binding at each peptide spot.

Quantification of spot intensities and peptide annotation were based on 16-bit gray scale tiff files. The PepSlide® Analyzer calculated the raw, foreground and background fluorescence intensities of each spot on the microarray. Median foreground intensities of the 2-4 replicate spots per microarray were corrected to zero if the spot-to-spot deviation was greater than 40%.

In cases where it was not clear if a certain amino acid contributed to antibody binding, the corresponding letters were written in italics.

### Results

### Epitope Mapping

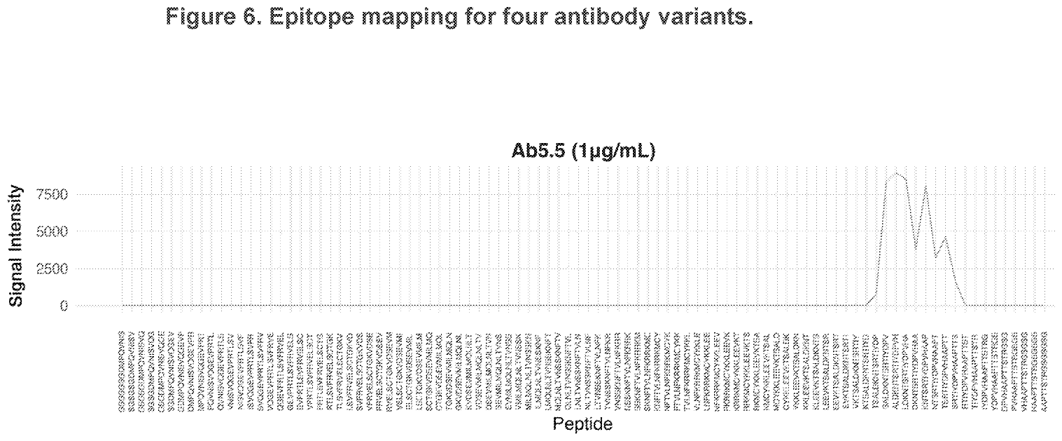

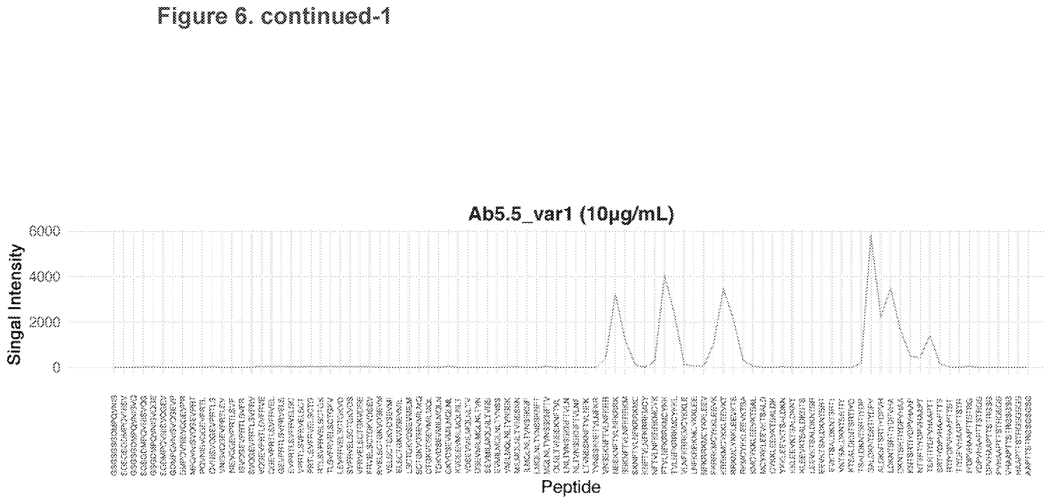

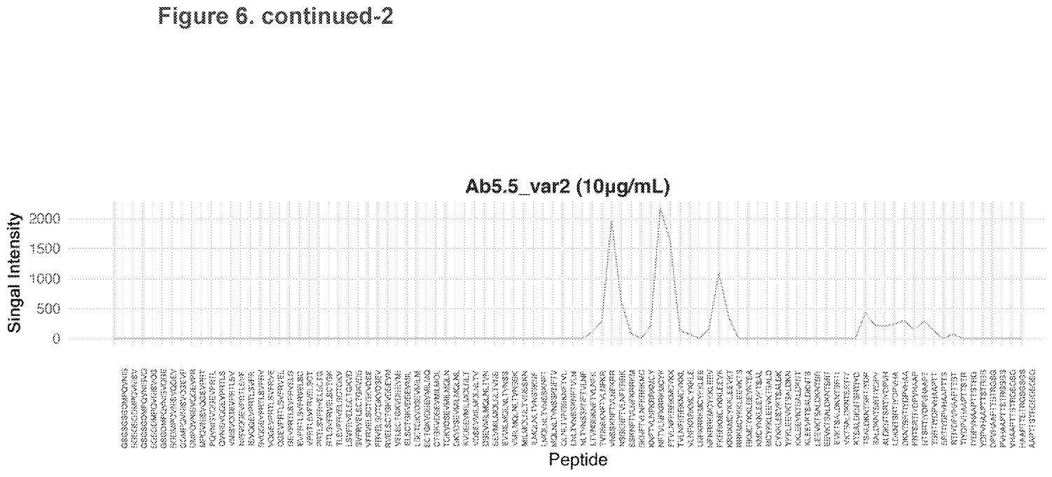

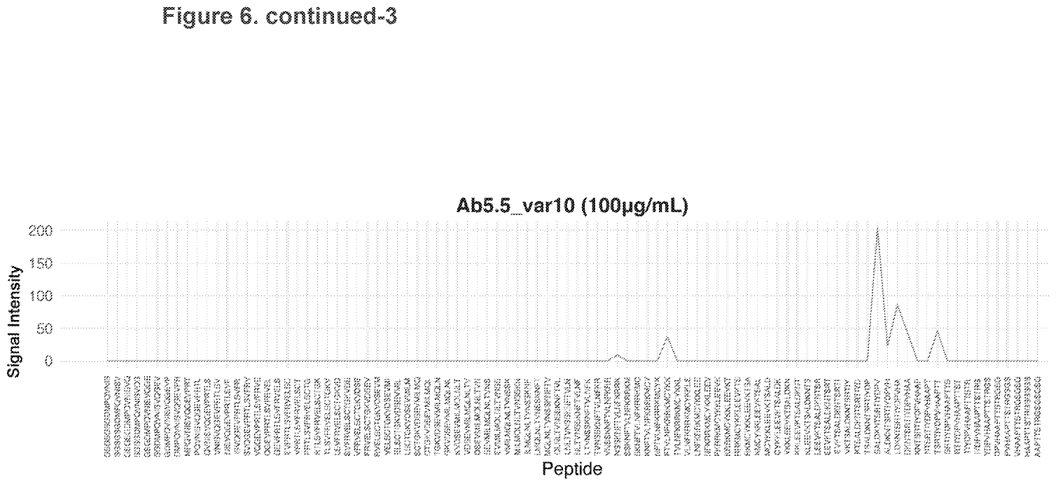

The amino acid sequences and other descriptions of the Ab5.5 variants, including Ab5.5_var1, Ab5.5_var2, and Ab5.5_var10, can be found in paragraphs [00263]-[00267] and Tables 1-3 disclosed above. The PEPperMAP ® Linear Epitope Mappings of Ab5.5, Ab5.5_var1, Ab5.5_var2, and Ab5.5_var10 are shown in FIG. 6. Pre-staining of a peptide microarray with secondary and control antibodies did not show any background interaction with the linear antigen-derived peptides that could interfere with the main assays. Incubation of other peptide microarray copies with the antibodies resulted in the following observations.

Ab5.5 showed a strong and clear monoclonal antibody response against two identical epitope-like spot patterns formed by adjacent peptides with the consensus motif TSRTIYDPV (SEQ ID NO:28). Ab5.5_var1 showed a moderate IgG response against two identical epitope-like spot patterns formed by adjacent peptides with the consensus motif TSRTIYDPV (SEQ ID NO:28); moreover, we observed additional interactions with peptides with the highly basic consensus motifs SSKNFTVLNFKRRK (SEQ ID NO:29), TVLNFKRRKMCYKK (SEQ ID NO:30) and RRKMCYKKLEEVK (SEQ ID NO:31) presumably due to non-specific ionic binding of the antibody. Ab5.5_var2 exhibited a similar but clearly weaker IgG response against two identical epitope-like spot patterns formed by adjacent peptides with the consensus motif TSRTIYDPV (SEQ ID NO:28); moreover, we also observed additional and even stronger interactions with peptides with the highly basic consensus motifs SSKNFTVLNFKRRK (SEQ ID NO:29), TVLNFKRRKMCYKK (SEQ ID NO:30) and RRKMCYKKLEEVK (SEQ ID NO:31) presumably due to non-specific ionic binding of Variant 2. Ab5.5_var10 showed a very weak response against two identical epitope-like spot patterns formed by adjacent peptides with the consensus motif TSRTIYDPV (SEQ ID NO:28); in addition, we observed even weaker presumably non-specific ionic interactions with single peptide NSSKNFTVLNFKRRK (SEQ ID NO:35) and peptides with the basic consensus motif VLNFKRRKMCYKK (SEQ ID NO:36).

All of the antibodies shared a proposed epitope based on peptides with the consensus motif TSRTIYDPV; the strongest response was found for Ab5.5, the weakest response for Ab5.5_var10; Ab5.5_var1 and Ab5.5_var2 further exhibited ionic interactions with highly basic peptides.

### Example 3

### Western Blot Screen of Ab5.5 Variants Using Recombinant Human Proteins

### Methods

### Plasmids

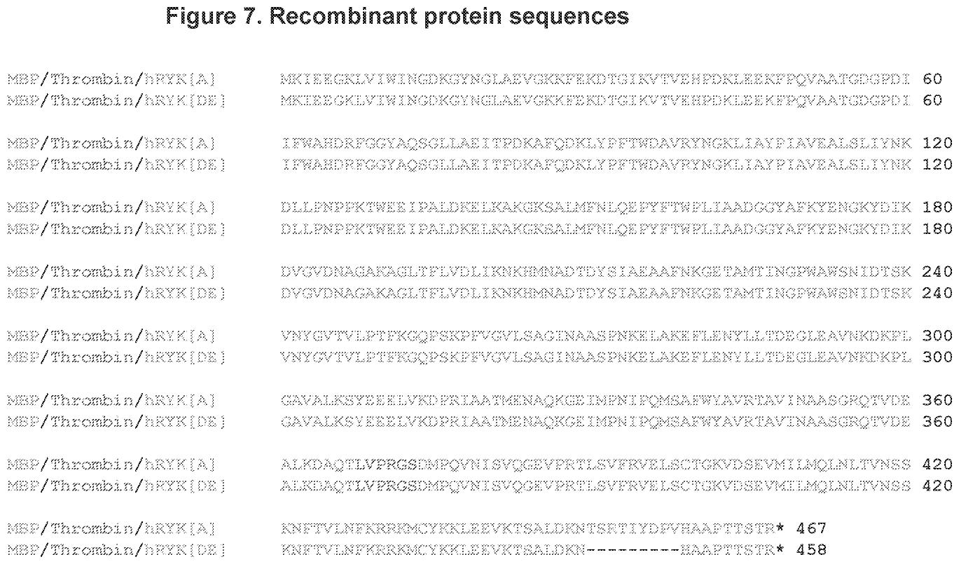

Plasmids were designed to express maltose-binding protein (MBP) fused to human Ryk (134-227) with (A, Antigen) and without (DE, Deleted Eptiope) the putative epitope discovered using peptide mapping (See FIG. 7 for sequence alignment).

### Transforming Bacteria

To transform bacteria, SHuffle T7 E. coli cells were thawed on ice for 10 minutes, then 30 μL of cell suspension was transferred to 2 pre-chilled tubes. 0.750 μL of antigen plasmid DNA and deleted epitope plasmid DNA were added to cell suspension and the tube was flicked 5 times to mix DNA with cells. The DNA and cell mixtures were incubated on ice for 30 minutes before a 45 second heat shock at 42° C. The mixtures were placed on ice for an additional 5 minutes. 950 μL of room temperature LB Broth (KD Medical BLE-3030) was added to the mixture, and then incubated at 37° C. for 60 minutes. After incubation, 500 μL of each transformed bacteria was added to an ampicillin selection plate, spread with ColiRollers (Novagen 71013-3) and incubated overnight at 37° C. After 24 hours incubation, 1 colony was chosen from each plate and placed into 10 mL LB Broth and incubated at 25° C. with 200 rpm shaking overnight. The following morning, each 10 mL culture was added to a flask containing 500 mL LB Broth and incubated at 25° C. with 200 rpm shaking until the measured OD was between 0.5 and 0.8. Transformed bacteria was then induced with 0.4 mM IPTG.

### Collecting Bacteria

LB Broth containing induced bacteria cells was collected and spun at 4,000×g for 10 minutes. Rinse pellet with 1×PBS and spin at 4,000×g for 10 minutes. Freeze at −80 ° C. or lyse pellet.

### Lysing Bacteria

To lyse bacteria, use 4 mL BPER (Thermo Scientific PI89821) with 1× Protease Inhibitor Cocktail (Thermo Scientific™ Halt™ Phosphatase Inhibitor Cocktail PI78420) per gram of bacteria. Lyse for 1 hour with gentle rocking. Collect lysate by spinning at 20,000×g for 10 minutes. Filter using a 45 micron filter.

### Protein Purification

For protein purification, amylose resin purchased from New England Biolabs was used with spin columns from Thermo Scientific. One (1) mL amylose resin was added to a 5 mL spin column and washed 3× with 4 mL Tris Buffer (50 mM Tris, 150 mM NaCl, 1 mM EDTA) by spinning at 500×g for 1 minute. Then, filtered bacteria lysates were gravity dripped through each column, and flow through was saved. The column was then washed 5× with 4 mL Tris buffer, spun at 500×g for 1 minute. To elute, 2 mL of elution buffer (10 mM maltose in Tris buffer) was added to the column and incubated for 2 minutes. The column was then spun at 500×g for 1 minute, collecting each elution. This was repeated 4 additional times.

### Sample Preparation and SDS-PAGE

The protein concentration of each sample was determined using the 660 nm protein assay with BSA standards. 4× Laemmli Sample Buffer (BIO-RAD Catalog #161-0747) was prepared by adding 0.1mL 2-Mercaptoethanol (#1610710) for every 0.9 mL of 4× sample buffer. 4× Laemmli Sample Buffer was then added to each sample in a 1:3 ratio. Samples were heated at 98° C. for 5 minutes in the heating block.

Two (2) μL of molecular weight marker (Precision Plus Protein™ All Blue Prestained Protein Standards #1610373) were used for each pair of antigen and deleted epitope samples. One hundred (100) ng of each sample were loaded into the gel. 4 gels were run at 200V for 35 minutes.

Use the Trans-Blot® Turbo™ Mini Nitrocellulose Transfer Packs (#1704158) in the Trans-Blot® Turbo™ Transfer System using the Mixed-MW protocol.

### Western Blot

Following transfer, membranes were dried and labeled using a black LI-COR pen. Membranes were reactivated with water, then blocked for 1 hour at room temperature using Odyssey PBS blocking buffer (Cat #) diluted 1:1 with 1× PBS. Following blocking, membranes were cut into 16 individual membranes and incubated overnight at 4° C. with either Chimera antibody or v1-v15 antibodies at a concentration of 1 μg/mL and MBP antibody (Cell Signaling) at a 1:4,000 dilution. After primary antibody incubation, membranes were rinsed 3× for 5 minutes with PBS-T and then incubated with anti-human (LiCOR) and anti-mouse (LiCOR) antibodies at a 1:15,000 dilution for 1 hour at room temperature. Membranes were then washed 3× for 5 minutes in PBS-T and the last PBS-T washed was exchanged with water. Membranes were then dried and scanned with a LI-COR CLx (Auto, 169 micron, medium quality, 0 offset).

### Quantification

For quantification, files were exported from the LI-COR CLx machine computer and transferred to a VersaPeutics computer. In Image Studio Lite, the analysis tools were utilized. The ‘Add Rectangle’ function was used to draw rectangles around each band to quantify with background set to ‘median, border width=3 and segment set to all.

### Results

Ab5.5 was raised against a 93 amino acid sequence of the mouse Ryk protein that contains two different amino acids than the human Ryk protein. To be an effective therapeutic protein in humans, it is essential that the humanized Ab5.5 variants recognize the human Ryk sequence. In order to confirm that the variants recognize the human protein, we performed a series of Western blot experiments using the recombinant fusion proteins shown in FIG. 7. One of the fusion proteins contains the human Ryk sequence corresponding to the original mouse antigen (human 134-227, mouse 118-211). The other protein is identical except that it lacks the putative epitope (TSRTIYDPV) (SEQ ID NO:28) for Ab5.5 that we discovered in a peptide mapping experiment.

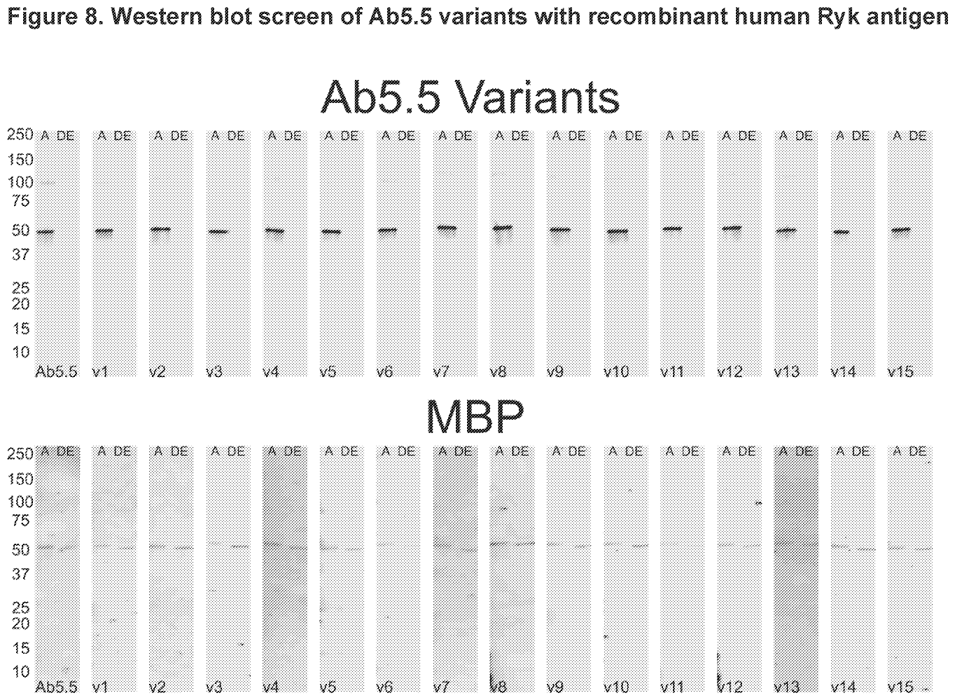

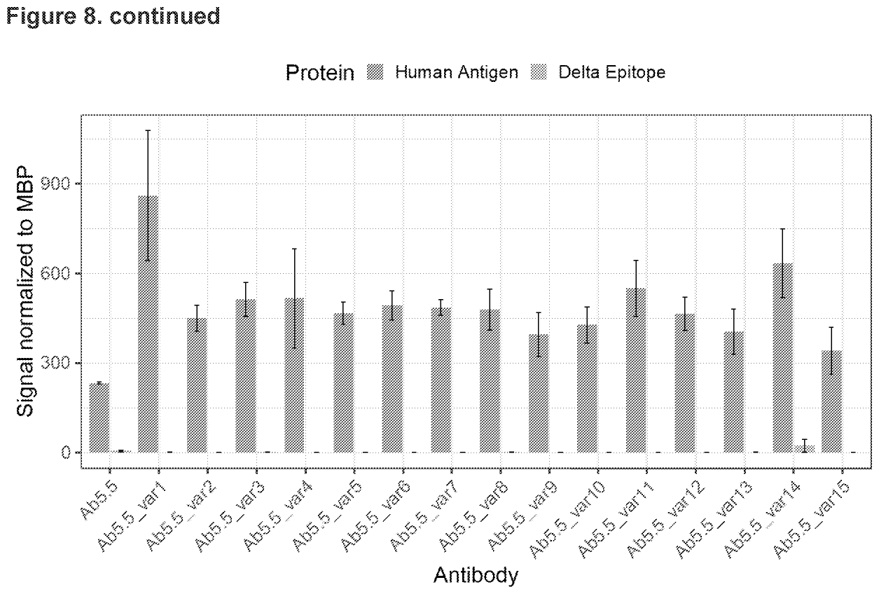

Three replicate Western blots using each of the Ab5.5 variants and the two purified proteins are shown in FIG. 8. These data demonstrate that all of the antibody variants strongly recognize the human Ryk protein sequence, and that the epitope identified by our mapping is essential for the binding of every variant. Importantly, the additional interactions identified by epitope mapping for variants 1, 2, and 10 are not required for binding to Ryk. It was also observed that variant 1 produced the strongest signal out of all the variants tested.

### Example 4

### Peptide Array Substitution Scans for Ab5.5 and Ab5.5_var1

### Methods

### Epitope Substitution Scan

A 15 amino acid sequence (¹LDKNTSRTIYDPVHA¹⁵) (SEQ ID NO:32) of the human Ryk protein (corresponding to amino acids 206-220) was selected for fine epitope mapping by substitution scan. Variants of ¹LDKNTSRTIYDPVHA¹⁵ (SEQ ID NO:32) with each amino acid position substituted by the 20 main amino acids were printed onto custom peptide microarrays. The resulting ¹LDKNTSRTIYDPVHA¹⁵ (SEQ ID NO:32) peptide microarrays contained 300 different peptide variants of the wild type peptides printed in triplicate (900 peptide spots) and were framed by additional HA (YPYDVPDYAG (SEQ ID NO:33), 80 spots) control peptides.

The microarrays were blocked with Rockland blocking buffer MB-070 for 30 minutes and then incubated with either Ab5.5 (1 μg/mL) or Ab5.5_var1 (100 μg/mL) for 16 hours at 4° C. The microarrays were washed and then incubated with Goat anti-human IgG (Fc) DyLight680 (0.1 μg/mL) and the control mouse monoclonal anti-HA (12CA5) DyLight800 (0.5 μg/mL) for 45 minutes at room temperature. A LI-COR Odyssey Imaging System was used to scan the microarrays and quantify the antibody binding at each peptide spot.

Quantification of spot intensities and peptide annotation were based on the 16-bit gray scale tiff files. The PepSlide® Analyzer calculated the raw, foreground and background fluorescence intensities of each spot on the microarray.

Heatmaps of the intensity values from the microarray scans were created and colored with black indicating the maximum intensity value and white indicating zero. Amino acid plots were created by dividing the spot intensity of a given artificial peptide by the spot intensity of the wild type peptide. The position of an amino acid thus reflected the intensity ratio compared to the amino acid of the native wild type peptide.

### Results

### Ab5.5

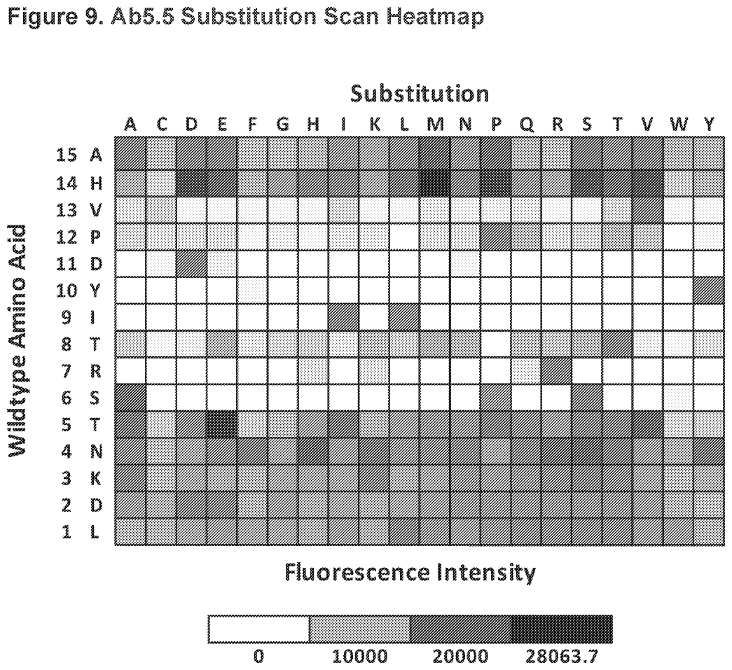

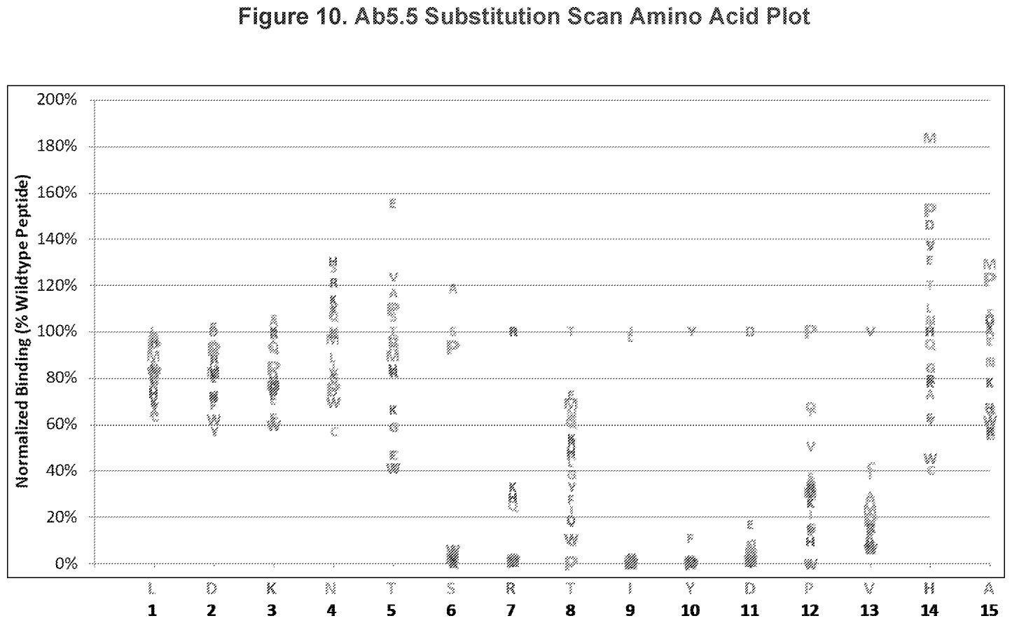

The heat map (FIG. 9) and the amino acid plot (FIG. 10) of Ab5.5 assayed against the substitution scan of wild type peptide ¹LDKNTSRTIYDPVHA¹⁵ (SEQ ID NO:32) highlighted a conserved core motif ⁶SRTIYDPV¹³ (SEQ ID NO:22) framed by N- and C-terminal variable stretches ¹LDKNT⁵ (SEQ ID NO:34) and ¹⁴HA¹⁵. This finding was in accordance with the previous epitope mapping against human Ryk amino acids 134-227.

Amino acid positions ¹⁰Y and ¹¹D were highly conserved, since an exchange by other amino acids resulted in at least 83% lower spot intensities and hence a reduction of antibody binding. Amino acid position ⁹I exhibited a high tolerance for a conserved exchange by L, but was also highly conserved and did otherwise not tolerate any other amino acid at all. Amino acid position ⁶S showed a similar tolerance for substitution by A and P, but was otherwise also highly conserved. Amino acid positions ⁷R and ¹³V were well conserved, since replacement by other amino acids resulted in at least 67% and 58% lower spot intensities. Amino acid positions ⁸T and ¹²P were less conserved, but exhibited a clear preference for the wild type amino acids; exchange by other amino acids resulted in 27% and 33% lower spot intensities.

All other N- and C-terminal amino acids were susceptible for exchange by any other amino acid and exhibited a variable character. The C-terminal preference for substitution by M or P was rather attributed to a structural effect than to a real effect on the antibody interaction.

Ab5.5_var1

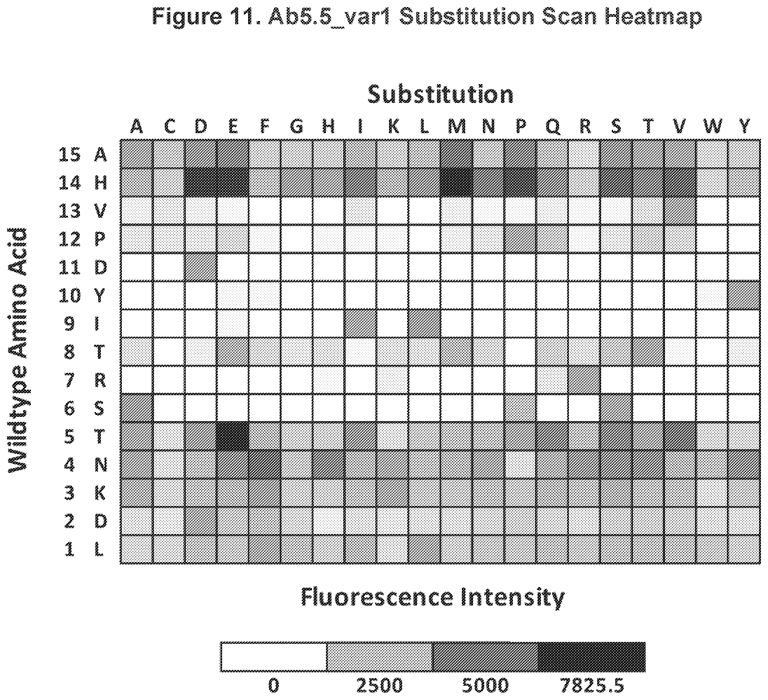

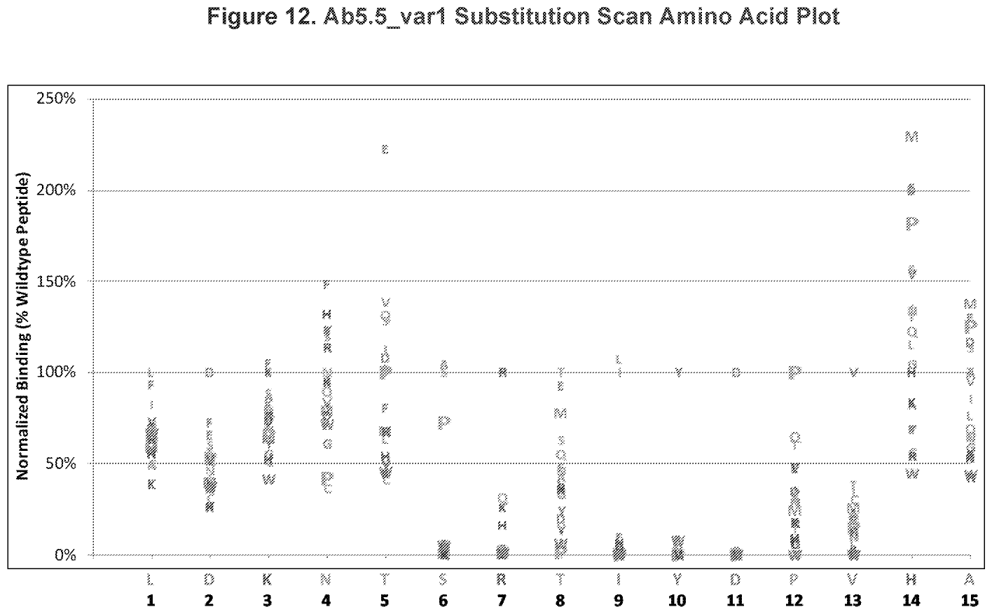

The heat map (FIG. 11) and the amino acid plot (FIG. 12) of Ab5.5_var1 assayed against the substitution scan of wild type peptide ¹LDKNTSRTIYDPVHA¹⁵ (SEQ ID NO:32) highlighted a conserved core motif ⁶SRTIYDPV¹³ (SEQ ID NO:22) framed by N- and C-terminal variable stretches ¹LDKNT⁵ (SEQ ID NO:34) and ¹⁴HA¹⁵. This finding was in accordance with the previous epitope mapping against human Ryk amino acids 134-227.

Very similar to Ab5.5, amino acid positions ¹⁰Y and ¹¹D were highly conserved, and exchange by other amino acids was not tolerated without a widely complete loss of antibody binding. Except for a high tolerance for a conserved exchange by L, the same high degree of sequence conservation was found for amino acid position ⁹I. Amino acid position ⁶S showed a similar tolerance for substitution by A and P, but was otherwise also highly conserved. Amino acid positions ⁷R and ¹³V were well conserved, since replacement by other amino acids resulted in at least 70% and 62% lower spot intensities. Amino acid positions ⁸T and ¹²P were less conserved, but exhibited a clear preference for the wild type amino acids; exchange by other amino acids resulted in 7.5% and 35% lower spot intensities.

All other N- and C-terminal amino acids were susceptible for exchange by any other amino acid and exhibited a variable character. The C-terminal preference for substitution by M, E, D or P was rather attributed to a structural effect than to a real effect on the antibody interaction.

### Example 5

### Ab5.5_var1 Inhibition of Canonical Wnt Signaling

### Methods

HEK 293 STF (ATCC® CRL-3249™) is a luciferase reporter cell line that has stable expression of 7× LEF/TCF and responds to canonical Wnt signaling by expressing the luciferase enzyme.

HEK 293 STF cells were seeded into 96 well plates at a density of 30,000 cells/well. After 36 hours, the cells were treated with Ab5.5_var1 at the indicated concentrations (0-500 μg/mL) in serum free minimum essential medium (MEM). After a 1-hour incubation with Ab5.5_var1, the medium was exchanged with MEM containing the indicated concentrations of Ab5.5_var1 and 250 ng/mL of human Wnt-3a recombinant protein (R&D Systems™ #5036WN010). After 24 hours of incubation with Ab5.5_var1 and Wnt-3a, luciferase was detected using the Steady-Glo™ Luciferase Assay System (Promega #E2510) and a Cytation™ 5 imaging system (BioTek).

A three-parameter log(inhibitor) vs. response nonlinear regression model was fit to the data with the bottom constrained to 0. In order to be used in the model, data from the group that received 0 μg/mL Ab5.5_var1 was plotted at 5 μg/mL (Log₁₀=0.699). Individual data points were plotted along with the non-linear regression fit (solid line) with 95% confidence intervals (dashed lines).

### Results

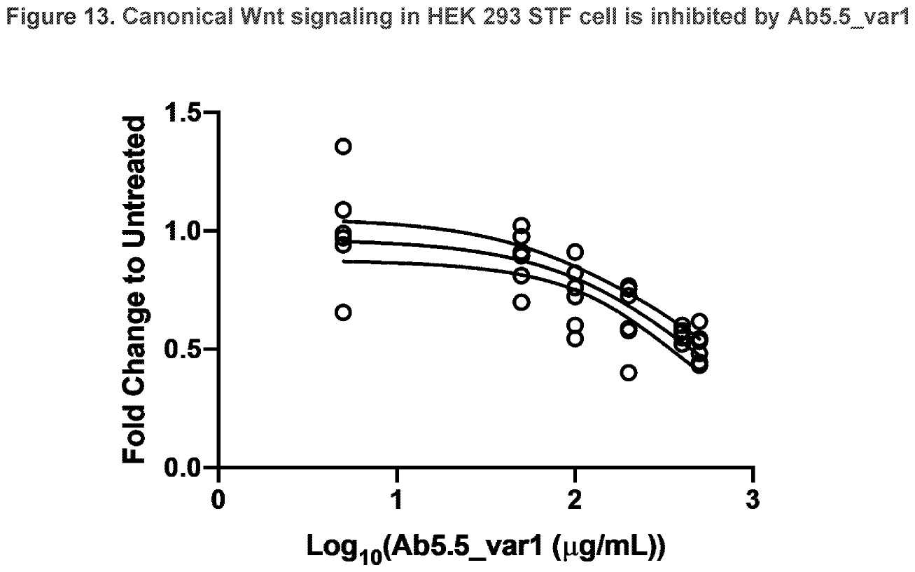

When HEK 293 STF cells were stimulated with Wnt-3a at 250 ng/mL, Ab5.5_var1 inhibited canonical Wnt signaling in a dose-dependent manner (FIG. 13). The dose response model (solid line with dashed lines at 95% confidence intervals) goodness of fit was R²=0.6168 and the IC50 of Ab5.5_var1 was calculated to be 484.5 μg/mL.

### Example 6

### RYK Expression in Cancer

### Methods

We analyzed the expression of Ryk mRNA in normal and tumor samples using the GEPIA2 web tool. GEPIA2 allows for researchers to perform gene expression and survival analysis using the data from the Cancer Genome Atlas (TCGA) and the Genotype-Tissue Expression (GTEx) projects that collectively have analyzed thousands of unique human samples.

Ryk gene expression analyses were performed for all of the available cancer types in the GEPIA2 program (Adrenocortical carcinoma, Bladder urothelial carcinoma, Breast invasive carcinoma, Cervical squamous cell carcinoma and endocervical adenocarcinoma, Cholangio carcinoma, Colon adenocarcinoma, Lymphoid neoplasm diffuse large B-cell lymphoma, Esophageal carcinoma, Glioblastoma multiforme, Head and neck squamous cell carcinoma, Kidney chromophobe, Kidney renal clear cell carcinoma, Kidney renal papillary cell carcinoma, Acute myeloid leukemia, Brain lower grade glioma, Liver hepatocellular carcinoma, Lung adenocarcinoma, Lung squamous cell carcinoma, Mesothelioma, Ovarian serous cystadenocarcinoma, Pancreatic adenocarcinoma, Pheochromocytoma and paraganglioma, Prostate adeonocarcinoma, Rectum adenocarcinoma, Sarcoma, Skin cutaneous melanoma, Stomach adenocarcinoma, Testicular germ cell tumors, Thyroid carcinoma, Thymoma, Uterine corpus endometrial carcinoma, Uterine carcinosarcoma, and Uveal melanoma).

Ryk mRNA expression values are represented as log₂(TPM+1). Tumors that had a mean fold change in Ryk mRNA expression greater than 2 and a one-way ANOVA p-value of less than 0.001 were considered to be significantly different than the control tissue. The number of tumor and normal samples included in each analysis are displayed beneath boxplots. White boxes indicate tumor samples, while grey boxes indicate normal samples.

Overall survival analysis based on Ryk mRNA expression was performed using samples from the top 25% and bottom 25% Ryk expression levels. The Log-rank test, cox proportional hazard ratio, and 95% confidence intervals were used to determine statistical significance. Survival curves show the low Ryk group in black and high Ryk group in light grey.

### Results

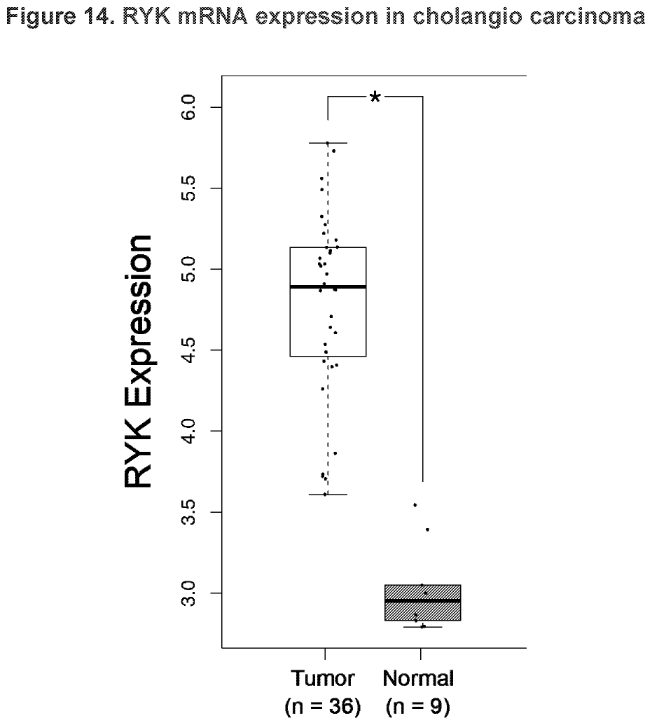

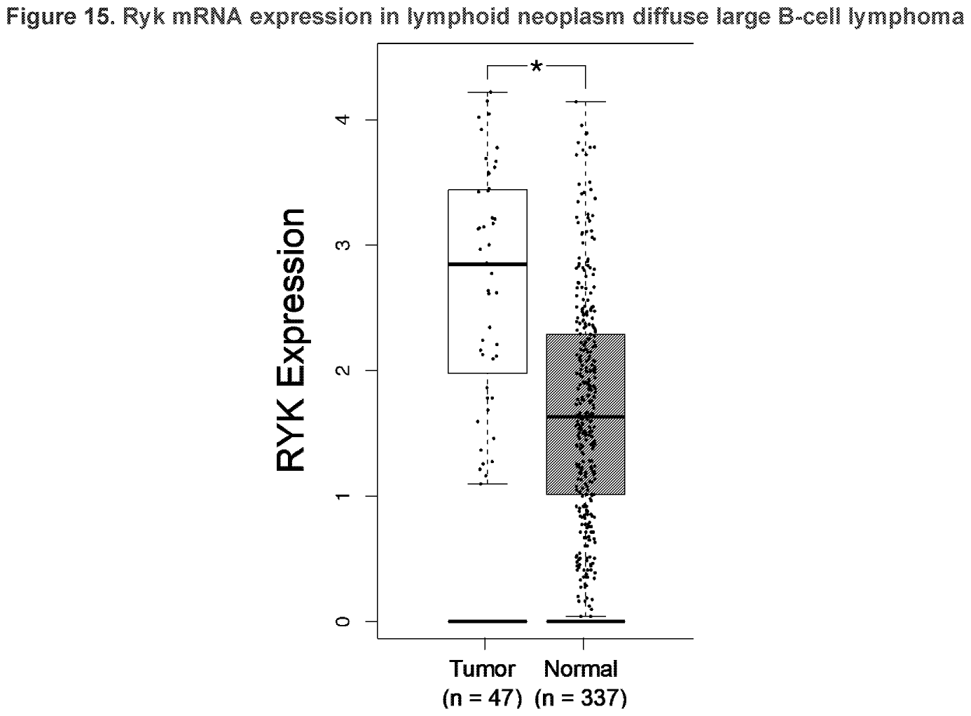

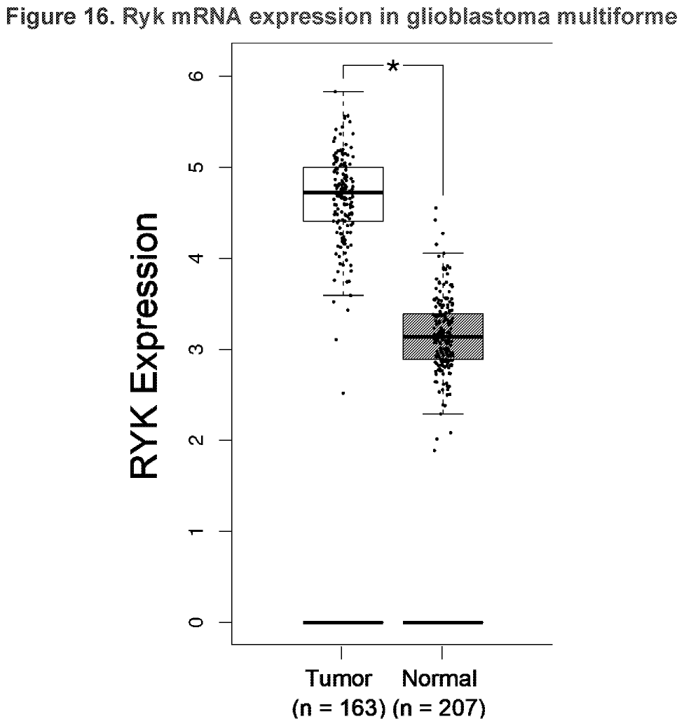

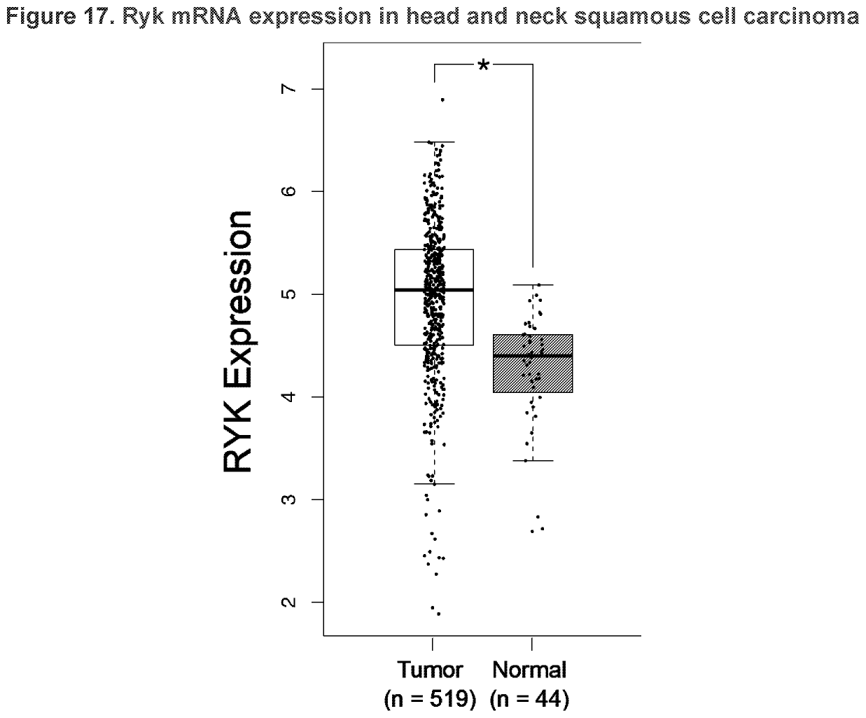

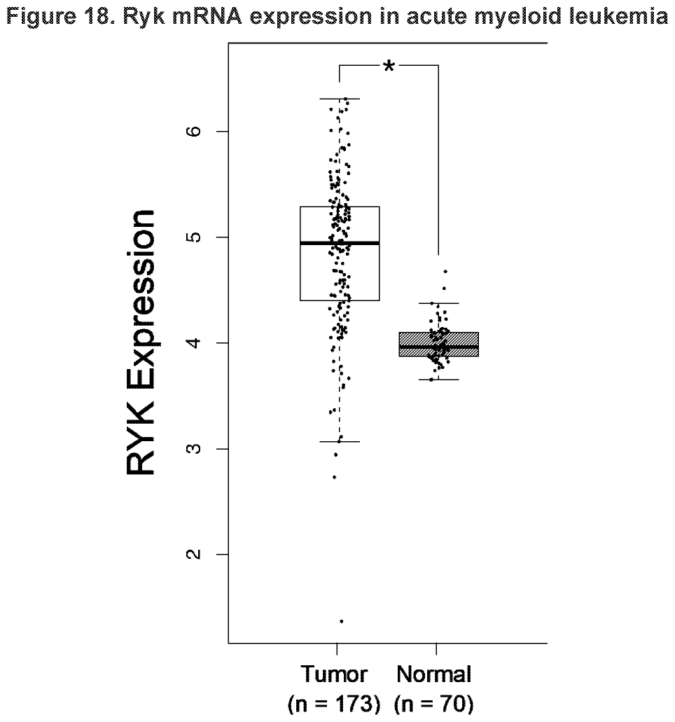

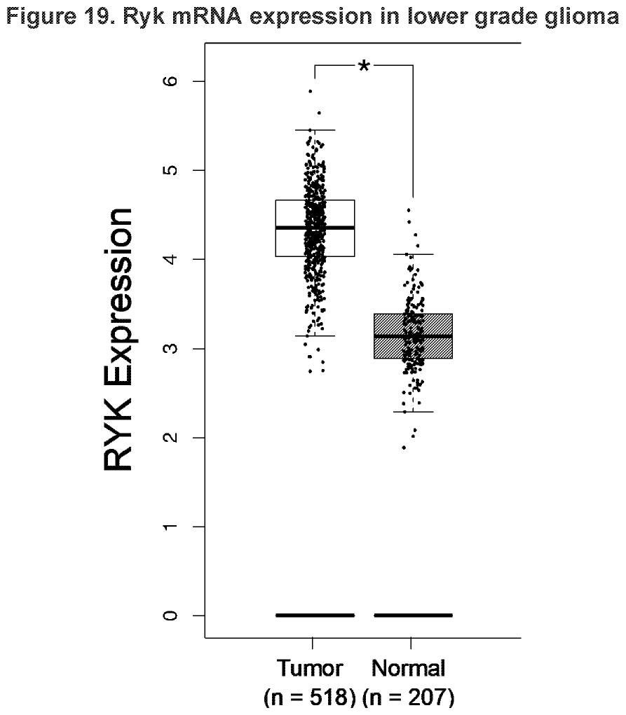

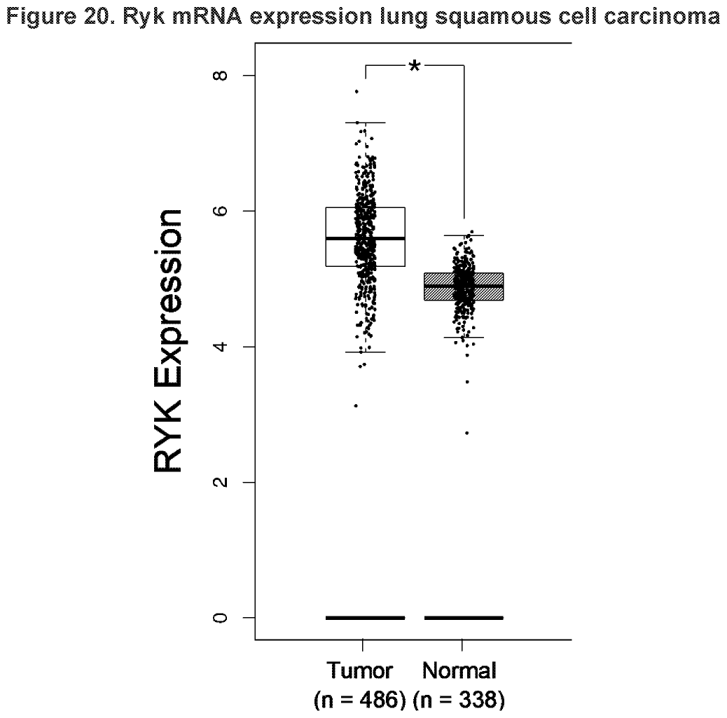

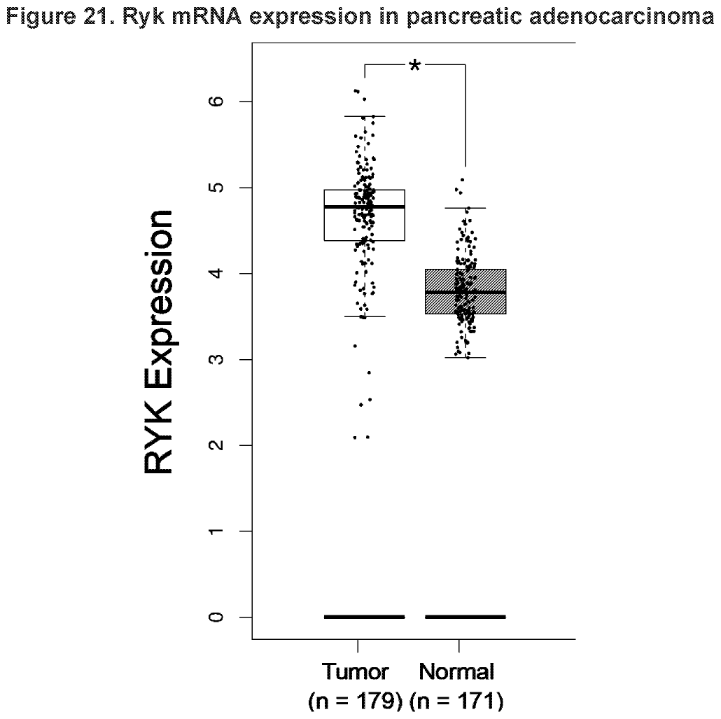

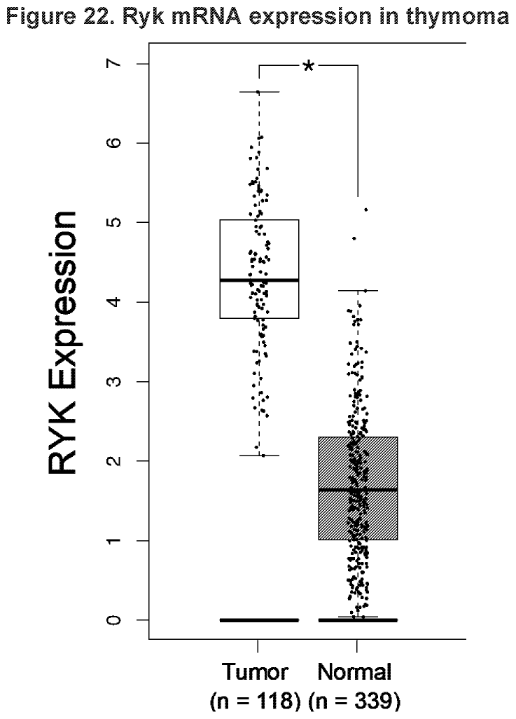

Ryk mRNA expression is significantly elevated in tumor samples from cholangio carcinoma (FIG. 14), lymphoid neoplasm diffuse large B-cell lymphoma (FIG. 15), glioblastoma multiforme (FIG. 16), head and neck squamous cell carcinoma (FIG. 17), acute myeloid leukemia (FIG. 18), lower grade glioma (FIG. 19), lung squamous cell carcinoma (FIG. 20), pancreatic adenocarcinoma (FIG. 21), and thymoma (FIG. 22).

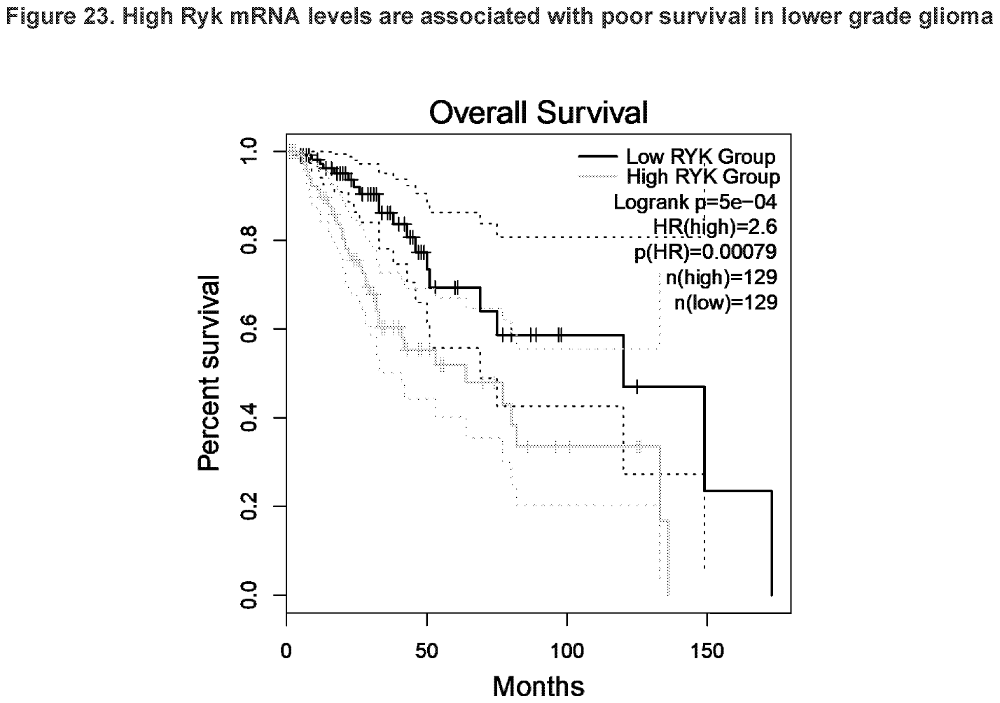

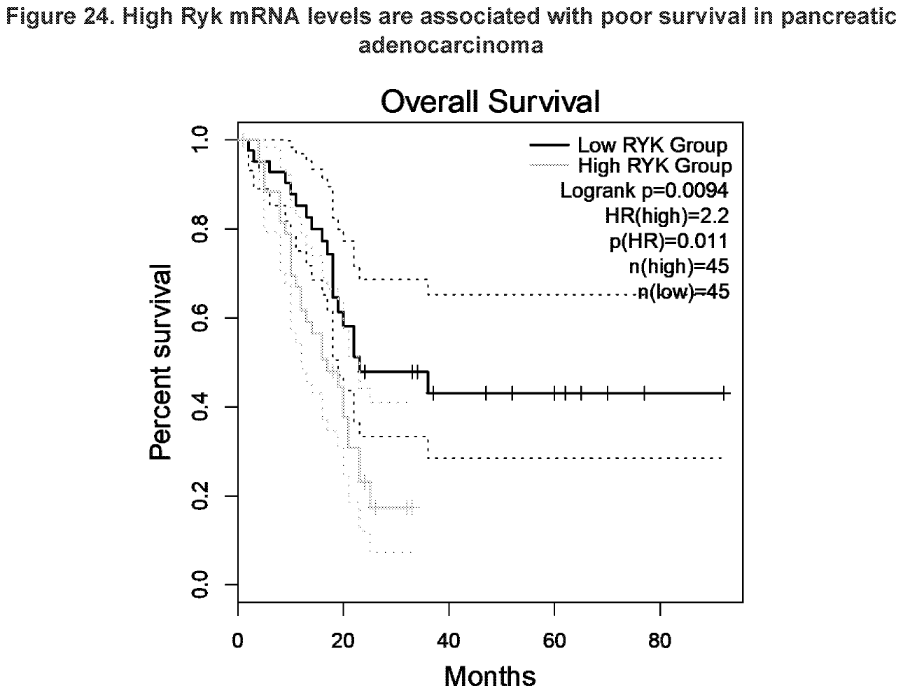

High levels of Ryk expression in tumor samples are significantly associated with poor survival in lower grade glioma (FIG. 23) and pancreatic adenocarcinoma (FIG. 24).

Reports in the scientific literature have confirmed that Ryk is involved in glioblastoma¹⁻⁴ and leukemia^{5,6}. Additional reports have documented that Ryk is involved in gastric cancer⁷, melanoma⁸, prostate cancer⁹, ovarian cancer¹⁰⁻¹², small cell lung cancer¹³, and atypical teratoid rhabdoid tumors¹⁴.

### Example 7

### Western Blot Validation of Ab5.5_Var1 by Using Immortal Human Cell Line Over-Expressed Mouse RYK and Human RYK

### Methods

### Plasmid and Cell Line

Plasmids were designed to express full length human RYK or full-length mouse RYK (see Macheda, Maria L., Willy W. Sun, Kumudhini Kugathasan, Benjamin M. Hogan, Neil I. Bower, Michael M. Halford, You Fang Zhang et al. “The Wnt receptor Ryk plays a role in mammalian planar cell polaiity signaling,” Journal of Biological Chemistry 287, no. 35 (2012): 29312-29323) (Macheda, Maria L. et al.). HEK 293 (ATCC, CRL-1573™ is a hypotriploid human cell line that commonly used for transfection and mammalian protein expression. Human embryonic kidney cells, HEK293, were cultured in DMEM (Gibco 11965118) medium supplemented with 10% Fetal Bovine Serum (FBS, Gibco A3840002) and 1× Penicillin-Streptomycin (Gibco, 15140-122), in an incubator with 37 ° C. and 5% CO₂.

### HEK293 Cell Transfection

HEK293 cell was seeded into 6-well plate with a density of 15,000 cell/well for 24 hours before transfection. During the transfection, 1 μg plasmid was mixed with 1 μl lipofectamine 3,000 (Invitrogen, L3000015) in 100 μl DMEM medium and placed in room temperature for 30 minutes, the mixture was then gradually added into each well of cells and then cultured for 4 hours in the 37° C. incubator. After the incubation, the medium of cell culture plate was replaced by DMEM containing 10% FBS and 1× Penicillin-Streptomycin and the cell culture plate was then placed in the incubator for another 24 hours.

### Lysing Cells

To lyse mammalian cell, use 200 μl RIPA lysis buffer (Thermo Scientific, J63324.EQE) with 1× Protease Inhibitor Cocktail (Thermo Scientific™ Halt™ Phosphatase Inhibitor Cocktail PI78420) per well of 6-well cell culture plate. Lyse for 10 minutes on ice. Collect lysate by spinning at 10,000 g for 10 minutes.

### Sample Preparation and SDS-PAGE

The protein concentration of each sample was determined using the 660nm protein assay with BSA standards. 4× Laemmli Sample Buffer (BIO-RAD Catalog #161-0747) was prepared by adding 0.1 mL 2-Mercaptoethanol (#1610710) for every 0.9 mL of 4× sample buffer. 4× Laemmli Sample Buffer was then added to each sample in a 1:3 ratio. Samples were placed in room temperature for 10 minutes. 2 μL of molecular weight marker (Precision Plus Protein™ All Blue Prestained Protein Standards #1610373) were used for each gel. 30 μg of each sample were loaded into the gel. Gel was run at 180V for 60 minutes. Use the Trans-BlotR Turbo™ Mini Nitrocellulose Transfer Packs (#1704158) in the Trans-BlotR Turbo™ Transfer System using the Mixed-MW protocol.

### Western Blot

Following transfer, membranes were dried and labeled using a black LI-COR pen. Membranes were reactivated with water, then blocked for 1 hour at room temperature using Odyssey PBS blocking buffer (Cat #) diluted 1:1 with 1× PBS. Following blocking, membrane was incubated overnight at 4° C. with Ab5.5_Var1 at a concentration of 1 μg/mL. After primary antibody incubation, membranes were rinsed 3× for 5 minutes with PBS-T and then incubated with anti-human (LiCOR) antibodies at a 1:15,000 dilution for 1 hour at room temperature. Membranes were then washed 3× for 5 minutes in PBS-T and the last PBS-T washed was exchanged with water. Membranes were then dried and scanned with a LI-COR CLx (Auto, 169 micron, medium quality, 0 offset).

### Results

Ab5.5 was raised against a 93 amino acid sequence of the mouse Ryk protein that contains two different amino acids than the human Ryk protein. To be an effective therapeutic protein in humans, it is essential that the humanized Ab5.5 variants recognize the human Ryk sequence. In order to confirm that the Ab5.5_Var1 recognize the human protein, we performed a series of Western blot experiments using the full-length human-RYK and full-length mouse-RYK expression protein as previously described (Macheda, Maria L. et al.).

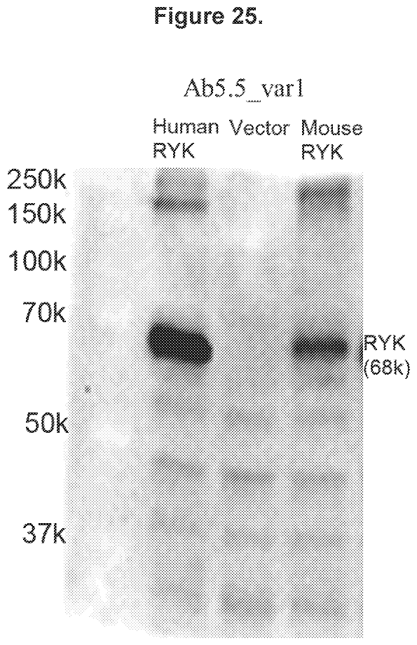

A representative result from the Western Blot using Ab5.5_Var1 and full-length human-RYK, full-length mouse RYK as well as empty vector as control is shown in FIG. 25. This data demonstrates that Ab5.5_Var1 strongly recognizes human cell expressed human Ryk protein.

### Example 8

### Ab5.5_var1 Blocks Non-Canonical Wnt Signaling, Wnt5a, Induce Migration in Human Neuroblastoma Cell Line, SK-N-SH

### Methods

SK-N-SH cell line (ATCC HTB-11) is a human neuroblastoma cell line that commonly used for multiple cell-based laboratory assays. SK-N-SH cell were cultured in ATCC-formulated Eagle's Minimum Essential Medium, Catalog No. 30-2003 supplemented with 10% Fetal Bovine Serum (FBS, Gibco A3840002) and 1× Penicillin-Streptomycin (Gibco, 15140-122), cultured in an incubator with 37° C. and 5% CO₂.

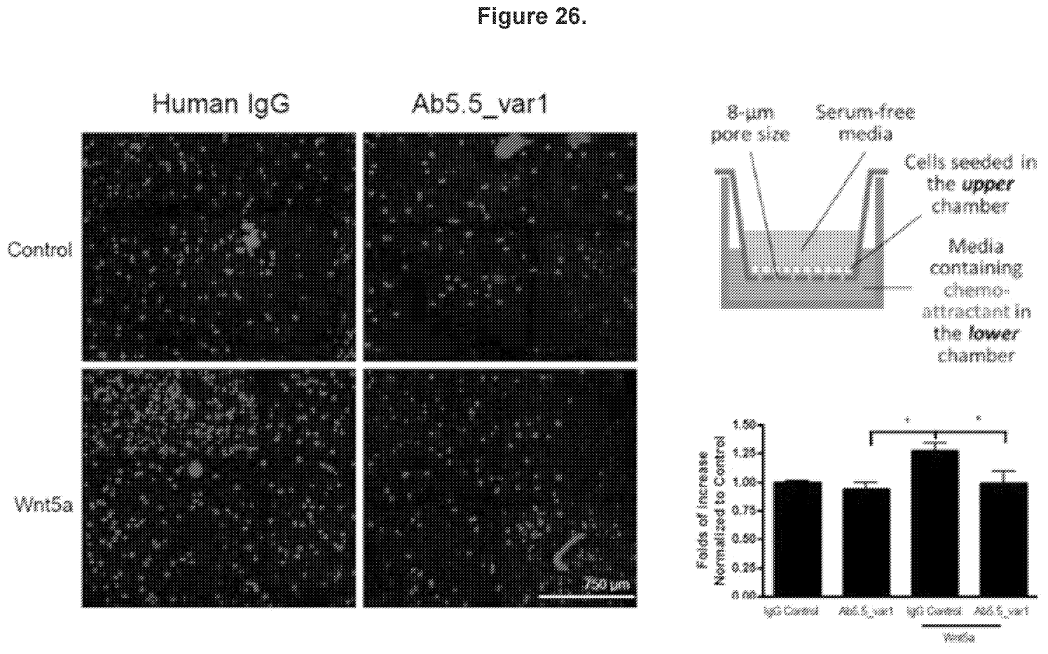

As shown in FIG. 26, SK-N-SH was seeded into 24 well transwell plate that contained an insert with 8 um core size (Falcon, 353097), with a density of 8000 cell per well. Then add Ab5.5 Var1 or human IgG control antibody (Invitrogen, Human IgG Isotype Control, 02-7102) at a concentration of 25 μg/mL to the bottom of each well and placed in 37° C. incubator for 1 hour. The transwell plate was then take out and added Wnt-5a recombinant Protein (R&D Systems, 645WN010) at a concentration of 300 ng/mL to the bottom of each well. The plate was placed in the incubator for 24 hours.

After incubation, 24 well transwell plate was placed on room temperature and the insert was washed 3× with 1× PBS. Then the insert was fixed with 4% Paraformaldehyde at room temperature for 20 minutes. After fixation, insert was stained with Hoechst 33342 Solution (Thermo Scientific, 62249) at a concentration of 1 μg/ml.

Images was captured by using Cytation 5 Cell Imaging Multi-Mode Reader (BioTek) under 4× objective lens by using DAPI observing channel. Cell number per insert (well) was automatically counted by the program of Cytation 5 viewer.

For quantification, cell numbers in each well from all replicated experiments was statistically analyzed by Student's t test. *, p<0.05.

### Results

As shown in FIG. 26, when the SK-N-SH cell migration was stimulated with Wnt5a at 300 ng/mL, Ab5.5_Var1 inhibited this non-canonical Wnt signaling and its mediated cell migration. The inhibition caused by Ab5.5_Var1 is specific to Wnt5a induced non-canonical Wnt signaling and its mediated cell migration.

### Example 9

### Ab5.5_var1 Mediates αHFc-CL-PNU Antibody Conducted Cytotoxicity in Human T Cell Lymphoblastic Cell Line, MOLT4

### Methods

MOLT4 (ATCC, CRL-1582) is a human T lymphoblast cell collected from Acute lymphoblastic leukemia (ALL) patient. MOLT4 cell were culture with ATCC-formulated RPMI-1640 Medium (ATCC 30-2001) supplemented with 10% Fetal Bovine Serum (FBS, Gibco A3840002) and 1× Penicillin-Streptomycin (Gibco, 15140-122), cultured in an incubator with 37° C. and 5% CO₂.

αHFc-CL-PNU is “IgGs Anti-Human IgG Fc-PNU159682 Antibody with Cleavable Linker” (Moradec, AH-102PN-50), this is a conjugate of a human IgG with a cytotoxin PNU159682.

MOLT4 cell were seeded into 96 well cell culture plate with a density of 10,000 cell per well. After 24 hours incubation, cells were treated with Ab5.5_Var1 along, mixture of Ab5.5_Var1 with αHFc-CL-PNU or a mixture of human IgG with αHFc-CL-PNU. The mixture of treatment and cells were then incubated for another 72 hours.

After incubation, cells were treated with 10% Alamar-blue (Invitrogen, DAL1025) and placed in 37° C. incubator for 1 hour. After incubation, plate was read by using Cytation 5 Cell Imaging Multi-Mode Reader (BioTek).

For quantification, cell numbers in each well from all replicated experiments was statistically analyzed by Student's t test. *, p<0.05.

### Results

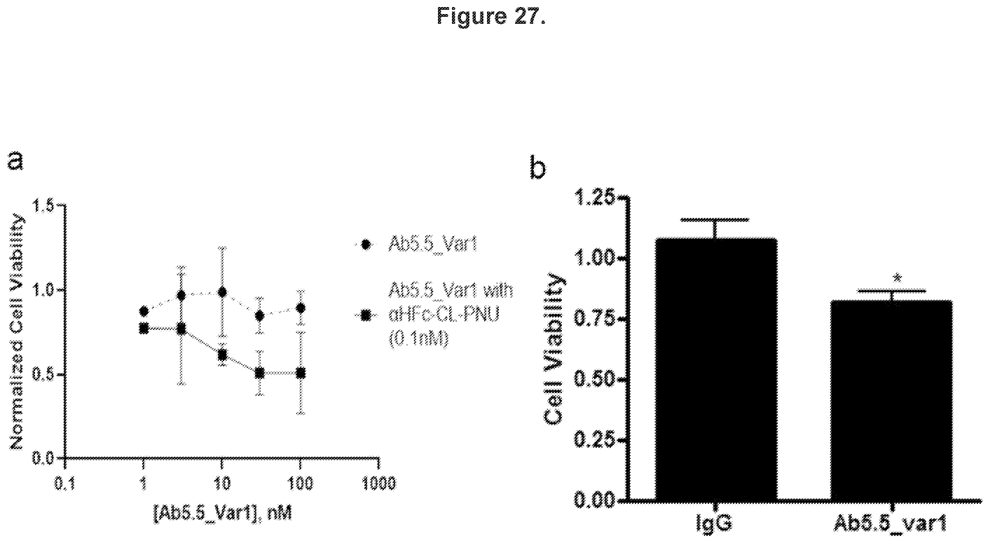

As shown in FIG. 27a, when treated together with 0.1 nM αHFc-CL-PNU, Ab5.5_Var1 reduced cell viability in a dose-dependent manner, EC50 is 22.39 nM.

As shown in FIG. 27b, when treated together with αHFc-CL-PNU, Ab5.5_Var1 could reduce 24% of cell viability compared with control IgG.

### Example 10

### Ab5.5_var1 Blocks Wnt5a Induced RhoA Activation

### Methods

### Plasmid and Cell Line

Plasmids were designed to express full length human RYK and full length human Frizzled3 (see Onishi, Keisuke, Beth Shafer, Charles Lo, Fadel Tissir, Andre M. Goffinet, and Yimin Zou. “Antagonistic functions of Dishevelleds regulate Frizzled3 endocytosis via filopodia tips in Wnt-mediated growth cone guidance.” Journal of Neuroscience 33, no. 49 (2013): 19071-19085).

HEK 293 (ATCC, CRL-1573™) is a hypotriploid human cell line that commonly used for transfection and mammalian protein expression. Human embryonic kidney cells, HEK293, were cultured in DMEM (Gibco 11965118) medium supplemented with 10% Fetal Bovine Serum (FBS, Gibco A3840002) and 1× Penicillin-Streptomycin (Gibco, 15140-122), in an incubator with 37° C. and 5% CO₂.

### HEK293 Cell Transfection

HEK293 cell was seeded into 6-well plate with a density of 15,000 cell/well for 24 hours before transfection. During the transfection, 11 μg total plasmid was mixed with 1 μl lipofectamine 3000 (Invitrogen, L3000015) in 100 μl DMEM medium and placed in room temperature for 30 minutes, the mixture was then gradually added into each well of cells and then cultured for 4 hours in the 37° C. incubator. After the incubation, the medium of cell culture plate was replaced by DMEM containing 10% FBS and 1× Penicillin-Streptomycin and the cell culture plate was then placed in the incubator for another 24 hours.

### RhoA Activation Assay

Transfected cell starved by using DMEM medium that do not contain FBS 24 hours prior to stimulation. After starvation, cells were treated with either 200 ng/mL human recombinant Wnt5a or 200 ng/mL Wnt5a together with 20 μg/ml Ab5.5_Var1. Cells were placed in 37° C. for 30 minutes.

After stimulation, active form of RhoA was detected by using RhoA G-LISA Activation Assay kit (Cytoskeleton, BK124) and following manufacturer's instruction.

Quantification was performed by using Student's t test for data collected from all replicated experiments.

### Results

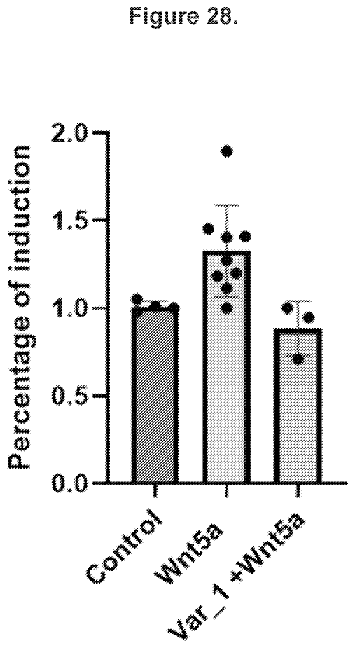

As shown in FIG. 28, Wnt5a stimulation could increase the active form of RhoA by 32.6%. Ab5.5_Var1 treatment with Wnt5a together could block this stimulation.

Sequences

Various sequences are listed in the sequence table below.

SEQUENCE TABLE

| SEQ ID NO | Sequence (5′-3′ or N-C) | Description |
|---|---|---|
| SEQ ID NO: 1 | RANRLVE | CDR_L2 |
| SEQ ID NO: 2 | STGGGGTY | CDR_H2 |
| SEQ ID NO: 3 | HGDSGDY | CDR_H3_1 |
| SEQ ID NO: 4 | HGDQGDY | CDR_H3_2 |
| SEQ ID NO: 5 | KASQDINSYLS | CDR_L1 |
| SEQ ID NO: 6 | LQYDEFPLT | CDR_L3 |
| SEQ ID NO: 7 | GFTFSSY | CDR_H1 |
| SEQ ID NO: 8 | HGDNGDY | CDR_H3_3 |
| SEQ ID NO: 9 | DIKMTQSPSSMYASLGERVTITCKASQDINSYLSWI | Ab5.5_VL |
| | QQKPGKSPKTLIYRANRLVDGVPSRFSGSGSGQDY | |
| | SLTISSLEYEDMGIYYCLQYDEFPLTFGAGTKLELK | |
| SEQ ID NO: 10 | EVKLVESGGDLVQPGGSLKLSCAASGFTFSSYTMS | Ab5.5_VH |
| | WIRQTPEKRLEWVAYISNGGGGTYYPDTVKGRFTI | |
| | SRDNAKNTLYLQMNSLKSEDTAMYYCTRHGDNG | |
| | DYWGHGSTLTVSS | |
| SEQ ID NO: 11 | DIQMTQSPSSLSASVGDRVTITCKASQDINSYLSWI | Ab5.5_VL_1 |
| | QQKPGKAPKTLIYRANRLVDGVPSRFSGSGSGTDY | |
| | TLTISSLQPEDFATYYCLQYDEFPLTFGAGTKVEIK | |
| SEQ ID NO: 12 | DIQMTQSPSSLSASVGDRATITCKASQDINSYLSWI | Ab5.5_VL_2 |
| | QQKPGKAPKTLIYRANRLVDGVPSRFSGSGSGTDY | |
| | TLTISSLQPEDFATYYCLQYDEFPLTFGGGTKVEIK | |
| SEQ ID NO: 13 | DIQMTQSPSSLSASVGDRATITCKASQDINSYLSWI | Ab5.5_VL_3 |
| | QQKPGKAPKTLIYRANRLVEGVPSRFSGSGSGTDY | |
| | TLTISSLQPEDFATYYCLQYDEFPLTFGGGTKVEIK | |
| SEQ ID NO: 14 | EVQLVESGGGLVQPGGSLRLSCAASGFTFSSYTMS | Ab5.5_VH_1 |
| | WIRQAPGKGLEWVAYISNGGGGTYYPDSVKGRFTI | |
| | SRDNAKNTLYLQMNSLRAEDTAVYYCTRHGDNG | |
| | DYWGHGSLVTVSS | |
| SEQ ID NO: 15 | EVQLVESGGGLVQPGGSLRLSCAASGFTFSSYTMS | Ab5.5_VH_2 |
| | WIRQAPGKGLEWVAYISNGGGGTYYPDSVKGRFTI | |
| | SRDNAKNSLYLQMNSLRAEDTAVYYCTRHGDNG | |
| | DYWGQGSLVTVSS | |
| SEQ ID NO: 16 | EVQLVESGGGLVQPGGSLRLSCAASGFTFSSYTMS | Ab5.5_VH_3 |
| | WIRQAPGKGLEWVAYISTGGGGTYYPDSVKGRFTI | |
| | SRDNAKNSLYLQMNSLRAEDTAVYYCTRHGDSGD | |
| | YWGHGSLVTVSS | |
| SEQ ID NO: 17 | EVQLVESGGGLVQPGGSLRLSCAASGFTFSSYTMS | Ab5.5_VH_4 |
| | WIRQAPGKGLEWVAYISTGGGGTYYPDSVKGRFTI | |
| | SRDNAKNSLYLQMNSLRAEDTAVYYCTRHGDQG | |
| | DYWGHGSLVTVSS | |
| SEQ ID NO: 18 | EVQLVESGGGLVQPGGSLRLSCAASGFTFSSYTMS | Ab5.5_VH_5 |
| | WIRQAPGKGLEWAAYISTGGGGTYYVDSVKGRFT | |
| | ISRDNAKNSLYLQMNSLRAEDTAVYYCTRHGDNG | |
| | DYWGHGSLVTVSS | |
| SEQ ID NO: 19 | SRTIYDPV | Homo sapiens Ryk(211-218) |
| SEQ ID NO: 20 | ARTIYDPV | Ryk artificial peptide 1 |
| SEQ ID NO: 21 | PRTIYDPV | Ryk artificial peptide 2 |
| SEQ ID NO: 22 | SRTLYDPV | Ryk artificial peptide 3 |
| SEQ ID NO: 23 | SRXIYDPV (X being a natural amino acid | Ryk artificial peptide 4 |
| | that is not T) | |
| SEQ ID NO: 24 | MRAGRGGVPGSGGLRAPPPPLLLLLLAMLPAAAPR | Mus musculus Ryk |
| | SPALAAAPAGPSVSLYLSEDEVRRLLGLDAELYYV | |
| | RNDLISHYALSFNLLVPSETNFLHFTWHAKSKVEY | |
| | KLGFQVDNFVAMGMPQVNISAQGEVPRTLSVFRV | |
| | ELSCTGKVDSEVMILMQLNLTVNSSKNFTVLNFKR | |
| | RKMCYKKLEEVKTSALDKNTSRTIYDPVHAAPTTS | |
| | TRVFYISVGVCCAVIFLVAIILAVLHLHSMKRIELD | |
| | DSISASSSSQGLSQPSTQTTQYLRADTPNNATPITSS | |
| | SGYPTLRIEKNDLRSVTLLEAKAKVKDIAISRERITL | |
| | KDVLQEGTFGRIFHGILVDEKDPNKEKQTFVKTVK | |
| | DQASEVQVTMMLTESCKLRGLHHRNLLPITHVCIE | |
| | EGEKPMVVLPYMNWGNLKLFLRQCKLVEANNPQ | |
| | AISQQDLVHMAIQIACGMSYLARREVIHRDLAARN | |
| | CVIDDTLQVKITDNALSRDLFPMDYHCLGDNENRP | |
| | VRWMALESLVNNEFSSASDVWAFGVTLWELMTL | |
| | GQTPYVDIDPFEMAAYLKDGYRIAQPINCPDELFA | |
| | VMACCWALDPEERPKFQQLVQCLTEFHAALGAYV | |
| SEQ ID NO: 25 | MRGAARLGRPGRSCLPGARGLRAPPPPPLLLLLAL | Homo sapiens Ryk |
| | LPLLPAPGAAAAPAPRPPELQSASAGPSVSLYLSED | |
| | EVRRLIGLDAELYYVRNDLISHYALSFSLLVPSETN | |
| | FLHFTWHAKSKVEYKLGFQVDNVLAMDMPQVNIS | |
| | VQGEVPRTLSVFRVELSCTGKVDSEVMILMQLNLT | |
| | VNSSKNFTVLNFKRRKMCYKKLEEVKTSALDKNT | |
| | SRTIYDPVHAAPTTSTRVFYISVGVCCAVIFLVAIIL | |
| | AVLHLHSMKRIELDDSISASSSSQGLSQPSTQTTQY | |
| | LRADTPNNATPITSYPTLRIEKNDLRSVTLLEAKGK | |
| | VKDIAISRERITLKDVLQEGTFGRIFHGILIDEKDPN | |
| | KEKQAFVKTVKDQASEIQVTMMLTESCKLRGLHH | |
| | RNLLPITHVCIEEGEKPMVILPYMNWGNLKLFLRQ | |
| | CKLVEANNPQAISQQDLVHMAIQIACGMSYLARRE | |
| | VIHKDLAARNCVIDDTLQVKITDNALSRDLFPMDY | |
| | HCLGDNENRPVRWMALESLVNNEFSSASDVWAFG | |
| | VTLWELMTLGQTPYVDIDPFEMAAYLKDGYRIAQ | |
| | PINCPDELFAVMACCWALDPEERPKFQQLVQCLTE | |
| | FHAALGAYV | |
| SEQ ID NO: 26 | DMPQVNISVQGEVPRTLSVFRVELSCTGKVDSEVM | Homo sapiens Ryk(134-227) |
| | ILMQLNLTVNSSKNFTVLNFKRRKMCYKKLEEVK | |
| | TSALDKNTSRTIYDPVHAAPTTSTR | |
| SEQ ID NO: 27 | GSGSGSG | Neutral peptide linker |
| SEQ ID NO: 28 | TSRTIYDPV | Homo sapiens Ryk(210-218) |
| SEQ ID NO: 29 | SSKNFTVLNFKRRK | Homo sapiens Ryk(179-192) |
| SEQ ID NO: 30 | TVLNFKRRKMCYKK | Homo sapiens Ryk(184-197) |
| SEQ ID NO: 31 | RRKMCYKKLEEVK | Homo sapiens Ryk(190-202) |
| SEQ ID NO: 32 | LDKNTSRTIYDPVHA | Homo sapiens Ryk(206-220) |
| SEQ ID NO: 33 | YPYDVPDYAG | HA tag peptide |
| SEQ ID NO: 34 | LDKNT | Homo sapiens Ryk(206-210) |
| SEQ ID NO: 35 | NSSKNFTVLNFKRRK | Homo sapiens Ryk(178-192) |
| SEQ ID NO: 36 | VLNFKRRKMCYKK | Homo sapiens Ryk(185-197) |

References

References cited in this Example are listed below.
1. Habu M, Koyama H, Kishida M, Kamino M, Iijima M, Fuchigami T, Tokimura H, Ueda M, Tokudome M, Koriyama C, Hirano H, Arita K, Kishida S. Ryk is essential for Wnt-5a-dependent invasiveness in human glioma. J Biochem. 2014 Jul. 1;156(1):29-38.
2. Hirano H, Yonezawa H, Yunoue S, Habu M, Uchida H, Yoshioka T, Kishida S, Kishida M, Oyoshi T, Fujio S, Sugata S, Yamahata H, Hanaya R, Arita K. Immunoreactivity of Wnt5a, Fzd2, Fzd6, and Ryk in glioblastoma: evaluative methodology for DAB chromogenic immunostaining. Brain Tumor Pathol. 2014 Apr. 1;31(2):85-93.
3. Kim Y, Hong M, Do I-G, Ha SY, Lee D, Suh Y-L. Wnt5a, Ryk and Ror2 expression in glioblastoma subgroups. Pathol Res Pract. 2015 December;211(12):963-972. PMID: 26596412
4. Adamo A, Fiore D, De Martino F, Roscigno G, Affinito A, Donnarumma E, Puoti I, Ricci Vitiani L, Pallini R, Quintavalle C, Condorelli G. RYK promotes the stemness of glioblastoma cells via the WNT/ f3-catenin pathway. Oncotarget. 2017 Feb. 21;8(8):13476-13487. PMCID: PMC5355113
5. Alvarez-Zavala M, Riveros-Magaña A R, García-Castro B, Barrera-Chairez E, Rubio-Jurado B, Garcés-Ruiz O M, Ramos-Solano M, Aguilar-Lemarroy A, Jave-Suarez L F. WNT receptors profile expression in mature blood cells and immature leukemic cells: RYK emerges as a hallmark receptor of acute leukemia. European Journal of Haematology. 2016;97(2):155-165.
6. Micci F, Panagopoulos I, Haugom L, Andersen H K, Tjønnfjord G E, Beiske K, Heim S. t(3;21)(q22;q22) leading to truncation of the RYK gene in atypical chronic myeloid leukemia. Cancer Letters. 2009 May 18;277(2):205-211.
7. Fu Y, Chen Y, Huang J, Cai Z, Wang Y. R Y K, a receptor of noncanonical Wnt ligand Wnt5a, is positively correlated with gastric cancer tumorigenesis and potential of liver metastasis. American Journal of Physiology-Gastrointestinal and Liver Physiology. American Physiological Society; 2019 Dec. 23;318(2):G352—G360.
8. Anastas J N, Kulikauskas R M, Tamir T, Rizos H, Long G V, Euw E M von, Yang P-T, Chen H-W, Haydu L, Toroni R A, Lucero O M, Chien A J, Moon R T. WNTSA enhances resistance of melanoma cells to targeted BRAF inhibitors. J Clin Invest. American Society for Clinical Investigation; 2014 Jul. 1;124(7):2877-2890. PMID: 0
9. Thiele S, Zimmer A, Göbel A, Rachner T D, Rother S, Fuessel S, Froehner M, Wirth M P, Muders M R, Baretton G B, Jakob F, Rauner M, Hofbauer L C. Role of WNTSA receptors FZDS and RYK in prostate cancer cells. Oncotarget. Impact Journals; 2018 May 25;9(43):27293-27304.
10. Wang X C, Katso R, Butler R, Hanby A M, Poulsom R, Jones T, Sheer D, Ganesan T S. H-RYK, an unusual receptor kinase: isolation and analysis of expression in ovarian cancer. Mol Med. 1996 March;2(2):189-203. PMCID: PMC2230112
11. Katso R M T, Manek S, Biddolph S, Whittaker R, Charnock M F L, Wells M, Ganesan T S. Overexpression of H-Ryk in Mouse Fibroblasts Confers Transforming Ability in Vitro and in Vivo: Correlation with Up-Regulation in Epithelial Ovarian Cancer. Cancer Res. American Association for Cancer Research; 1999 May 15;59(10):2265-2270. PMID: 10344726
12. Katso R M T, Manek S, Ganjavi H, Biddolph S, Charnock M F L, Bradburn M, Wells M, Ganesan T S. Overexpression of H-Ryk in Epithelial Ovarian Cancer: Prognostic Significance of Receptor Expression. Clin Cancer Res. American Association for Cancer Research; 2000 Aug. 1;6(8):3271-3281. PMID: 10955813
13. Hamilton G, Rath B, Klameth L, Hochmair M. Receptor tyrosine kinase expression of circulating tumor cells in small cell lung cancer. Oncoscience. 2015;2(7):629-634. PMCID: PMC4549360
14. Chakravadhanula M, Hampton C N, Chodavadia P, Ozols V, Zhou L, Catchpoole D, Xu J, Erdreich-Epstein A, Bhardwaj R D. Wnt pathway in atypical teratoid rhabdoid tumors. Neuro Oncol. Oxford Academic; 2015 Apr. 1;17(4):526-535.

### Additional References

Additional selected references cited herein are listed below.

### References

Al-Lazikani B, Lesk A M, Chothia C. (1997). Standard conformations for the canonical structures of immunoglobulins. J. Mol. Biol. 273, 927-948.
Almagro J C, Beavers M P, Hernandez-Guzman F, Maier J, Shaulsky J, Butenhof K, Labute P, Thorsteinson N, Kelly K, Teplyakov A, Luo J, Sweet R, Gilliland G L. (2011). Antibody modeling assessment. Proteins 79, 3050-3066.
Baker M P, Jones T D. (2007). Identification and removal of immunogenicity in therapeutic proteins. Curr. Opin. Drug Discov. Devel. 10, 219-227.
Berdoz J, Gorski J, Termijtelen A M, Dayer J M, Irle C, Schendel D, Mach B. (1987). Constitutive and induced expression of the individual HLA-DR beta and alpha chain loci in different cell types. J. Immunol. 139, 1336-1341.
Chamberlain P. (2002). Immunogenicity of therapeutic proteins Part 1: Causes and clinical manifestations of immunogenicity. The Regulatory Review 5, 4-9.
Chothia C, Lesk A M. (1987). Canonical structures for the hypervariable regions of immunoglobulins. J. Mol. Biol. 196, 901-917.
Desmet J, Spriet J, Lasters I. (2002). Fast and accurate side-chain topology and energy refinement (FASTER) as a new method for protein structure optimisation. Proteins 48, 31-43.
Ewert S, Huber T, Honegger A, Pluckthun A. (2003). Biophysical properties of human antibody variable domains. J. Mol. Biol. 325, 531-553.
Foote J, Winter G. (1992). Antibody framework residues affecting the conformation of the hypervariable loops. J. Mol. Biol. 224, 487-499.
Gansbacher B, Zier K S. (1988). Regulation of HLA-DR, DP, and DQ expression in activated T cells. Cell Immunol. 117, 22-34.
Harding F A, Stickler M M, Razo J, DuBridge R B. (2010). The immunogenicity of humanised and fully human antibodies: residual immunogenicity resides in the CDR regions. MAbs. 2, 256-265.
Honegger A, Pluckthun A. (2001). Yet another numbering scheme for immunoglobulin variable domains: an automatic modeling and analysis tool. J. Mol. Biol. 309, 657-670.
Honegger A, Malebranche A D, Rothlisberger D, Pluckthun A. (2009). The influence of the framework core residues on the biophysical properties of immunoglobulin heavy chain variable domains. Protein Eng. Des. Se.l 22, 121-134.
Jones P T, Dear P H, Foote J, Neuberger M S, Winter G. (1986). Replacing the complementarity-determining regions in a human antibody with those from a mouse. Nature 321, 522-525.
Kabat E A, Wu T T, Perry H, Gottesman K, Foeller C. (1991). Sequences of Proteins of Immunological Interest, Fifth Edition. NIH Publication.
Kirschmann D A, Duffin K L, Smith C E, Welply J K, Howard S C, Schwartz B D, Woulfe S L. (1995). Naturally processed peptides from rheumatoid arthritis associated and non-associated HLA-DR alleles. J. Immunol. 155, 5655-5662.
Laupeze, B, Fardel O, Onno M, Bertho N, Drenou B., Fauchet R, Amiot L. (1999). Differential expression of major histocompatibility complex class Ia, Ib, and II molecules on monocytes-derived dendritic and macrophagic cells. Hum. Immunol. 60, 591-597.
North B, Lehmann A, Dunbrack R L Jr. (2011). A new clustering of antibody CDR loop conformations. J. Mol. Biol. 406, 228-256.
Stas P, Pletinckx J, Gansemans Y, Lasters I. (2009). Immunogenicity assessment of antibody therapeutics. Cambridge University Press, Cambridge.
Stunz L L, Karr R W, Anderson R A. (1989). HLA-DRB1 and -DRB4 genes are differentially regulated at the transcriptional level. J. Immunol. 143, 3081-3086.
Van W I, Gansemans Y, Parren P W, Stas P, Lasters I. (2007). Immunogenicity screening in protein drug development. Expert Opin. Biol. Ther. 7, 405-418.
Vargas-Madrazo E, Paz-Garcia E. (2003). An improved model of association for VH-VL immunoglobulin domains: asymmetries between VH and VL in the packing of some interface residues. J. Mol. Recognit. 16, 113-120.
Verreck F A, van de Poel A, Drijfhout J W, Amons R, Coligan J E, Konig F. (1996). Natural peptides isolated from Gly86/Va186-containing variants of HLA-DR1, -DR11, -DR13, and -DR52. Immunogenetics 43, 392-397.

### Extra References

Madeira, F., Y. M. Park, J. Lee, N. Buso, T. Gur, N. Madhusoodanan, P. Basutkar, et al. 2019. “The EMBL-EBI Search and Sequence Analysis Tools APIs in 2019.” Nucleic Acids Research, April. https://doi.org/10.1093/nar/gkz268.
Needleman, S. B., and C. D. Wunsch. 1970. “A General Method Applicable to the Search for Similarities in the Amino Acid Sequence of Two Proteins.” Journal of Molecular Biology 48 (3): 443-53.

1. An isolated anti-Ryk antibody or antibody derivative that:
a) specifically binds to a Wnt-binding domain on Ryk or specifically binds to an epitope within a region of the ectodomain of Ryk, e.g., amino-acids 35-211 of mouse Ryk (SEQ ID NO:24) or amino-acids 26-227 of human Ryk (SEQ ID NO:25), the antibody or antibody derivative comprising a light chain variable region comprising the CDR sequence set forth in SEQ ID NO:1 [RANRLVE];
b) specifically binds to the same epitope on a Wnt-binding domain on Ryk or the same epitope within a region of the ectodomain of Ryk, amino-acids 35-211 of mouse Ryk (SEQ ID NO:24) or amino-acids 26-227 of human Ryk (SEQ ID NO:25), as does a reference antibody or antibody derivative, or cross-competes for specific binding to the Wnt-binding domain on Ryk or the same epitope within a region of the ectodomain of Ryk, with a reference antibody or antibody derivative, the reference antibody or antibody derivative comprising a light chain variable region comprising the CDR sequence set forth in SEQ ID NO:1 [RANRLVE];
c) specifically binds to a Wnt-binding domain on Ryk or specifically binds to an epitope within a region of the ectodomain of Ryk, e.g., amino-acids 35-211 of mouse Ryk (SEQ ID NO:24) or amino-acids 26-227 of human Ryk (SEQ ID NO:25), said antibody or antibody derivative comprising a heavy chain variable region comprising the CDR sequence set forth in SEQ ID NO:2 [STGGGGTY], SEQ ID NO:3 [HGDSGDY] or SEQ ID NO:4 [HGDQGDY]; or
d) specifically binds to the same epitope on a Wnt-binding domain on Ryk or the same epitope within a region of the ectodomain of Ryk, e.g., amino-acids 35-211 of mouse Ryk (SEQ ID NO:24) or amino-acids 26-227 of human Ryk (SEQ ID NO:25), as does a reference antibody or antibody derivative, or cross-competes for specific binding to the Wnt-binding domain on Ryk or the same epitope within a region of the ectodomain of Ryk with a reference antibody or antibody derivative, the reference antibody or antibody derivative comprising a heavy chain variable region comprising the CDR sequence set forth in SEQ ID NO:2 [STGGGGTY], SEQ ID NO:3 [HGDSGDY] or SEQ ID NO:4 [HGDQGDY],
provided that the antibody or antibody derivative is not an isolated anti-Ryk antibody or antibody derivative disclosed and/or claimed in WO 2017/172733 A1.

2. The isolated anti-Ryk antibody or antibody derivative of claim 1, which comprises a light chain variable region comprising the CDR sequence set forth in SEQ ID NO:1.

3. (canceled)

4. The isolated anti-Ryk antibody or antibody derivative of claim 1, which comprises a heavy chain variable region comprising the CDR sequence set forth in SEQ ID NO:2 [STGGGGTY], SEQ ID NO:3 [HGDSGDY] or SEQ ID NO:4 [HGDQGDY].

5-6. (canceled)

7. The isolated anti-Ryk antibody or antibody derivative of claim 1, wherein the light chain variable region comprises an amino acid sequence comprising at least about 85% sequence identity to SEQ ID NO:11 [VL1], SEQ ID NO:12 [VL2], or SEQ ID NO:13 [VL3].

8. (canceled)

9. The isolated anti-Ryk antibody or antibody derivative of claim 1, wherein the heavy chain variable region comprises an amino acid sequence comprising at least about 85% sequence identity to SEQ ID NO:14 [VH1], SEQ ID NO:15 [VH2], SEQ ID NO:16 [VH3], SEQ ID NO:17 [VH4], or SEQ ID NO:18 [VH5].

10-11. (canceled)

12. An isolated anti-Ryk antibody or antibody derivative that:
a) specifically binds to a Wnt-binding domain on Ryk or specifically binds to an epitope within a region of the ectodomain of Ryk, e.g., amino-acids 35-211 of mouse Ryk (SEQ ID NO:24) or amino-acids 26-227 of human Ryk (SEQ ID NO:25), the antibody or antibody derivative comprises a light chain variable region comprising an amino acid sequence comprising at least about 85% sequence identity to SEQ ID NO:11 [VL1], SEQ ID NO:12 [VL2], or SEQ ID NO:13 [VL3];
b) specifically binds to the same epitope on a Wnt-binding domain on Ryk or the same epitope within a region of the ectodomain of Ryk, e.g., amino-acids 35-211 of mouse Ryk (SEQ ID NO:24) or amino-acids 26-227 of human Ryk (SEQ ID NO:25), as does a reference antibody or antibody derivative, or cross-competes for specific binding to the Wnt-binding domain on Ryk or the same epitope within a region of the ectodomain of Ryk with a reference antibody or antibody derivative, the reference antibody or antibody derivative comprising a light chain variable region comprising an amino acid sequence comprising at least about 85% sequence identity to SEQ ID NO:11 [VL1], SEQ ID NO:12 [VL2], or SEQ ID NO:13 [VL3];
c) specifically binds to a Wnt-binding domain on Ryk or specifically binds to an epitope within a region of the ectodomain of Ryk, e.g., amino-acids 35-211 of mouse Ryk (SEQ ID NO:24) or amino-acids 26-227 of human Ryk (SEQ ID NO:25), the antibody or antibody derivative comprises a heavy chain variable region that comprises an amino acid sequence comprising at least about 85% sequence identity to SEQ ID NO:14 [VH1], SEQ ID NO:15 [VH2], SEQ ID NO:16 [VH3], SEQ ID NO:17 [VH4], or SEQ ID NO:18 [VH5]; or
d) specifically binds to the same epitope on a Wnt-binding domain on Ryk or the same epitope within a region of the ectodomain of Ryk, e.g., amino-acids 35-211 of mouse Ryk (SEQ ID NO:24) or amino-acids 26-227 of human Ryk (SEQ ID NO:25), as does a reference antibody or antibody derivative, or cross-competes for specific binding to the Wnt-binding domain on Ryk or the same epitope within a region of the ectodomain of Ryk with a reference antibody or antibody derivative, the reference antibody or antibody derivative comprising a heavy chain variable region that comprises an amino acid sequence comprising at least about 85% sequence identity to SEQ ID NO:14 [VH1], SEQ ID NO:15 [VH2], SEQ ID NO:16 [VH3], SEQ ID NO:17 [VH4], or SEQ ID NO:18 [VH5],
provided that the antibody or antibody derivative is not an isolated anti-Ryk antibody or antibody derivative disclosed and/or claimed in WO 2017/172733 A1.

13-14. (canceled)

15. The isolated anti-Ryk antibody or antibody derivative of claim 12, wherein the light chain variable region comprises the amino acid sequence set forth in SEQ ID NO:11 [VL1], SEQ ID NO:12 [VL2], or SEQ ID NO:13 [VL3], and the heavy chain variable region comprises the amino acid sequence set forth in SEQ ID NO:14 [VH1], SEQ ID NO:15 [VH2], SEQ ID NO:16 [VH3], SEQ ID NO:17 [VH4], or SEQ ID NO:18 [VH5].

16-30. (canceled)

31. The isolated anti-Ryk antibody or antibody derivative of claim 1, wherein the antibody or antibody derivative is a humanized antibody, e.g., a humanized monoclonal antibody.

32-33. (canceled)

34. The isolated anti-Ryk antibody or antibody derivative of claim 1, wherein the antibody or antibody derivative inhibits or reduces Ryk binding to Wnt or the antibody or antibody derivative specifically binds to an epitope within amino acid residues 90-183 of Ryk.

35. (canceled)

36. The isolated anti-Ryk antibody or antibody derivative of claim 1, wherein the antibody or antibody derivative specifically binds to an epitope within or comprising the amino acid sequence set forth in SEQ ID NO:19 [SRTIYDPV] or an epitope within or comprising an amino acid sequence having at least about 80% sequence identity to the amino acid sequence set forth in SEQ ID NO:19 [SRTIYDPV].

37-38. (canceled)

39. The isolated anti-Ryk antibody or antibody derivative of claim 1, which has lower immunogenicity than Ab5.5 disclosed and/or claimed in WO 2017/172733 A1 in a human.

40-41. (canceled)

42. The isolated anti-Ryk antibody or antibody derivative of claim 1, which has a KD value for binding to a Ryk polypeptide ranging from about 0.01 pM to about 500 pM.

43-46. (canceled)

47. A nucleic acid sequence encoding the isolated antibody or antibody derivative of claim 1.

48-52. (canceled)

53. A method of interfering with interaction of Wnt and Ryk comprising contacting a sample comprising Wnt and Ryk with the isolated antibody or antibody derivative of claim 1, thereby interfering with the interaction of Wnt and Ryk.

54. A method for inhibiting degeneration of a neuron, the method comprising contacting the neuron with the isolated antibody or antibody derivative of claim 1, thereby inhibiting degeneration of the neuron.

55-65. (canceled)

66. A method of preventing or treating a neurological disease, disorder or injury in a subject having or being at risk of developing the neurological disease, disorder or injury comprising administering to the subject an effective amount of the isolated antibody or antibody derivative of claim 1, thereby treating the neurological disease, disorder or injury in the subject.

67. The method of claim 66, wherein the neurological disease or disorder is a neurodegenerative disease or disorder, e.g., amyotrophic lateral sclerosis, Alzheimer's disease or Parkinson's disease or the neurological injury is a spinal cord injury, a traumatic brain injury, or a peripheral nerve injury.

68. (canceled)

69. A method for modulating the directional growth of a neuron comprising contacting the neuron with the isolated antibody or antibody derivative of claim 1, thereby modulating the directional growth of the neuron.

70-73. (canceled)

74. A method of preventing or treating a cancer or tumor in a subject having or being at risk of developing the cancer or tumor comprising administering to the subject an effective amount of the isolated antibody or antibody derivative of claim 1, thereby treating or treating the cancer or tumor in the subject.

75-84. (canceled)

85. A method for assessing a Ryk polypeptide in a sample, which method comprises:
a) contacting a sample containing or suspected of containing a Ryk polypeptide with the isolated antibody or antibody derivative of claim 1; and
b) assessing binding between the Ryk polypeptide, if present in the sample, and the isolated antibody or antibody derivative, the immunoconjugate or the bispecific molecule to assess the presence, absence, level or amount of the Ryk polypeptide in the sample.

86-108. (canceled)

109. The method of claim 66, wherein the neurological disease, disorder or injury is neuropathic pain.

110-112. (canceled)
